SEQUENCE LISTING (140 sequences)

SEQ ID NO: 1 - DNA, 50 bp, synthetic construct
  source 1..50: mol_type = other DNA
gtagtaggccctcagtcatcccatttgggaacagtgcatggcggtccttc

SEQ ID NO: 2 - DNA, 50 bp, synthetic construct
  source 1..50: mol_type = other DNA
cctacaaagacaccctccgacaacaatggagagtccacatgtcccgatac

SEQ ID NO: 3 - DNA, 50 bp, synthetic construct
  source 1..50: mol_type = other DNA
tcctaatgggactaagtcatcctccaccctcacctaccttctggctgctg

SEQ ID NO: 4 - DNA, 50 bp, synthetic construct
  source 1..50: mol_type = other DNA
ccttgatctgggaattctcatcgtcgacaccatgaacgaaacaaatttct

SEQ ID NO: 5 - DNA, 50 bp, synthetic construct
  source 1..50: mol_type = other DNA
cctctttccaaataccagcttcttctttagatgtcatcacctccacaaca

SEQ ID NO: 6 - DNA, 50 bp, synthetic construct
  source 1..50: mol_type = other DNA
ccagtttaaacggccatttacttccagttgagcaggcccctgactgacac

SEQ ID NO: 7 - DNA, 50 bp, synthetic construct
  source 1..50: mol_type = other DNA
cacttctgcttctgcagcaggtgcgtgtggaggcaagctagtcctgagac

SEQ ID NO: 8 - DNA, 50 bp, synthetic construct
  source 1..50: mol_type = other DNA
agtggaggcgggacctgaacctggagctccaacttgcagatggagttctt

SEQ ID NO: 9 - DNA, 50 bp, synthetic construct
  source 1..50: mol_type = other DNA
acaggtcaatactttaaagcttagaaaaataatatcaggaatctctgctt

SEQ ID NO: 10 - DNA, 50 bp, synthetic construct
  source 1..50: mol_type = other DNA
tttttacccattttcttataagagatccctaaaagggactgaatgtatca

SEQ ID NO: 11 - DNA, 50 bp, synthetic construct
  source 1..50: mol_type = other DNA
cttgtagggttgagacaatctcagtcagctttttttaacctgtgaatgtc

SEQ ID NO: 12 - DNA, 50 bp, synthetic construct
  source 1..50: mol_type = other DNA
gtatttattgagaacatctactataagaaaaagcaattattcttcaaaag

SEQ ID NO: 13 - DNA, 50 bp, synthetic construct
  source 1..50: mol_type = other DNA
aaacaaagtggaatataacaacctcaggacacaccccagggtcacagtct

SEQ ID NO: 14 - DNA, 50 bp, synthetic construct
  source 1..50: mol_type = other DNA
aggtgtctcaccaacctcacgtccgctctgtctgcagcgtccggggtgcc

SEQ ID NO: 15 - DNA, 50 bp, synthetic construct
  source 1..50: mol_type = other DNA
gggactcattatttaaaaacaatggcaaaaacaaacaacaacaacaacaa

SEQ ID NO: 16 - DNA, 50 bp, synthetic construct
  source 1..50: mol_type = other DNA
tcaacaaatgtcagtttttacagttgtttcatggtcttttttttttacca

SEQ ID NO: 17 - DNA, 50 bp, synthetic construct
  source 1..50: mol_type = other DNA
ggtgtgtggccatttgactcttgctttctttatgtaatatcataatgacc

SEQ ID NO: 18 - DNA, 50 bp, synthetic construct
  source 1..50: mol_type = other DNA
attgtttgagactgaaactgatatatgaataggaaaatgatgtttagttt

SEQ ID NO: 19 - DNA, 50 bp, synthetic construct
  source 1..50: mol_type = other DNA
tttaaggaagattccttcctgctctagcttatcttttttagctttttttt

SEQ ID NO: 20 - DNA, 50 bp, synthetic construct
  source 1..50: mol_type = other DNA
aaaaaccaagaaacattttgttcatgtttcttttcttgtagcctcttcgg

SEQ ID NO: 21 - DNA, 50 bp, synthetic construct
  source 1..50: mol_type = other DNA
ttttagctgtgagacattagactagttattaactcttttaagcttgagtt

SEQ ID NO: 22 - DNA, 50 bp, synthetic construct
  source 1..50: mol_type = other DNA
catcaccatgcccagataatttttctatcttttttgtagagacagggttt

SEQ ID NO: 23 - DNA, 50 bp, synthetic construct
  source 1..50: mol_type = other DNA
tcccctgaggaggccaagccttttcaaatgccgttccctctgctggaata

SEQ ID NO: 24 - DNA, 50 bp, synthetic construct
  source 1..50: mol_type = other DNA
tcagccagaggtggtgccttttcctttcccaggatttccctggtcataca

SEQ ID NO: 25 - DNA, 50 bp, synthetic construct
  source 1..50: mol_type = other DNA
gggaaagagacaatttgacttcagatgccttgccagatcttcctcctcta

SEQ ID NO: 26 - DNA, 50 bp, synthetic construct
  source 1..50: mol_type = other DNA
caggccgaccgcgtccagcaggagggcgaggatgaggaagagcgcacagc

SEQ ID NO: 27 - DNA, 50 bp, synthetic construct
  source 1..50: mol_type = other DNA
gattacaggcacccaccaccacgcccagctaattttttgtatttttagta

SEQ ID NO: 28 - DNA, 50 bp, synthetic construct
  source 1..50: mol_type = other DNA
ctgttccatttttataaaattatattccctttaaaagaaataatgtttat

SEQ ID NO: 29 - DNA, 50 bp, synthetic construct
  source 1..50: mol_type = other DNA
atccatctccctgactgactaaaaccacgggtttatatagaagggaaaaa

SEQ ID NO: 30 - DNA, 50 bp, synthetic construct
  source 1..50: mol_type = other DNA
gttatccagcaaaaggcagggcaatggcattctgtttccctcatcagcct

SEQ ID NO: 31 - DNA, 50 bp, synthetic construct
  source 1..50: mol_type = other DNA
catcatgtttctgtttgcagactgggcatgtccttgcttcaaaagtaaaa

SEQ ID NO: 32 - DNA, 50 bp, synthetic construct
  source 1..50: mol_type = other DNA
tggtctcaaacctctgacctcaggtgatctgcccgcctcagccccccaaa

SEQ ID NO: 33 - DNA, 50 bp, synthetic construct
  source 1..50: mol_type = other DNA
tctgtgtatcagaccatacacagaccatatttacaagactaacttcatgt

SEQ ID NO: 34 - DNA, 50 bp, synthetic construct
  source 1..50: mol_type = other DNA
ggagggagagcgcgtttcatcatcggcggcggccacttataaaaacttct

SEQ ID NO: 35 - DNA, 50 bp, synthetic construct
  source 1..50: mol_type = other DNA
agatcataaaatgggcatctaatttccctcttgtgtactgcagtctgttc

SEQ ID NO: 36 - DNA, 50 bp, synthetic construct
  source 1..50: mol_type = other DNA
ggtgttctttgagaccttcaacgtgccggccctgttcatctccatgcagg

SEQ ID NO: 37 - DNA, 50 bp, synthetic construct
  source 1..50: mol_type = other DNA
taattctatgtttaactacttgaaaaactgccagactgttttccaaacca

SEQ ID NO: 38 - DNA, 50 bp, synthetic construct
  source 1..50: mol_type = other DNA
gacagaaacatccgcagagtgaccagggcaggtattcttgatcagatcat

SEQ ID NO: 39 - DNA, 50 bp, synthetic construct
  source 1..50: mol_type = other DNA
gacagggttgggagcaggggatgggtgctgttaacctttgagatgtccgt

SEQ ID NO: 40 - DNA, 50 bp, synthetic construct
  source 1..50: mol_type = other DNA
gtattgtacctaactgggatagctcctactcagctccaaccaactgttgc

SEQ ID NO: 41 - DNA, 50 bp, synthetic construct
  source 1..50: mol_type = other DNA
gggttcaagcgatcttcccgcctcagccttccaagtagctgggattacag

SEQ ID NO: 42 - DNA, 50 bp, synthetic construct
  source 1..50: mol_type = other DNA
cctgtcatcttgttctcagtggagctcatgattcataaacacctaagaac

SEQ ID NO: 43 - DNA, 50 bp, synthetic construct
  source 1..50: mol_type = other DNA
gcctgtctaactcagactcctttatcgtaatggatgttgtagttctatga

SEQ ID NO: 44 - DNA, 50 bp, synthetic construct
  source 1..50: mol_type = other DNA
gttatgactttaagaagttgtggaaatagaagactaaaagaaattagaca

SEQ ID NO: 45 - DNA, 50 bp, synthetic construct
  source 1..50: mol_type = other DNA
ctgactccaattagattttatagagatttataaagcagacatgtattttt

SEQ ID NO: 46 - DNA, 50 bp, synthetic construct
  source 1..50: mol_type = other DNA
tgctgggtgtgctcactgctactggggtgtcattgtttttaactcctctc

SEQ ID NO: 47 - DNA, 50 bp, synthetic construct
  source 1..50: mol_type = other DNA
tgttcattacgtgggtaatgggttcattagaagcccaaacctcagcatga

SEQ ID NO: 48 - DNA, 50 bp, synthetic construct
  source 1..50: mol_type = other DNA
acagaaacagccgtattggttacatttcagtgtttgccttatctgaacat

SEQ ID NO: 49 - DNA, 50 bp, synthetic construct
  source 1..50: mol_type = other DNA
cacagagatggcccaggaggacccagggcggacccctcggcttgggggtc

SEQ ID NO: 50 - DNA, 50 bp, synthetic construct
  source 1..50: mol_type = other DNA
atggtccgttttgcactttttttttggaattttatttagcatgtacaaat

SEQ ID NO: 51 - DNA, 50 bp, synthetic construct
  source 1..50: mol_type = other DNA
agggtccaaggttatggcattaaacatggatgaatgaggttggatttgag

SEQ ID NO: 52 - DNA, 50 bp, synthetic construct
  source 1..50: mol_type = other DNA
gatggttgttgtggttggcttcccattcaataaagacagcctctttatgc

SEQ ID NO: 53 - DNA, 50 bp, synthetic construct
  source 1..50: mol_type = other DNA
tggggcccctgctacctgaccttaaacgatgattgaaaaaacgaaaaatg

SEQ ID NO: 54 - DNA, 50 bp, synthetic construct
  source 1..50: mol_type = other DNA
ctgtatttaagggatcataaaaggaactggaaagactggtcacaatggca

SEQ ID NO: 55 - DNA, 50 bp, synthetic construct
  source 1..50: mol_type = other DNA
tgatctttgcaaaacaggtagcttgcatttcccccatcctccaccttcac

SEQ ID NO: 56 - DNA, 50 bp, synthetic construct
  source 1..50: mol_type = other DNA
tcgtttagcgtgcggagtactttttgagaagtcaagccttacatagccta

SEQ ID NO: 57 - DNA, 50 bp, synthetic construct
  source 1..50: mol_type = other DNA
atatgtgggaaagtgggtatggttttctgggcacccctcccctctcttca

SEQ ID NO: 58 - DNA, 50 bp, synthetic construct
  source 1..50: mol_type = other DNA
tgttgctaatcccaaccagcatgatttacgggaagtaaatcatctatgac

SEQ ID NO: 59 - DNA, 50 bp, synthetic construct
  source 1..50: mol_type = other DNA
aatctaggggaaaacccttttgggtgacataaggcataacctttaacagc

SEQ ID NO: 60 - DNA, 50 bp, synthetic construct
  source 1..50: mol_type = other DNA
aactctgaggaaaagtcaggaagtaaccacacacttagtctgcatagttg

SEQ ID NO: 61 - DNA, 50 bp, synthetic construct
  source 1..50: mol_type = other DNA
ttcttccatctgtttcaaaatgggtcaggaagggacaccgagcccaatag

SEQ ID NO: 62 - DNA, 50 bp, synthetic construct
  source 1..50: mol_type = other DNA
tctgggcatcgagggggctgggtgccccggtgccctgtccgagcagtgcg

SEQ ID NO: 63 - DNA, 50 bp, synthetic construct
  source 1..50: mol_type = other DNA
gccctagagacagctggacaggaagcagccagcttccagcttcttagcct

SEQ ID NO: 64 - DNA, 50 bp, synthetic construct
  source 1..50: mol_type = other DNA
catcagacctcaagaacatgaaataggtactttttattgcaggcttctgt

SEQ ID NO: 65 - DNA, 50 bp, synthetic construct
  source 1..50: mol_type = other DNA
tctgagggtgaaccacaaaatataacgcaagatccctgctttcaaggaac

SEQ ID NO: 66 - DNA, 50 bp, synthetic construct
  source 1..50: mol_type = other DNA
aaaaaaaaaaacccggccggtcacggtggctaacgcctgtaatcccagca

SEQ ID NO: 67 - DNA, 50 bp, synthetic construct
  source 1..50: mol_type = other DNA
gaaaagaaactctaaaactgggctccagtggagccagcgctaatgaatga

SEQ ID NO: 68 - DNA, 50 bp, synthetic construct
  source 1..50: mol_type = other DNA
tttggttgtatcccaaaggttttgataacttgtgtcatgattatcattct

SEQ ID NO: 69 - DNA, 50 bp, synthetic construct
  source 1..50: mol_type = other DNA
ccaatatgtacctggacctaccctactcccaccaagtcatagatgaggct

SEQ ID NO: 70 - DNA, 50 bp, synthetic construct
  source 1..50: mol_type = other DNA
tctcaaggcaactcgccgcgcagtcagcggccgactagcaggtccggatg

SEQ ID NO: 71 - DNA, 50 bp, synthetic construct
  source 1..50: mol_type = other DNA
ctgcctcagcctaccgagtagctgggactataggcgctcaccaccacact

SEQ ID NO: 72 - DNA, 50 bp, synthetic construct
  source 1..50: mol_type = other DNA
gaattcagttgggccaccctggacacgcttcttaatcttcctgaggttct

SEQ ID NO: 73 - DNA, 50 bp, synthetic construct
  source 1..50: mol_type = other DNA
ccctttaaaaaggcttagggatcactaaggggatttctagaagagcgacc

SEQ ID NO: 74 - DNA, 50 bp, synthetic construct
  source 1..50: mol_type = other DNA
tttgtgaacacacctgtgcgtatggctgtatctccacgaaaacacccagt

SEQ ID NO: 75 - DNA, 50 bp, synthetic construct
  source 1..50: mol_type = other DNA
actctgaccctcttggaagctcacatgagaaacaatatttttacaaacat

SEQ ID NO: 76 - DNA, 50 bp, synthetic construct
  source 1..50: mol_type = other DNA
tctactaaaaatacaaaaaattagctgggagtggtggcggacacctataa

SEQ ID NO: 77 - DNA, 50 bp, synthetic construct
  source 1..50: mol_type = other DNA
tgaggcttattctgaaaatccagtcctgaatgggctgacacacctaaaac

SEQ ID NO: 78 - DNA, 50 bp, synthetic construct
  source 1..50: mol_type = other DNA
agagctaaggaaagagaaagtgaaggactgagcagcaggtaaccagaatc

SEQ ID NO: 79 - DNA, 50 bp, synthetic construct
  source 1..50: mol_type = other DNA
cttaaccacacctgtcctcataaaccatttatgagtttagcagagaaagc

SEQ ID NO: 80 - DNA, 50 bp, synthetic construct
  source 1..50: mol_type = other DNA
aggtgtgctacgtaacgtgttacgaaccctaaatgcagtaggaaatcaac

SEQ ID NO: 81 - DNA, 50 bp, synthetic construct
  source 1..50: mol_type = other DNA
tattcctcctaagtgtagtacttagatctcaacattttctctactaaatt

SEQ ID NO: 82 - DNA, 50 bp, synthetic construct
  source 1..50: mol_type = other DNA
atccacctgcctcggcctcccaaagtgctgggattacagtcatgagccac

SEQ ID NO: 83 - DNA, 50 bp, synthetic construct
  source 1..50: mol_type = other DNA
agatggaaagcaggtcagaaagatcaagtttgtgtcttctccctcacacc

SEQ ID NO: 84 - DNA, 50 bp, synthetic construct
  source 1..50: mol_type = other DNA
gagcaaggagataaggaaaggcaagcttaggagaagttcagtagagtagc

SEQ ID NO: 85 - DNA, 50 bp, synthetic construct
  source 1..50: mol_type = other DNA
aattaacacagcttcaagttaggtgattctttcttctgctagttcaaatc

SEQ ID NO: 86 - DNA, 50 bp, synthetic construct
  source 1..50: mol_type = other DNA
ataacttctctgtgccttagttgtttgcatctataaagtggggttgggga

SEQ ID NO: 87 - DNA, 50 bp, synthetic construct
  source 1..50: mol_type = other DNA
agcttctggtgatgaactgtcattcgaactgggcgggattgtgaggcttc

SEQ ID NO: 88 - DNA, 50 bp, synthetic construct
  source 1..50: mol_type = other DNA
tcgtcagcactggcactggaggactcttgcagccatagggaagaggggaa

SEQ ID NO: 89 - DNA, 50 bp, synthetic construct
  source 1..50: mol_type = other DNA
ggaagagcacaggcaccaaccaactatgctggagagaagaggaaagtttg

SEQ ID NO: 90 - DNA, 50 bp, synthetic construct
  source 1..50: mol_type = other DNA
cgcaggaagccgtccgctgaacctcaggttcttgggttttcctatgcccg

SEQ ID NO: 91 - DNA, 50 bp, synthetic construct
  source 1..50: mol_type = other DNA
ctgtcttccacaatggttgaactaattcaccctcccaccaacaatgtaaa

SEQ ID NO: 92 - DNA, 50 bp, synthetic construct
  source 1..50: mol_type = other DNA
gaatccaatcactttctttatcctggtgatggagcataggtaagtaactg

SEQ ID NO: 93 - DNA, 50 bp, synthetic construct
  source 1..50: mol_type = other DNA
gggagtctgcttacacaactgatggtgtggctgtatagcatgcagcggta

SEQ ID NO: 94 - DNA, 50 bp, synthetic construct
  source 1..50: mol_type = other DNA
aacgtgcaggccgggttaagtgcctcctctctccacctacaagataattt

SEQ ID NO: 95 - DNA, 50 bp, synthetic construct
  source 1..50: mol_type = other DNA
tctttttatctttatttaaaaatacctctctgggacaactttcccctaac

SEQ ID NO: 96 - DNA, 50 bp, synthetic construct
  source 1..50: mol_type = other DNA
tcaagctcaagaaggttaagtgacttgaccagagctgtttaagtaggaaa

SEQ ID NO: 97 - DNA, 50 bp, synthetic construct
  source 1..50: mol_type = other DNA
gtccacggagaggtcacagagagctgcagcacagcaggccttttgctaat

SEQ ID NO: 98 - DNA, 50 bp, synthetic construct
  source 1..50: mol_type = other DNA
gtatataaacagagttgggagtgatgggggataggatgctgacagcacct

SEQ ID NO: 99 - DNA, 50 bp, synthetic construct
  source 1..50: mol_type = other DNA
tgttgcccaggttggagtgcagtggcgcgatcttggctcaccacaacctc

SEQ ID NO: 100 - DNA, 50 bp, synthetic construct
  source 1..50: mol_type = other DNA
atagcagtatgagaatgaactgatacagaaaattggtaccagtaacgggg

SEQ ID NO: 101 - DNA, 50 bp, synthetic construct
  source 1..50: mol_type = other DNA
ttaagttcatgggtgagttttatttccttttcgtatagatatgtagtttt

SEQ ID NO: 102 - DNA, 50 bp, synthetic construct
  source 1..50: mol_type = other DNA
gccaacatggtctctactaaaaatacaaaaattagccgtgcttggtggtg

SEQ ID NO: 103 - DNA, 50 bp, synthetic construct
  source 1..50: mol_type = other DNA
tactctgcccacttgcgctccgtcggggcacgccttgtttccttggtctc

SEQ ID NO: 104 - DNA, 50 bp, synthetic construct
  source 1..50: mol_type = other DNA
actattatagccattttacaataaagaaacggaggcttttggcagtaaac

SEQ ID NO: 105 - DNA, 50 bp, synthetic construct
  source 1..50: mol_type = other DNA
aataaataaatacaatacaggtaaagtgcttgtgtataggcagtgtggtg

SEQ ID NO: 106 - DNA, 50 bp, synthetic construct
  source 1..50: mol_type = other DNA
ctcactcctgtaatcccagcactttgggaggctgaggcaggtgggtttct

SEQ ID NO: 107 - DNA, 50 bp, synthetic construct
  source 1..50: mol_type = other DNA
gtctcacccttaccgatgtttggagtttgtaaaacccgctaatccccgac

SEQ ID NO: 108 - DNA, 50 bp, synthetic construct
  source 1..50: mol_type = other DNA
ggaggtgtgcgggggctgagtctggccaggccagcggggggagtgggagg

SEQ ID NO: 109 - DNA, 50 bp, synthetic construct
  source 1..50: mol_type = other DNA
gtcattctcaaccagggcagtcccagcacttcattttatgctgtttgtaa

SEQ ID NO: 110 - DNA, 50 bp, synthetic construct
  source 1..50: mol_type = other DNA
gctaacggaaatacatcaacaactacagcttgtgaaacaccaacagtctg

SEQ ID NO: 111 - DNA, 50 bp, synthetic construct
  source 1..50: mol_type = other DNA
aggtttagagaattctttaagaatgtctttgtgtggcatatgctttggtc

SEQ ID NO: 112 - DNA, 50 bp, synthetic construct
  source 1..50: mol_type = other DNA
aagagtatcagctcctgtttatatttctggtagaattcagctgtaaaacc

SEQ ID NO: 113 - DNA, 50 bp, synthetic construct
  source 1..50: mol_type = other DNA
ggaacaacatataatggggcctgtcggggggtcgggtggggagaggaaga

SEQ ID NO: 114 - DNA, 50 bp, synthetic construct
  source 1..50: mol_type = other DNA
tgtagccagagttaataccctcatcgtcctttgagctcagcagatgaaag

SEQ ID NO: 115 - DNA, 50 bp, synthetic construct
  source 1..50: mol_type = other DNA
ctcctttctgttgcctgcagcaaaaaagctcctttgagcaaactgatagg

SEQ ID NO: 116 - DNA, 50 bp, synthetic construct
  source 1..50: mol_type = other DNA
gcagctatttgtgtacacggcttatctccctgccagactgagaggcaacg

SEQ ID NO: 117 - DNA, 50 bp, synthetic construct
  source 1..50: mol_type = other DNA
agctgcaagtgaatccagctaacagaaatcaatattgtcttatttgaata

SEQ ID NO: 118 - DNA, 50 bp, synthetic construct
  source 1..50: mol_type = other DNA
ggaacccgaagagccttgaatgctgggccgaggaacttcactcttcatac

SEQ ID NO: 119 - DNA, 50 bp, synthetic construct
  source 1..50: mol_type = other DNA
ggtgagacacagttctgtgtttatagcttctgcgtgatatagtttcctcc

SEQ ID NO: 120 - DNA, 50 bp, synthetic construct
  source 1..50: mol_type = other DNA
gggaatatcacagcctgggctgtttggttatcttcattattaatacacac

SEQ ID NO: 121 - DNA, 50 bp, synthetic construct
  source 1..50: mol_type = other DNA
tgcaagagaggggcaccacaaatctggctgacagctctgagcaggtagat

SEQ ID NO: 122 - DNA, 50 bp, synthetic construct
  source 1..50: mol_type = other DNA
atctggtcacaggatgatgagggtgttccctctgaaaactccaaggttgc

SEQ ID NO: 123 - DNA, 50 bp, synthetic construct
  source 1..50: mol_type = other DNA
tacactcattattacaccaagcacccatgaggtctcaagtgcagtctgtt

SEQ ID NO: 124 - DNA, 50 bp, synthetic construct
  source 1..50: mol_type = other DNA
ctccagagaagaaaaagtgttgatggatgaaggagcagtacttaccctgg

SEQ ID NO: 125 - DNA, 50 bp, synthetic construct
  source 1..50: mol_type = other DNA
ttctgtttaagacatctttgcctactcccaggacaagaagctattaccta

SEQ ID NO: 126 - DNA, 50 bp, synthetic construct
  source 1..50: mol_type = other DNA
gccaggaaccggtgggattcctgggaatggggccagggagtggacagaag

SEQ ID NO: 127 - DNA, 50 bp, synthetic construct
  source 1..50: mol_type = other DNA
cttcccctggagttggacgcctcagtgactggactcttctctgaccaccc

SEQ ID NO: 128 - DNA, 50 bp, synthetic construct
  source 1..50: mol_type = other DNA
tccttggtcccattcctgacttacttaaaatcagaatttctagggatggg

SEQ ID NO: 129 - DNA, 50 bp, synthetic construct
  source 1..50: mol_type = other DNA
tgcatgagatgggagaggcaggttggtccctgggcagctctgcccgctgg

SEQ ID NO: 130 - DNA, 50 bp, synthetic construct
  source 1..50: mol_type = other DNA
tcaaaaggtaatttcgatgttatgcatttattttattttattagtaccct

SEQ ID NO: 131 - DNA, 50 bp, synthetic construct
  source 1..50: mol_type = other DNA
gaagcatgctgaattcactgtaattacaaaggtaataaaaatgagccacg

SEQ ID NO: 132 - DNA, 50 bp, synthetic construct
  source 1..50: mol_type = other DNA
aatccttttacataactttactcatctctacctctcccttccctccaaag

SEQ ID NO: 133 - DNA, 50 bp, synthetic construct
  source 1..50: mol_type = other DNA
tgctgcagagaggagacagaactctaaaaaggatgtgtgacacacagagc

SEQ ID NO: 134 - DNA, 50 bp, synthetic construct
  source 1..50: mol_type = other DNA
ctcctgatctcaagtgatttgccagcctcggcctcccaaagtgctaggat

SEQ ID NO: 135 - DNA, 50 bp, synthetic construct
  source 1..50: mol_type = other DNA
gaatgttaccttatggtatatggtaaaagggacttggcagatggaattaa

SEQ ID NO: 136 - DNA, 50 bp, synthetic construct
  source 1..50: mol_type = other DNA
ttcattttgccacaaagcaaggaggtatgtgagtcgtttcaaggagcatg

SEQ ID NO: 137 - DNA, 50 bp, synthetic construct
  source 1..50: mol_type = other DNA
cgtgagccgatatcatgccaccgcactccagcctgggtgacagagtgaga

SEQ ID NO: 138 - DNA, 50 bp, synthetic construct
  source 1..50: mol_type = other DNA
aatgtaaccaacctaatccagaacacagggcagctagggctgcctcctgg

SEQ ID NO: 139 - DNA, 50 bp, synthetic construct
  source 1..50: mol_type = other DNA
aggtgtttttataatcatactcatgcttcattgtttttagcagataaagc

SEQ ID NO: 140 - DNA, 50 bp, synthetic construct
  source 1..50: mol_type = other DNA
atgtgttcaatcttgaatcaattaccagagtccaagttgatgaagtgttc

(19) United States
(12) Patent Application Publication
(10) Pub. No.: US 2026/0209858 A1
(43) Pub. Date: Jul. 23, 2026

(54) COLORECTAL CANCER RISK ASSESSMENT

(71) Applicant: RHY GENETYPE PTY LTD, Southbank, Victoria (AU)

(72) Inventors: Aviv GAFNI, Caulfield North, Victoria (AU); Erike SPAETH, Richmond, TX (US); Gillian Sue DITE, Thornbury, Victoria (AU); Richard ALLMAN, Manor Lakes, Victoria (AU); Chi Kuen WONG, Carlton, Victoria (AU)

(73) Assignee: RHY GENETYPE PTY LTD, Southbank, Victoria (AU)

(21) Appl. No.: 19/144,115

(22) PCT Filed: Dec. 22, 2023
(86) PCT No.: PCT/AU2023/051355

§ 371 Date: Jun. 27, 2025

Related U.S. Application Data
(60) Provisional application No. 63/477,266, filed on Dec. 27, 2022.

(51) Int. Cl. C12Q 1/6886; G16B 20/20; G16B 20/40; G16H 10/20; G16H 40/67; G16H 50/20; G16H 50/30; G16H 50/70

(52) U.S. Cl. CPC C12Q 1/6886; G16B 20/20; G16B 20/40; G16H 10/20; G16H 40/67; G16H 50/20; G16H 50/30; G16H 50/70; C12Q 2600/156; C12Q 2600/172

(57) ABSTRACT

The present disclosure relates to methods for assessing the risk of a human subject for developing colorectal cancer.

### FIELD OF THE INVENTION

The present disclosure relates to methods for assessing the risk of a human subject for developing colorectal cancer.

### BACKGROUND OF THE INVENTION

Colorectal cancer has become the second most common cancer-related death globally (Keum et al., 2019). Despite the fact that colorectal cancer develops slowly over many years (which should make screening and prevention more feasible), focusing additional screening efforts on people who have a family history of colorectal cancer or are known to have one of the rare high-penetrance variants is misguided (Schreuders et al., 2015; Shaukat et al., 2022). Approximately 60-65% of colorectal cancer patients have sporadic disease, meaning that the cancer occurred in a person not known to be at increased risk (Jasperson et al., 2010).

The genetic factors that confer increased risk of colorectal cancer can be either rare high-penetrance variants or common low-penetrance variants. The rare variants account for only 5-7% of colorectal cancer cases and cause hereditary colorectal cancers, also known as Lynch syndrome and familial adenomatous polyposis (Dekker et al., 2019; Syngal et al., 2015). The common low-penetrance variants, also called single-nucleotide polymorphisms (SNPs), have been identified by genome-wide association studies.

To increase screening efficiency and to decrease colorectal cancer mortality there, is a requirement for improved methods for assessing the risk of a human subject for developing colorectal cancer.

### SUMMARY OF THE INVENTION

The present inventors have identified improved methods of assessing the risk of a human subject for developing colorectal cancer.

In one aspect, the present invention provides a method for assessing the risk of a human subject for developing colorectal cancer comprising:
i) performing a genetic risk assessment of the subject, wherein the genetic risk assessment involves detecting, in a biological sample derived from the subject, the presence of at least 50 single nucleotide polymorphisms, selected from Table 1 or a polymorphism in linkage disequilibrium with one or more thereof, associated with a risk of a human subject for developing colorectal cancer,
ii) performing a clinical risk assessment of the subject for developing colorectal cancer, and
iii) combining the genetic risk assessment and the clinical risk assessment to obtain the risk of a human subject for developing colorectal cancer.

In an embodiment, the genetic risk assessment comprises detecting the presence of at least 75, at least 100, at least 120, or each of the single nucleotide polymorphisms selected from Table 1 or a polymorphism in linkage disequilibrium with one or more thereof.

In an embodiment, the genetic risk assessment comprises detecting the presence of each of the single nucleotide polymorphisms provided in Table 1.

In an embodiment, performing the clinical risk factor assessment involves obtaining information from the subject on one or more of medical history of colorectal cancer and/or polyps, age, family history of colorectal cancer and/or polyps and/or other cancer including the age of the relative at the time of diagnosis, results of previous colonoscopy and/or sigmoidoscopy, results of previous faecal occult blood test, weight, body mass index, height, sex, alcohol consumption history, smoking history, exercise history, diet, has the subject ever smoked, time since last colorectal cancer screen, blood triglyceride levels, prevalence of inflammatory bowel disease, race/ethnicity, aspirin and other NSAID use, implementation of estrogen replacement and use of oral contraceptives.

In an embodiment, performing the clinical risk assessment involves obtaining information from the subject on first-degree relatives' history of colorectal cancer.

In an embodiment, the subject has had a positive fecal occult blood test.

In an embodiment, the subject is at least 40 years old.

In an embodiment, the subject has a family history of colorectal cancer and is at least 30 years of age.

In an embodiment, the results of the risk assessment indicate that the subject should be enrolled in a screening program or subjected to more frequent screening.

In an embodiment, the polymorphism in linkage disequilibrium has linkage disequilibrium above 0.9. In an embodiment, the polymorphism in linkage disequilibrium has linkage disequilibrium of 1.

In an embodiment, the method further comprises comparing the risk to a pre-determined threshold.

In an embodiment, the genetic risk assessment produces a polygenic risk score (PRS).

In an embodiment, the polygenic risk score is calculated as the weighted sum of the effect allele counts for the SNPs in the PRS:

$${\sum }_{j = 1}^{p} {β}_{j} {G}_{ij}$$

where βⱼ is the weight for SNP j, Gᵢⱼ is the count (0, 1, 2) of the effect alleles of SNP j for individual i, and p is the number of SNPs in the PRS, and then PRS is standardised to have a mean of 0 and standard deviation of 1 by subtracting the population mean and dividing by the population standard deviation:

$$standardised PRS = \frac{P R {S}_{raw} - P R {S}_{\overline{x}}}{P R {S}_{sd}} ,$$

where PRS_{raw} is the individual's raw PRS, PRS_{x̅} is the population mean of PRS_{raw}, and PRS_{sd} is the population standard deviation of PRS_{raw}.

In an embodiment, the subject is female and the clinical and genetic relative risk assessments are combined by determining:

$$R {R}_{fhprs \_ w} = {e}^{( PCDE 1 \times prs ) + ( PDCE 2 \times deg 1 )}$$

where:
PDCE1 is a predetermined β coefficient for the genetic risk assessment for a female subject,
PDCE2 is a predetermined β coefficient for a female subject who has at least one first-degree relative who has, or has had, colorectal cancer, and
deg1 identifies whether the subject has one or more first-degree relatives who have, or who have had, colorectal cancer.

In an embodiment, the subject is male and the clinical and genetic relative risk assessments are combined by determining:

$$R {R}_{fhprs \_ w} = {e}^{( PCDE 3 \times prs ) + ( PDCE 4 \times deg 1 )}$$

where:
PDCE3 is a predetermined β coefficient for the genetic risk assessment for a male subject,
PDCE4 is a predetermined β coefficient for a male subject who has at least one first-degree relative who has, or has had, colorectal cancer, and
deg1 identifies whether the subject has one or more first-degree relatives who have, or who have had, colorectal cancer.

In an embodiment, the polygenic risk score is determined using an odds ratio (OR) for each effect allele and effect allele frequency (p).

In an embodiment, for each polymorphism the unscaled population average risk (μ) is calculated as: μ=(1−p)²+2p(1−p)OR+p²OR².

In an embodiment, an adjusted risk for each polymorphism is calculated as

$$\frac{O {R}^{N}}{μ} ,$$

where N is the number of effect alleles.

In an embodiment, the polygenic risk score is determined by combining the adjusted risk for each polymorphism.

In an embodiment, the adjusted risk for each polymorphism are combined by multiplication to produce prs_rr.

In an embodiment, the clinical risk assessment based on whether the subject has or does not have at least one first degree relative who has, or has had, colorectal cancer (fh_rr).

In an embodiment, the genetic risk assessment and the clinical risk assessment are combined using the formula crc_rr=prs_rr×fh_rr.

In an embodiment, the subject is female and the clinical and genetic relative risk assessments are combined by determining:

$$R {R}_{multi \_ w} = {e}^{( PDCE 5 \times prs ) + ( PDCE 6 \times deg 1 ) + ( PDCE 7 \times smoke ) + ( PDCE 8 \times screen ) + ( PDCE 9 \times ( trigly - 3.296 ) )}$$

where:
PDCE5 is a predetermined β coefficient for the genetic risk assessment for a female subject,
PDCE6 is a predetermined β coefficient for a female subject who has at least one first-degree relative who has, or has had, colorectal cancer,
PDCE7 is a predetermined β coefficient for a female subject who has been, or is, a smoker,
PDCE8 is a predetermined β coefficient for a female subject who has had a colorectal cancer screen in the past 10 years,
PDCE9 is a predetermined β coefficient for a female subject's triglyceride levels (mmol/L),
deg1 is if the female subject has one or more first-degree relatives who have, or who have had, colorectal cancer
smoke is if the female subject has ever smoked,
screen is if the female subject has had a colorectal screen in the last, for example, 10 years, and
trigly is the female subject's blood triglyceride level in mmol/L.

In an embodiment, the subject is male and the clinical and genetic relative risk assessments are combined by determining:

$$R {R}_{multi \_ m} = {e}^{( PDCE 10 \times prs ) + ( PDCE 11 \times deg 1 ) + ( PDCE 12 \times smoke ) + ( PDCE 13 \times screen ) + ( PDCE 14 \times ( bmi - 3.296 ) )}$$

where:
PDCE10 is a predetermined β coefficient for the genetic risk assessment for a male subject,
PDCE11 is a predetermined β coefficient for a male subject who has at least one first-degree relative who has, or has had, colorectal cancer,
PDCE12 is a predetermined β coefficient for a male subject who has been, or is, a smoker,
PDCE13 is a predetermined β coefficient for a male subject who has had a colorectal cancer screen in the past 10 years,
PDCE14 is a predetermined β coefficient for a male subject's body mass index (natural log of kg/m²),
deg1 is if the male subject has one or more first-degree relatives who have, or who have had, colorectal cancer
smoke is if the male subject has ever smoked,
screen is if the male subject has had a colorectal screen in the last, for example, 10 years, and
bmi is the subject's body mass index expressed as the natural log of kg/m².

In an embodiment, the method comprises determining one or more or all of the absolute 5-year risk, the absolute 10-year risk, the absolute remaining lifetime risk (to age 90) or the absolute full-lifetime risk (to age 90).

In an aspect, the present invention provides a computer-implemented method for assessing the absolute risk of a human subject for developing colorectal cancer, the method operable in a computing system comprising a processor and a memory, the method comprising:
receiving clinical risk data and genetic risk data for the subject, wherein the clinical and genetic risk data was obtained by a method of the invention;
processing the data to combine the clinical risk data with the genetic risk data to obtain the relative risk of a human subject for developing colorectal cancer;
outputting the absolute risk of a human subject for developing colorectal cancer.

In an embodiment, the clinical risk data and genetic risk data for the subject is received from a user interface coupled to the computing system.

In an embodiment, the clinical risk data and genetic risk data for the subject is received from a remote device across a wireless communications network.

In an embodiment, outputting comprises outputting information to a user interface coupled to the computing system.

In an embodiment, the computer-implemented method comprises determining a genetic risk score based on genetic data derived from a biological sample taken from the subject.

Also provided is a computer-readable storage medium storing executable code, wherein when a processor executes the code, the processor is caused to perform the computer-implemented method of the invention.

In an aspect, the present invention provides a device for assessing the risk of a human subject developing colorectal cancer, the device comprising:
a processor; and
a memory device storing executable code, the memory being accessible to the processor;
   wherein, when caused to execute the executable code stored in the memory device, the processor is caused to perform a method of the invention.

In an embodiment, the device further comprises a display component, wherein the processor is further caused to display the colorectal cancer risk score of the subject for developing colorectal cancer on the display component.

In an embodiment, the device further comprises a communications module, wherein the processor is further caused to communicate the colorectal cancer risk score of the subject for developing colorectal cancer to an external device via the communications module.

In an aspect, the present invention provides a method for determining the need for routine diagnostic testing of a human subject for colorectal cancer comprising assessing the risk of the subject for developing colorectal cancer using a method of the invention.

In an aspect, the present invention provides a method of screening for colorectal cancer in a human subject, the method comprising assessing the risk of the subject for developing colorectal cancer using a method of the invention, and routinely screening for colorectal cancer in the subject if they are assessed as having a risk for developing colorectal cancer.

In an aspect, the present invention provides a method for determining the need of a human subject for prophylactic anti-colorectal cancer therapy comprising assessing the risk of the subject for developing colorectal cancer using a method of the invention.

In an aspect, the present invention provides a method for preventing colorectal cancer in a human subject, the method comprising assessing the risk of the subject for developing colorectal cancer using a method of the invention, and administering an anti-colorectal cancer therapy to the subject if they are assessed as having a risk for developing colorectal cancer.

Also provided is an anti-colorectal cancer therapy for use in preventing colorectal cancer in a human subject at risk thereof, wherein the subject is assessed as having a risk for developing colorectal cancer using a method of the invention.

In an aspect, the present invention provides a method for stratifying a group of human subjects for a clinical trial of a candidate therapy, the method comprising assessing the individual risk of the subjects for developing colorectal cancer using a method of the invention, and using the results of the assessment to select subjects more likely to be responsive to the therapy.

In a further aspect, the present invention provides a genetic array comprising at least one, at least 5, at least 10, at least 25, at least 50, at least 75, at least 100, or at least 125, probe(s) comprising, independently, a sequence of nucleotides selected from those provided as SEQ ID NOs 1 to 140. In an embodiment, the genetic array comprises at least 140 probes, wherein the at least 140 probes comprise, independently, each of the nucleotides provided as SEQ ID NOs 1 to 140. In an embodiment, the probe(s) are between 50 and 100 nucleotides in length. In an embodiment, the probe(s) are 50 nucleotides in length.

Any embodiment herein shall be taken to apply mutatis mutandis to any other embodiment unless specifically stated otherwise.

The present invention is not to be limited in scope by the specific embodiments described herein, which are intended for the purpose of exemplification only. Functionally equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The invention is hereinafter described by way of the following non-limiting Examples and with reference to the accompanying figures.

### BRIEF DESCRIPTION OF THE ACCOMPANYING DRAWINGS

FIG. 1. Standardised incidence ratios for quintiles of risk for (A) 10-year risk for the model using the 45-SNP PRS and family history, (B) 10-year risk for the model using the 140-SNP PRS and family history, (C) full-lifetime risk for the model using the 45-SNP PRS and family history, and (D) full-lifetime risk for the model using the 140-SNP PRS and family history. Data for panels (A) and (C) is from Gafni et al. (2021).

FIG. 2. Scaled Schoenfeld residuals by age in the first imputation dataset for men: (A) first-degree family history and (B) 140-SNP polygenic risk score in the new family history and PRS model; (C) first-degree family history and (D) 140-SNP PRS in the new multivariable model. Note: PRS, polygenic risk score; SNP, single-nucleotide polymorphism.

FIG. 3. Nelson-Aalen cumulative hazard function and Cox-Snell residuals from the first imputation dataset: (A) new family history and PRS model and (B) new multivariable model for women; (C) new family history and PRS model and (D) new multivariable model for men. Note: PRS, polygenic risk score.

FIG. 4. Nelson-Aalen cumulative hazard plots for women: (A) average risk, (B) family history model, (C) current family history and PRS model, (D) new family history and PRS model and (E) new multivariable model. Note: PRS, polygenic risk score.

FIG. 5. Nelson-Aalen cumulative hazard plots for women: (A) average risk, (B) family history model, (C) current family history and PRS model, (D) new family history and PRS model and (E) new multivariable model. Note: PRS, polygenic risk score.

FIG. 6. Calibration plots for women: (A) average risk, (B) family history model, (C) current family history and PRS model, (D) new family history and PRS model and (E) new multivariable model. Note: PRS, polygenic risk score.

FIG. 7. Calibration plots for men: (A) average risk, (B) family history model, (C) current family history and PRS model, (D) new family history and PRS model and (E) new multivariable model. Note: PRS, polygenic risk score.

FIG. 8. Standardised incidence ratios compared to population incidence rates for the first four quintiles of risk and the top two deciles of risk: (A) new family history and PRS model and (B) new multivariable model for women; (C) new family history and PRS model and (D) new multivariable model for men.

FIG. 9. Decision curves for the family history model, the new family history and PRS model and the new multivariable model for (A) women and (B) men. Note: PRS, polygenic risk score.

KEY TO THE SEQUENCE LISTING

| SEQ ID | | |
|---|---|---|
| NO | Name (Variant) | Sequence (Probe, DNA) |
| 1 | 10:101315166_G/A | GTAGTAGGCCCTCAGTCATCCCATTTGGGAACAGTGCATGGCG |
| | | GTCCTTC |
| 2 | 12:6406904_T/C | CCTACAAAGACACCCTCCGACAACAATGGAGAGTCCACATGTC |
| | | CCGATAC |
| 3 | 6:31010185_T/C | TCCTAATGGGACTAAGTCATCCTCCACCCTCACCTACCTTCTG |
| | | GCTGCTG |
| 4 | 6:31315512_G/A | CCTTGATCTGGGAATTCTCATCGTCGACACCATGAACGAAACA |
| | | AATTTCT |
| 5 | 10:8663875_C/T | CCTCTTTCCAAATACCAGCTTCTTCTTTAGATGTCATCACCTCC |
| | | ACAACA |
| 6 | 16:86252544_G/A | CCAGTTTAAACGGCCATTTACTTCCAGTTGAGCAGGCCCCTGA |
| | | CTGACAC |
| 7 | 10:81046265_T/C | CACTTCTGCTTCTGCAGCAGGTGCGTGTGGAGGCAAGCTAGT |
| | | CCTGAGAC |
| 8 | 15:67007018_C/G | AGTGGAGGCGGGACCTGAACCTGGAGCTCCAACTTGCAGATG |
| | | GAGTTCTT |
| 9 | 5:125988175_G/A | ACAGGTCAATACTTTAAAGCTTAGAAAAATAATATCAGGAATCT |
| | | CTGCTT |
| 10 | 6:55566108_A/G | TTTTTACCCATTTTCTTATAAGAGATCCCTAAAAGGGACTGAAT |
| | | GTATCA |
| 11 | 6:29809860_G/A | CTTGTAGGGTTGAGACAATCTCAGTCAGCTTTTTTTAACCTGTG |
| | | AATGTC |
| 12 | 13:73997961_A/G | GTATTTATTGAGAACATCTACTATAAGAAAAAGCAATTATTCTTC |
| | | AAAAG |
| 13 | 14:54369299_G/A | AAACAAAGTGGAATATAACAACCTCAGGACACACCCCAGGGTC |
| | | ACAGTCT |
| 14 | 17:80394556_G/A | AGGTGTCTCACCAACCTCACGTCCGCTCTGTCTGCAGCGTCCG |
| | | GGGTGCC |
| 15 | 13:34092164_C/T | GGGACTCATTATTTAAAAACAATGGCAAAAACAAACAACAACAA |
| | | CAACAA |
| 16 | 13:73649152_A/G | TCAACAAATGTCAGTTTTTACAGTTGTTTCATGGTCTTTTTTTTT |
| | | TACCA |
| 17 | 20:8568071_C/T | GGTGTGTGGCCATTTGACTCTTGCTTTCTTTATGTAATATCATA |
| | | ATGACC |
| 18 | 11:100717136_G/A | ATTGTTTGAGACTGAAACTGATATATGAATAGGAAAATGATGTT |
| | | TAGTTT |
| 19 | 17:814243_A/T | TTTAAGGAAGATTCCTTCCTGCTCTAGCTTATCTTTTTTAGCTTT |
| | | TTTTT |
| 20 | 15:68060389_T/G | AAAAACCAAGAAACATTTTGTTCATGTTTCTTTTCTTGTAGCCTC |
| | | TTCGG |
| 21 | 2:48686695_T/A | TTTTAGCTGTGAGACATTAGACTAGTTATTAACTCTTTTAAGCTT |
| | | GAGTT |
| 22 | 12:31594813_C/T | CATCACCATGCCCAGATAATTTTTCTATCTTTTTTGTAGAGACA |
| | | GGGTTT |
| 23 | 11:74409077_T/C | TCCCCTGAGGAGGCCAAGCCTTTTCAAATGCCGTTCCCTCTGC |
| | | TGGAATA |
| 24 | 7:46094089_C/T | TCAGCCAGAGGTGGTGCCTTTTCCTTTCCCAGGATTTCCCTGG |
| | | TCATACA |
| 25 | 3:133701119_G/A | GGGAAAGAGACAATTTGACTTCAGATGCCTTGCCAGATCTTCC |
| | | TCCTCTA |
| 26 | 17:10707241_G/A | CAGGCCGACCGCGTCCAGCAGGAGGGCGAGGATGAGGAAGA |
| | | GCGCACAGC |
| 27 | 10:52648454_C/T | GATTACAGGCACCCACCACCACGCCCAGCTAATTTTTTGTATTT |
| | | TTAGTA |
| 28 | 7:46926695_C/T | CTGTTCCATTTTTATAAAATTATATTCCCTTTAAAAGAAATAATGT |
| | | TTAT |
| 29 | 9:113671403_T/C | ATCCATCTCCCTGACTGACTAAAACCACGGGTTTATATAGAAG |
| | | GGAAAAA |
| 30 | 20:7740976_A/G | GTTATCCAGCAAAAGGCAGGGCAATGGCATTCTGTTTCCCTCA |
| | | TCAGCCT |
| 31 | 10:101351704_A/G | CATCATGTTTCTGTTTGCAGACTGGGCATGTCCTTGCTTCAAAA |
| | | GTAAAA |
| 32 | 10:114722621_G/A | TGGTCTCAAACCTCTGACCTCAGGTGATCTGCCCGCCTCAGCC |
| | | CCCCAAA |
| 33 | 10:8739580_T/A | TCTGTGTATCAGACCATACACAGACCATATTTACAAGACTAACT |
| | | TCATGT |
| 34 | 3:133748789_T/C | GGAGGGAGAGCGCGTTTCATCATCGGCGGCGGCCACTTATAA |
| | | AAACTTCT |
| 35 | 12:43134191_A/G | AGATCATAAAATGGGCATCTAATTTCCCTCTTGTGTACTGCAGT |
| | | CTGTTC |
| 36 | 2:98275354_G/A | GGTGTTCTTTGAGACCTTCAACGTGCCGGCCCTGTTCATCTCC |
| | | ATGCAGG |
| 37 | 8:117790914_C/A | TAATTCTATGTTTAACTACTTGAAAAACTGCCAGACTGTTTTCCA |
| | | AACCA |
| 38 | 4:145659064_T/C | GACAGAAACATCCGCAGAGTGACCAGGGCAGGTATTCTTGATC |
| | | AGATCAT |
| 39 | 18:46453156_A/T | GACAGGGTTGGGAGCAGGGGATGGGTGCTGTTAACCTTTGAG |
| | | ATGTCCGT |
| 40 | 2:199612407_T/C | GTATTGTACCTAACTGGGATAGCTCCTACTCAGCTCCAACCAA |
| | | CTGTTGC |
| 41 | 1:55246035_T/C | GGGTTCAAGCGATCTTCCCGCCTCAGCCTTCCAAGTAGCTGGG |
| | | ATTACAG |
| 42 | 16:86339315_T/C | CCTGTCATCTTGTTCTCAGTGGAGCTCATGATTCATAAACACCT |
| | | AAGAAC |
| 43 | 10:114288619_T/C | GCCTGTCTAACTCAGACTCCTTTATCGTAATGGATGTTGTAGTT |
| | | CTATGA |
| 44 | 12:51171090_A/G | GTTATGACTTTAAGAAGTTGTGGAAATAGAAGACTAAAAGAAAT |
| | | TAGACA |
| 45 | 5:40280076_G/A | CTGACTCCAATTAGATTTTATAGAGATTTATAAAGCAGACATGT |
| | | ATTTTT |
| 46 | 3:112916918_C/T | TGCTGGGTGTGCTCACTGCTACTGGGGTGTCATTGTTTTTAAC |
| | | TCCTCTC |
| 47 | 7:45136423_T/C | TGTTCATTACGTGGGTAATGGGTTCATTAGAAGCCCAAACCTC |
| | | AGCATGA |
| 48 | 15:32992836_G/A | ACAGAAACAGCCGTATTGGTTACATTTCAGTGTTTGCCTTATCT |
| | | GAACAT |
| 49 | 19:49218602_C/T | CACAGAGATGGCCCAGGAGGACCCAGGGCGGACCCCTCGGC |
| | | TTGGGGGTC |
| 50 | 3:112903888_A/G | ATGGTCCGTTTTGCACTTTTTTTTTGGAATTTTATTTAGCATGTA |
| | | CAAAT |
| 51 | 4:94938618_C/A | AGGGTCCAAGGTTATGGCATTAAACATGGATGAATGAGGTTGG |
| | | ATTTGAG |
| 52 | 13:78609615_T/C | GATGGTTGTTGTGGTTGGCTTCCCATTCAATAAAGACAGCCTC |
| | | TTTATGC |
| 53 | 20:57475191_A/G | TGGGGCCCCTGCTACCTGACCTTAAACGATGATTGAAAAAACG |
| | | AAAAATG |
| 54 | 4:106128760_G/A | CTGTATTTAAGGGATCATAAAAGGAACTGGAAAGACTGGTCAC |
| | | AATGGCA |
| 55 | 12:115100714_T/C | TGATCTTTGCAAAACAGGTAGCTTGCATTTCCCCCATCCTCCAC |
| | | CTTCAC |
| 56 | 5:98206082_T/A | TCGTTTAGCGTGCGGAGTACTTTTTGAGAAGTCAAGCCTTACAT |
| | | AGCCTA |
| 57 | 9:22103183_G/T | ATATGTGGGAAAGTGGGTATGGTTTTCTGGGCACCCCTCCCCT |
| | | CTCTTCA |
| 58 | 6:35569562_A/G | TGTTGCTAATCCCAACCAGCATGATTTACGGGAAGTAAATCATC |
| | | TATGAC |
| 59 | 8:117630683_A/C | AATCTAGGGGAAAACCCTTTTGGGTGACATAAGGCATAACCTT |
| | | TAACAGC |
| 60 | 1:222112634_A/G | AACTCTGAGGAAAAGTCAGGAAGTAACCACACACTTAGTCTGC |
| | | ATAGTTG |
| 61 | 14:59189361_G/A | TTCTTCCATCTGTTTCAAAATGGGTCAGGAAGGGACACCGAGC |
| | | CCAATAG |
| 62 | 20:60932414_T/C | TCTGGGCATCGAGGGGGCTGGGTGCCCCGGTGCCCTGTCCG |
| | | AGCAGTGCG |
| 63 | 11:61549025_G/A | GCCCTAGAGACAGCTGGACAGGAAGCAGCCAGCTTCCAGCTT |
| | | CTTAGCCT |
| 64 | 15:33156386_G/A | CATCAGACCTCAAGAACATGAAATAGGTACTTTTTATTGCAGGC |
| | | TTCTGT |
| 65 | 20:6376457_G/C | TCTGAGGGTGAACCACAAAATATAACGCAAGATCCCTGCTTTC |
| | | AAGGAAC |
| 66 | 19:41871573_G/A | AAAAAAAAAAACCCGGCCGGTCACGGTGGCTAACGCCTGTAAT |
| | | CCCAGCA |
| 67 | 6:12292772_G/T | GAAAAGAAACTCTAAAACTGGGCTCCAGTGGAGCCAGCGCTAA |
| | | TGAATGA |
| 68 | 11:101656397_T/A | TTTGGTTGTATCCCAAAGGTTTTGATAACTTGTGTCATGATTAT |
| | | CATTCT |
| 69 | 12:6421174_A/T | CCAATATGTACCTGGACCTACCCTACTCCCACCAAGTCATAGAT |
| | | GAGGCT |
| 70 | 15:33010736_G/A | TCTCAAGGCAACTCGCCGCGCAGTCAGCGGCCGACTAGCAGG |
| | | TCCGGATG |
| 71 | 6:31449620_C/T | CTGCCTCAGCCTACCGAGTAGCTGGGACTATAGGCGCTCACC |
| | | ACCACACT |
| 72 | 12:12035649_C/T | GAATTCAGTTGGGCCACCCTGGACACGCTTCTTAATCTTCCTG |
| | | AGGTTCT |
| 73 | 5:1296486_A/G | CCCTTTAAAAAGGCTTAGGGATCACTAAGGGGATTTCTAGAAG |
| | | AGCGACC |
| 74 | 20:62308612_T/G | TTTGTGAACACACCTGTGCGTATGGCTGTATCTCCACGAAAAC |
| | | ACCCAGT |
| 75 | 20:6762221_C/T | ACTCTGACCCTCTTGGAAGCTCACATGAGAAACAATATTTTTAC |
| | | AAACAT |
| 76 | 19:33519927_T/G | TCTACTAAAAATACAAAAAATTAGCTGGGAGTGGTGGCGGACA |
| | | CCTATAA |
| 77 | 11:111156836_T/C | TGAGGCTTATTCTGAAAATCCAGTCCTGAATGGGCTGACACAC |
| | | CTAAAAC |
| 78 | 6:30758466_A/G | AGAGCTAAGGAAAGAGAAAGTGAAGGACTGAGCAGCAGGTAA |
| | | CCAGAATC |
| 79 | 12:4388271_C/T | CTTAACCACACCTGTCCTCATAAACCATTTATGAGTTTAGCAGA |
| | | GAAAGC |
| 80 | 12:4400808_C/T | AGGTGTGCTACGTAACGTGTTACGAACCCTAAATGCAGTAGGA |
| | | AATCAAC |
| 81 | 9:101679752_T/G | TATTCCTCCTAAGTGTAGTACTTAGATCTCAACATTTTCTCTACT |
| | | AAATT |
| 82 | 19:16417198_C/T | ATCCACCTGCCTCGGCCTCCCAAAGTGCTGGGATTACAGTCAT |
| | | GAGCCAC |
| 83 | 14:54419106_A/C | AGATGGAAAGCAGGTCAGAAAGATCAAGTTTGTGTCTTCTCCC |
| | | TCACACC |
| 84 | 3:40915239_A/G | GAGCAAGGAGATAAGGAAAGGCAAGCTTAGGAGAAGTTCAGT |
| | | AGAGTAGC |
| 85 | 12:4368607_T/C | AATTAACACAGCTTCAAGTTAGGTGATTCTTTCTTCTGCTAGTT |
| | | CAAATC |
| 86 | 2:219191256_T/C | ATAACTTCTCTGTGCCTTAGTTGTTTGCATCTATAAAGTGGGGT |
| | | TGGGGA |
| 87 | 7:47511161_A/G | AGCTTCTGGTGATGAACTGTCATTCGAACTGGGGGGGATTGTG |
| | | AGGCTTC |
| 88 | 6:32191339_C/T | TCGTCAGCACTGGCACTGGAGGACTCTTGCAGCCATAGGGAA |
| | | GAGGGGAA |
| 89 | 8:128571855_G/T | GGAAGAGCACAGGCACCAACCAACTATGCTGGAGAGAAGAGG |
| | | AAAGTTTG |
| 90 | 1:38455891_G/C | CGCAGGAAGCCGTCCGCTGAACCTCAGGTTCTTGGGTTTTCCT |
| | | ATGCCCG |
| 91 | 11:10286755_A/C | CTGTCTTCCACAATGGTTGAACTAATTCACCCTCCCACCAACAA |
| | | TGTAAA |
| 92 | 2:159964552_T/C | GAATCCAATCACTTTCTTTATCCTGGTGATGGAGCATAGGTAAG |
| | | TAACTG |
| 93 | 12:57533690_C/A | GGGAGTCTGCTTACACAACTGATGGTGTGGCTGTATAGCATGC |
| | | AGCGGTA |
| 94 | 20:6699595_T/G | AACGTGCAGGCCGGGTTAAGTGCCTCCTCTCTCCACCTACAAG |
| | | ATAATTT |
| 95 | 14:54445157_G/A | TCTTTTTATCTTTATTTAAAAATACCTCTCTGGGACAACTTTCCC |
| | | CTAAC |
| 96 | 17:809643_G/A | TCAAGCTCAAGAAGGTTAAGTGACTTGACCAGAGCTGTTTAAG |
| | | TAGGAAA |
| 97 | 5:134467220_C/T | GTCCACGGAGAGGTCACAGAGAGCTGCAGCACAGCAGGCCTT |
| | | TTGCTAAT |
| 98 | 15:67402824_T/C | GTATATAAACAGAGTTGGGAGTGATGGGGGATAGGATGCTGAC |
| | | AGCACCT |
| 99 | 12:111973358_A/G | TGTTGCCCAGGTTGGAGTGCAGTGGCGCGATCTTGGCTCACC |
| | | ACAACCTC |
| 100 | 20:42666475_C/T | ATAGCAGTATGAGAATGAACTGATACAGAAAATTGGTACCAGTA |
| | | ACGGGG |
| 101 | 20:33213196_A/C | TTAAGTTCATGGGTGAGTTTTATTTCCTTTTCGTATAGATATGTA |
| | | GTTTT |
| 102 | 20:49055318_C/T | GCCAACATGGTCTCTACTAAAAATACAAAAATTAGCCGTGCTTG |
| | | GTGGTG |
| 103 | 20:47340117_A/G | TACTCTGCCCACTTGCGCTCCGTCGGGGCACGCCTTGTTTCCT |
| | | TGGTCTC |
| 104 | 20:48983697_C/T | ACTATTATAGCCATTTTACAATAAAGAAACGGAGGCTTTTGGCA |
| | | GTAAAC |
| 105 | 20:49256285_C/T | AATAAATAAATACAATACAGGTAAAGTGCTTGTGTATAGGCAGT |
| | | GTGGTG |
| 106 | 11:74427921_C/T | CTCACTCCTGTAATCCCAGCACTTTGGGAGGCTGAGGCAGGT |
| | | GGGTTTCT |
| 107 | 16:86703949_C/T | GTCTCACCCTTACCGATGTTTGGAGTTTGTAAAACCCGCTAATC |
| | | CCCGAC |
| 108 | 6:41702582_C/T | GGAGGTGTGCGGGGGCTGAGTCTGGCCAGGCCAGGGGGGGG |
| | | AGTGGGAGG |
| 109 | 6:55712124_C/T | GTCATTCTCAACCAGGGCAGTCCCAGCACTTCATTTTATGCTGT |
| | | TTGTAA |
| 110 | 8:117632965_G/C | GCTAACGGAAATACATCAACAACTACAGCTTGTGAAACACCAA |
| | | CAGTCTG |
| 111 | 1:183002639_A/G | AGGTTTAGAGAATTCTTTAAGAATGTCTTTGTGTGGCATATGCT |
| | | TTGGTC |
| 112 | 3:66365163_G/A | AAGAGTATCAGCTCCTGTTTATATTTCTGGTAGAATTCAGCTGT |
| | | AAAACC |
| 113 | 6:105966894_C/A | GGAACAACATATAATGGGGCCTGTCGGGGGGTCGGGTGGGGA |
| | | GAGGAAGA |
| 114 | 8:128413305_G/T | TGTAGCCAGAGTTAATACCCTCATCGTCCTTTGAGCTCAGCAG |
| | | ATGAAAG |
| 115 | 8:128414892_T/C | CTCCTTTCTGTTGCCTGCAGCAAAAAAGCTCCTTTGAGCAAACT |
| | | GATAGG |
| 116 | 10:80819132_A/G | GCAGCTATTTGTGTACACGGCTTATCTCCCTGCCAGACTGAGA |
| | | GGCAACG |
| 117 | 11:74280012_T/G | AGCTGCAAGTGAATCCAGCTAACAGAAATCAATATTGTCTTATT |
| | | TGAATA |
| 118 | 1:22587728_T/C | GGAACCCGAAGAGCCTTGAATGCTGGGCCGAGGAACTTCACT |
| | | CTTCATAC |
| 119 | 3:112999560_G/A | GGTGAGACACAGTTCTGTGTTTATAGCTTCTGCGTGATATAGTT |
| | | TCCTCC |
| 120 | 12:115890922_T/C | GGGAATATCACAGCCTGGGCTGTTTGGTTATCTTCATTATTAAT |
| | | ACACAC |
| 121 | 19:59079096_C/T | TGCAAGAGAGGGGCACCACAAATCTGGCTGACAGCTCTGAGC |
| | | AGGTAGAT |
| 122 | 13:37462010_A/G | ATCTGGTCACAGGATGATGAGGGTGTTCCCTCTGAAAACTCCA |
| | | AGGTTGC |
| 123 | 15:91172901_C/T | TACACTCATTATTACACCAAGCACCCATGAGGTCTCAAGTGCA |
| | | GTCTGTT |
| 124 | 1:62673037_T/C | CTCCAGAGAAGAAAAAGTGTTGATGGATGAAGGAGCAGTACTT |
| | | ACCCTGG |
| 125 | 5:112097351_A/G | TTCTGTTTAAGACATCTTTGCCTACTCCCAGGACAAGAAGCTAT |
| | | TACCTA |
| 126 | 17:81061048_A/G | GCCAGGAACCGGTGGGATTCCTGGGAATGGGGCCAGGGAGT |
| | | GGACAGAAG |
| 127 | 5:40102443_G/A | CTTCCCCTGGAGTTGGACGCCTCAGTGACTGGACTCTTCTCTG |
| | | ACCACCC |
| 128 | 13:73791554_T/C | TCCTTGGTCCCATTCCTGACTTACTTAAAATCAGAATTTCTAGG |
| | | GATGGG |
| 129 | 5:1240204_C/T | TGCATGAGATGGGAGAGGCAGGTTGGTCCCTGGGCAGCTCTG |
| | | CCCGCTGG |
| 130 | 13:111075881_C/T | TCAAAAGGTAATTTCGATGTTATGCATTTATTTTATTTTATTAGT |
| | | ACCCT |
| 131 | 14:59208437_G/A | GAAGCATGCTGAATTCACTGTAATTACAAAGGTAATAAAAATGA |
| | | GCCACG |
| 132 | 6:32593080_A/G | AATCCTTTTACATAACTTTACTCATCTCTACCTCTCCCTTCCCTC |
| | | CAAAG |
| 133 | 6:36623379_G/A | TGCTGCAGAGAGGAGACAGAACTCTAAAAAGGATGTGTGACAC |
| | | ACAGAGC |
| 134 | 3:53088285_T/G | CTCCTGATCTCAAGTGATTTGCCAGCCTCGGCCTCCCAAAGTG |
| | | CTAGGAT |
| 135 | 17:70413253_G/A | GAATGTTACCTTATGGTATATGGTAAAAGGGACTTGGCAGATG |
| | | GAATTAA |
| 136 | 2:199781586_T/C | TTCATTTTGCCACAAAGCAAGGAGGTATGTGAGTCGTTTCAAG |
| | | GAGCATG |
| 137 | 3:169517436_C/T | CGTGAGCCGATATCATGCCACCGCACTCCAGCCTGGGTGACA |
| | | GAGTGAGA |
| 138 | 16:68743939_A/C | AATGTAACCAACCTAATCCAGAACACAGGGCAGCTAGGGCTGC |
| | | CTCCTGG |
| 139 | 16:80043258_C/A | AGGTGTTTTTATAATCATACTCATGCTTCATTGTTTTTAGCAGAT |
| | | AAAGC |
| 140 | 20:6603622_C/T | ATGTGTTCAATCTTGAATCAATTACCAGAGTCCAAGTTGATGAA |
| | | GTGTTC |

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (e.g., oncology, colorectal cancer statistics, molecular genetics, bioinformatics and biochemistry).

Unless otherwise indicated, the molecular and statistical techniques utilized in the present disclosure are standard procedures, well known to those skilled in the art. Such techniques are described and explained throughout the literature in sources such as, J. Perbal, A Practical Guide to Molecular Cloning, John Wiley and Sons (1984); J. Sambrook et al., Molecular Cloning: A Laboratory Manual, Cold Spring Harbour Laboratory Press (1989); T. A. Brown (editor), Essential Molecular Biology: A Practical Approach, Volumes 1 and 2, IRL Press (1991); D. M. Glover and B. D. Hames (editors), DNA Cloning: A Practical Approach, Volumes 1-4, IRL Press (1995 and 1996); F. M. Ausubel et al. (editors), Current Protocols in Molecular Biology, Greene Pub. Associates and Wiley-Interscience (1988, including all updates until present); E. Harlow and D. Lane (editors), Antibodies: A Laboratory Manual, Cold Spring Harbour Laboratory, (1988); and J. E. Coligan et al. (editors), Current Protocols in Immunology, John Wiley & Sons (including all updates until present).

It is to be understood that this disclosure is not limited to particular embodiments, which can, of course, vary. It is also to be understood that the terminology used herein is for the purpose of describing particular embodiments only, and is not intended to be limiting. As used in this specification and the appended claims, terms in the singular and the singular forms “a,” “an” and “the,” for example, optionally include plural referents unless the content clearly dictates otherwise. Thus, for example, reference to “a probe” optionally includes a plurality of probe molecules; similarly, depending on the context, use of the term “a nucleic acid” optionally includes, as a practical matter, many copies of that nucleic acid molecule.

The term “and/or”, for example, “X and/or Y” shall be understood to mean either “X and Y” or “X or Y” and shall be taken to provide explicit support for both meanings or for either meaning.

As used herein, the term “about”, unless stated to the contrary, refers to ±10%, more preferably ±5%, more preferably ±1%, of the designated value.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein, the term “colorectal cancer” encompasses any type of cancer that can develop in the colon or rectum of a subject. The terms “colorectal cancer”, “colon cancer”, “rectal cancer” and “bowel cancer” can be used interchangeably in the context of the present disclosure. For example, the colorectal cancer may be characterised as T stage 1-4. In another example, the colorectal cancer may be characterised as Dukes stage A-D. As used herein, “colorectal cancer” also encompasses a phenotype that displays a predisposition towards developing colorectal cancer in an individual. A phenotype that displays a predisposition for colorectal cancer, can, for example, show a higher likelihood that the cancer will develop in an individual with the phenotype than in members of a relevant general population under a given set of environmental conditions (diet, physical activity regime, geographic location, etc.). For example, the colorectal cancer may be classified clinically as pre-malignant (e.g. hyperplasia, adenoma).

A “polymorphism” is a locus that is variable; that is, within a population, the nucleotide sequence at a polymorphism has more than one version or allele. One example of a polymorphism is a “single-nucleotide polymorphism”, which is a polymorphism at a single-nucleotide position in a genome (the nucleotide at the specified position varies between individuals or populations). Other examples include a deletion or insertion of one or more base pairs at the polymorphism locus.

As used herein, the term “SNP” or “single-nucleotide polymorphism” refers to a genetic variation between individuals; for example, a single nitrogenous base position in the DNA of organisms that is variable. As used herein, “SNPs” is the plural of SNP. Of course, when one refers to DNA herein, such reference may include derivatives of the DNA such as amplicons, RNA transcripts thereof, etcetera.

The term “allele” refers to one of two or more different nucleotide sequences that occur or are encoded at a specific locus, or two or more different polypeptide sequences encoded by such a locus. For example, a first allele can occur on one chromosome, while a second allele occurs on a second homologous chromosome, e.g., as occurs for different chromosomes of a heterozygous individual, or between different homozygous or heterozygous individuals in a population. An allele “positively” correlates with a trait when it is linked to it and when presence of the allele is an indicator that the trait or trait form will occur in an individual carrying the allele. An allele “negatively” correlates with a trait when it is linked to it and when presence of the allele is an indicator that a trait or trait form will not occur in an individual carrying the allele.

A marker polymorphism or allele is “correlated”, or “associated” with a specified phenotype (colorectal cancer susceptibility, etc.) when it can be statistically linked (positively or negatively) to the phenotype (also referred to herein as an “effect allele”). The non-correlated or non-associated allele can also be referred to as the “reference allele”. Methods for determining whether a polymorphism or allele is statistically linked are known to those in the art. That is, the specified polymorphism occurs more commonly in a case population (e.g., colorectal cancer patients) than in a control population (e.g., individuals who do not have colorectal cancer). This correlation is often inferred as being causal in nature, but it need not be, simple genetic linkage to (association with) a locus for a trait that underlies the phenotype is sufficient for correlation/association to occur.

The phrase “linkage disequilibrium” (LD) is used to describe the statistical correlation between two neighbouring polymorphic genotypes. Typically, LD refers to the correlation between the alleles of a random gamete at the two loci, assuming Hardy-Weinberg equilibrium (statistical independence) between gametes. LD is quantified with either Lewontin's parameter of association (D′) or with Pearson correlation coefficient (r) (Devlin and Risch, 1995). Two loci with a LD value of 1 are said to be in complete LD. At the other extreme, two loci with a LD value of 0 are said to be in linkage equilibrium. Linkage disequilibrium is calculated following the application of the expectation maximization algorithm for the estimation of haplotype frequencies (Slatkin and Excoffier, 1996). LD values according to the present disclosure for neighbouring genotypes/loci are selected above 0.1, preferably, above 0.2, more preferable above 0.5, more preferably, above 0.6, still more preferably, above 0.7, preferably, above 0.8, more preferably above 0.9, ideally about 1.0.

Another way one of skill in the art can readily identify polymorphisms in linkage disequilibrium with the polymorphisms of the present disclosure is determining the LOD score for two loci. LOD stands for “logarithm of the odds”, a statistical estimate of whether two genes, or a gene and a disease gene, are likely to be located near each other on a chromosome and are therefore likely to be inherited together. A LOD score of between about 2-3 or higher is generally understood to mean that two genes are located close to each other on the chromosome. The present inventors have found that many of the polymorphisms in linkage disequilibrium with the polymorphisms of the present disclosure have a LOD score of between about 2-50. Accordingly, in an embodiment, LOD values according to the present disclosure for neighbouring genotypes/loci are selected at least above 2, at least above 3, at least above 4, at least above 5, at least above 6, at least above 7, at least above 8, at least above 9, at least above 10, at least above 20 at least above 30, at least above 40, at least above 50.

In another embodiment, polymorphisms in linkage disequilibrium with the polymorphisms of the present disclosure can have a specified genetic recombination distance of less than or equal to about 20 centimorgan (CM) or less. For example, 15 cM or less, 10 cM or less, 9 cM or less, 8 CM or less, 7 CM or less, 6 CM or less, 5 CM or less, 4 cM or less, 3 cM or less, 2 cM or less, 1 cM or less, 0.75 cM or less, 0.5 CM or less, 0.25 cM or less, or 0.1 cM or less. For example, two linked loci within a single chromosome segment can undergo recombination during meiosis with each other at a frequency of less than or equal to about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, about 1%, about 0.75%, about 0.5%, about 0.25%, or about 0.1% or less.

In another embodiment, polymorphisms in linkage disequilibrium with the polymorphisms of the present disclosure are within at least 100 kb (which correlates in humans to about 0.1 cM, depending on local recombination rate), at least 50 kb, at least 20 kb or less of each other.

For example, one approach for the identification of surrogate markers for a particular polymorphism involves a simple strategy that presumes that polymorphisms surrounding the target polymorphism are in linkage disequilibrium and can therefore provide information about disease susceptibility. Thus, as described herein, surrogate markers can be identified from publicly available databases, such as HAPMAP, by searching for polymorphisms fulfilling certain criteria that have been found in the scientific community to be suitable for the selection of surrogate marker candidates.

“Allele frequency”, or number of a particular allele, refers to the frequency (proportion or percentage) at which an allele is present at a locus within an individual, or within a given population. For example, for an allele “A”, diploid individuals of genotype “AA”, “Aa” or “aa” (alternatively “AA”, “AB” or “BB”) have allele frequencies of 1.0, 0.5, or 0.0, respectively. One can estimate the allele frequency within a line or population (e.g., cases or controls) by averaging the allele frequencies of a sample of individuals from that line or population. Similarly, one can calculate the allele frequency within a population of lines by averaging the allele frequencies of lines that make up the population.

In an embodiment, the term “allele frequency” is used to define the population frequency of the allele of interest, which is known as the effect allele. The effect allele is that linked to colorectal cancer risk, either positively or negatively.

An individual is “homozygous” if the individual has only one type of allele at a given locus (e.g., a diploid individual has a copy of the same allele at a locus for each of two homologous chromosomes). An individual is “heterozygous” if more than one allele type is present at a given locus (e.g., a diploid individual with one copy each of two different alleles). The term “homogeneity” indicates that members of a group have the same genotype at one or more specific loci. In contrast, the term “heterogeneity” is used to indicate that individuals within the group differ in genotype at one or more specific loci.

A “locus” is a chromosomal position or region. For example, a polymorphic locus is a position or region where a polymorphic nucleic acid, trait determinant, gene or marker is located. In a further example, a “gene locus” is a specific chromosome location (region) in the genome of a species where a specific gene can be found.

A “marker”, “molecular marker” or “marker nucleic acid” refers to a nucleotide sequence or encoded product thereof (e.g., a protein) used as a point of reference when identifying a locus or a linked locus. A marker can be derived from genomic nucleotide sequence or from expressed nucleotide sequences (e.g., from an RNA, nRNA, mRNA, a cDNA, etc.), or from an encoded polypeptide. The term also refers to nucleic acid sequences complementary to or flanking the marker sequences, such as nucleic acids used as probes or primer pairs capable of amplifying the marker sequence. A “marker probe” is a nucleic acid sequence or molecule that can be used to identify the presence of a marker locus, e.g., a nucleic acid probe that is complementary to a marker locus sequence. Nucleic acids are “complementary” when they specifically hybridize in solution, e.g., according to Watson-Crick base pairing rules. A “marker locus” is a locus that can be used to track the presence of a second linked locus, e.g., a linked or correlated locus that encodes or contributes to the population variation of a phenotypic trait. For example, a marker locus can be used to monitor segregation of alleles at a locus, such as a QTL, that are genetically or physically linked to the marker locus. Thus, a “marker allele,” alternatively an “allele of a marker locus” is one of a plurality of polymorphic nucleotide sequences found at a marker locus in a population that is polymorphic for the marker locus. Each of the identified markers is expected to be in close physical and genetic proximity (resulting in physical and/or genetic linkage) to a genetic element, e.g., a QTL that contributes to the relevant phenotype. Markers corresponding to genetic polymorphisms between members of a population can be detected by methods well established in the art. These include, e.g., DNA sequencing, PCR-based sequence specific amplification methods, detection of restriction fragment length polymorphisms (RFLP), detection of isozyme markers, detection of allele-specific hybridization (ASH), detection of single-nucleotide extension, detection of amplified variable sequences of the genome, detection of self-sustained sequence replication, detection of simple sequence repeats (SSRs), detection of single-nucleotide polymorphisms (SNPs), or detection of amplified fragment length polymorphisms (AFLPs).

The term “amplifying” in the context of nucleic acid amplification is any process whereby additional copies of a selected nucleic acid (or a transcribed form thereof) are produced. Typical amplification methods include various polymerase based replication methods, including the polymerase chain reaction (PCR), ligase mediated methods such as the ligase chain reaction (LCR) and RNA polymerase based amplification (e.g., by transcription) methods.

An “amplicon” is an amplified nucleic acid, e.g., a nucleic acid that is produced by amplifying a template nucleic acid by any available amplification method (e.g., PCR, LCR, transcription, or the like).

A “gene” is one or more sequence(s) of nucleotides in a genome that together encode one or more expressed molecules, e.g., an RNA, or polypeptide. The gene can include coding sequences that are transcribed into RNA, which may then be translated into a polypeptide sequence, and can include associated structural or regulatory sequences that aid in replication or expression of the gene.

A “genotype” is the genetic constitution of an individual (or group of individuals) at one or more genetic loci. Genotype is defined by the allele(s) of one or more known loci of the individual, typically, the compilation of alleles inherited from its parents.

A “haplotype” is the genotype of an individual at a plurality of genetic loci on a single DNA strand. Typically, the genetic loci described by a haplotype are physically and genetically linked, i.e., on the same chromosome strand.

A “set” of markers, probes or primers refers to a collection or group of markers probes, primers, or the data derived therefrom, used for a common purpose, e.g., identifying an individual with a specified genotype (e.g., risk of developing colorectal cancer). Frequently, data corresponding to the markers, probes or primers, or derived from their use, is stored in an electronic medium. While each of the members of a set possess utility with respect to the specified purpose, individual markers selected from the set as well as subsets including some, but not all of the markers, are also effective in achieving the specified purpose.

The polymorphisms and genes, and corresponding marker probes, amplicons or primers described above can be embodied in any system herein, either in the form of physical nucleic acids, or in the form of system instructions that include sequence information for the nucleic acids. For example, the system can include primers or amplicons corresponding to (or that amplify a portion of) a gene or polymorphism described herein. As in the methods above, the set of marker probes or primers optionally detects a plurality of polymorphisms in a plurality of said genes or genetic loci. Thus, for example, the set of marker probes or primers detects at least one polymorphism in each of these polymorphisms or genes, or any other polymorphism, gene or locus defined herein. Any such probe or primer can include a nucleotide sequence of any such polymorphism or gene, or a complementary nucleic acid thereof, or a transcribed product thereof (e.g., an RNA or mRNA form produced from a genomic sequence, e.g., by transcription or splicing).

As used herein, “risk assessment” refers to a process by which a subject's risk of developing colorectal cancer a can be assessed. A risk assessment will typically involve obtaining information relevant to the subject's risk of developing colorectal cancer, assessing that information, and quantifying the subject's risk of developing colorectal cancer, for example, by producing a risk score.

As used herein, “centred relative risk” is calculated using the population frequency of the risk factor and the relative risk such that the population average risk is equal to 1.

As used herein, the term “combining the genetic risk assessment with the clinical risk assessment to obtain the risk” refers to any suitable mathematical analysis relying on the results of the two assessments. For example, the results of the clinical risk assessment and the genetic risk assessment may be added, more preferably multiplied.

As used herein, the terms “routinely screening for colorectal cancer” and “more frequent screening” are relative terms, and are based on a comparison to the level of screening recommended to a subject who has no identified risk of developing colorectal cancer. For example, routine screening can include fecal occult screening, or fecal immunochemical test every year, multi-targeted stool DNA test every three years, colonoscopy every 10 years, CT colonoscopy or flexible sigmoidoscopy every five years. Various other time intervals for routine screening are discussed below.

### Genetic Risk Factors

In an embodiment, the methods of the present disclosure relate to assessing the risk of a subject for developing colorectal cancer by performing a genetic risk assessment.

Various exemplary polymorphisms associated with colorectal cancer are discussed in the present disclosure. These polymorphisms vary in terms of penetrance and many would be understood by those of skill in the art to be low penetrance polymorphisms.

The term “penetrance” is used in the context of the present disclosure to refer to the extent to which a particular polymorphism is present within subjects with colorectal cancer as opposed to those without. “High penetrance” polymorphisms will often be apparent in a subject with colorectal cancer and are considered rare (with a population frequency less than 1%), and thus labelled a variant as opposed to a polymorphism (these variants have a much greater odds ratio associated with colorectal cancer, greater than 1.5, greater than 2) while “low penetrance” polymorphisms will only sometimes be apparent in a subject with colorectal cancer because they are more common in the population (population frequency greater than 1% and an odds ratio less than 1.5). In an embodiment, polymorphisms assessed as part of a genetic risk assessment according to the present disclosure are low penetrance polymorphisms.

The genetic risk assessment is performed by analysing the genotype of the subject at 50 or more loci for single nucleotide polymorphisms. For example, at least 50, at least 75, at least 100, at least 120, at least 130, at least 135, or each of the polymorphisms selected from Table 1 or a polymorphism in linkage disequilibrium with one or more thereof.

TABLE 1
Panel of 140 single-nucleotide polymorphisms (Thomas et al., 2020).

| | | | | | | Effect | |
|---|---|---|---|---|---|---|---|
| | | | | GRCh37 | Effect | allele | |
| Locus | RS ID | Variant | Chromosome | position | allele | frequency | Beta |
| 1p34.3 | rs4360494 | 1:38455891_G/C | 1 | 38455891 | G | 0.4539 | 0.0379 |
| 1p32.3 | rs12144319 | 1:55246035_T/C | 1 | 55246035 | C | 0.2548 | 0.0661 |
| 1p36.12 | rs72647484 | 1:22587728_T/C | 1 | 22587728 | T | 0.9107 | 0.0504 |
| 1p31.3 | rs7542665 | 1:62673037_T/C | 1 | 62673037 | C | 0.273 | 0.0334 |
| 1q25.3 | rs6678517 | 1:183002639_A/G | 1 | 183002639 | A | 0.5898 | 0.073 |
| 1q41 | rs17011141 | 1:222112634_A/G | 1 | 222112634 | G | 0.2087 | 0.0877 |
| 2q24.2 | rs448513 | 2:159964552_T/C | 2 | 159964552 | C | 0.326 | 0.0054 |
| 2q33.1 | rs11884596 | 2:199612407_T/C | 2 | 199612407 | C | 0.3823 | 0.0342 |
| 2q33.1 | rs983402 | 2:199781586_T/C | 2 | 199781586 | T | 0.3312 | 0.0622 |
| 2p16.3 | rs7606562 | 2:48686695_T/A | 2 | 48686695 | T | 0.813 | 0.0414 |
| 2q11.2 | rs11692435 | 2:98275354_G/A | 2 | 98275354 | G | 0.9 | 0.0492 |
| 2q35 | rs3731861 | 2:219191256_T/C | 2 | 219191256 | T | 0.6295 | 0.0613 |
| 3q22.2 | rs10049390 | 3:133701119_G/A | 3 | 133701119 | A | 0.7353 | 0.0455 |
| 3q13.2 | rs13086367 | 3:112903888_A/G | 3 | 112903888 | A | 0.5262 | 0.0463 |
| 3q13.2 | rs72942485 | 3:112999560_G/A | 3 | 112999560 | G | 0.9802 | 0.0545 |
| 3p21.1 | rs9831861 | 3:53088285_T/G | 3 | 53088285 | G | 0.59 | 0.0294 |
| 3p22.1 | rs35470271 | 3:40915239_A/G | 3 | 40915239 | G | 0.154 | 0.0994 |
| 3q13.2 | rs12635946 | 3:112916918_C/T | 3 | 112916918 | C | 0.62 | 0.0334 |
| 3q22.2 | rs113569514 | 3:133748789_T/C | 3 | 133748789 | T | 0.62 | 0.0414 |
| 3q26.2 | rs9876206 | 3:169517436_C/T | 3 | 169517436 | C | 0.7507 | 0.0453 |
| 3p14.1 | rs6781752 | 3:66365163_G/A | 3 | 66365163 | A | 0.205 | 0.0597 |
| 4q31.21 | rs11727676 | 4:145659064_T/C | 4 | 145659064 | C | 0.098 | 0.0093 |
| 4q24 | rs1391441 | 4:106128760_G/A | 4 | 106128760 | A | 0.672 | 0.0148 |
| 4q22.2 | rs13149359 | 4:94938618_C/A | 4 | 94938618 | A | 0.3663 | 0.052 |
| 5p13.1 | rs7708610 | 5:40102443_G/A | 5 | 40102443 | A | 0.3564 | 0.0384 |
| 5p15.33 | rs78368589 | 5:1240204_C/T | 5 | 1240204 | T | 0.0597 | 0.0786 |
| 5q21.1 | rs145364999 | 5:98206082_T/A | 5 | 98206082 | T | 0.9969 | 0.3496 |
| 5p15.33 | rs2735940 | 5:1296486_A/G | 5 | 1296486 | G | 0.4952 | 0.0865 |
| 5p13.1 | rs12514517 | 5:40280076_G/A | 5 | 40280076 | A | 0.288 | 0.1013 |
| 5q22.2 | rs755229494 | 5:112097351_A/G | 5 | 112097351 | G | 0.0011 | 0.6286 |
| 5q23.2 | rs12659017 | 5:125988175_G/A | 5 | 125988175 | G | 0.232 | 0.0374 |
| 5q31.1 | rs4976270 | 5:134467220_C/T | 5 | 134467220 | C | 0.5501 | 0.0693 |
| 6p12.1 | rs13204733 | 6:55566108_A/G | 6 | 55566108 | G | 0.141 | 0.0643 |
| 6p21.33 | rs116685461 | 6:31315512_G/A | 6 | 31315512 | G | 0.8755 | 0.0655 |
| 6p21.32 | rs9271695 | 6:32593080_A/G | 6 | 32593080 | G | 0.7954 | 0.0889 |
| 6p21.33 | rs2516420 | 6:31449620_C/T | 6 | 31449620 | C | 0.9263 | 0.1091 |
| 6p21.33 | rs116353863 | 6:31010185_T/C | 6 | 31010185 | C | 0.0165 | 0.1202 |
| 6p21.31 | rs16878812 | 6:35569562_A/G | 6 | 35569562 | A | 0.8861 | 0.0778 |
| 6p21.2 | rs9470361 | 6:36623379_G/A | 6 | 36623379 | A | 0.2488 | 0.054 |
| 6p12.1 | rs62404966 | 6:55712124_C/T | 6 | 55712124 | C | 0.7623 | 0.0724 |
| 6p21.33 | rs3131043 | 6:30758466_A/G | 6 | 30758466 | G | 0.43 | 0.0294 |
| 6p24.1 | rs2070699 | 6:12292772_G/T | 6 | 12292772 | T | 0.48 | 0.0294 |
| 6p22.1 | rs1476570 | 6:29809860_G/A | 6 | 29809860 | A | 0.376 | 0.0492 |
| 6p21.32 | rs3830041 | 6:32191339_C/T | 6 | 32191339 | T | 0.14 | 0.0645 |
| 6q21 | rs6928864 | 6:105966894_C/A | 6 | 105966894 | C | 0.91 | 0.0531 |
| 6p21.1 | rs62396735 | 6:41702582_C/T | 6 | 41702582 | C | 0.2908 | 0.033 |
| 7p13 | rs12672022 | 7:45136423_T/C | 7 | 45136423 | T | 0.8345 | 0.0067 |
| 7p12.3 | rs80077929 | 7:46094089_C/T | 7 | 46094089 | T | 0.1107 | 0.0093 |
| 7p12.3 | rs10951878 | 7:46926695_C/T | 7 | 46926695 | C | 0.91 | 0.0531 |
| 7p12.3 | rs3801081 | 7:47511161_A/G | 7 | 47511161 | G | 0.49 | 0.0253 |
| 8q24.21 | rs7013278 | 8:128414892_T/C | 8 | 128414892 | T | 0.3761 | 0.0091 |
| 8q24.21 | rs4313119 | 8:128571855_G/T | 8 | 128571855 | G | 0.7486 | 0.0518 |
| 8q23.3 | rs16892766 | 8:117630683_A/C | 8 | 117630683 | C | 0.0829 | 0.2099 |
| 8q23.3 | rs6469654 | 8:117632965_G/C | 8 | 117632965 | G | 0.2288 | 0.0677 |
| 8q24.11 | rs117079142 | 8:117790914_C/A | 8 | 117790914 | A | 0.0432 | 0.1139 |
| 8q24.21 | rs6983267 | 8:128413305_G/T | 8 | 128413305 | G | 0.5228 | 0.1052 |
| 9q22.33 | rs34405347 | 9:101679752_T/G | 9 | 101679752 | T | 0.9034 | 0.0089 |
| 9p21.3 | rs1537372 | 9:22103183_G/T | 9 | 22103183 | G | 0.5692 | 0.012 |
| 9q31.3 | rs10980628 | 9:113671403_T/C | 9 | 113671403 | C | 0.2106 | 0.0511 |
| 10p14 | rs12217641 | 10:8663875_C/T | 10 | 8663875 | C | 0.6981 | 0.0069 |
| 10q24.2 | rs10786560 | 10:101315166_G/A | 10 | 101315166 | G | 0.762 | 0.0082 |
| 10q22.3 | rs1250567 | 10:81046265_T/C | 10 | 81046265 | C | 0.4405 | 0.047 |
| 10p14 | rs11255841 | 10:8739580_T/A | 10 | 8739580 | T | 0.703 | 0.1064 |
| 10q11.23 | rs10821907 | 10:52648454_C/T | 10 | 52648454 | C | 0.8276 | 0.073 |
| 10q22.3 | rs704017 | 10:80819132_A/G | 10 | 80819132 | G | 0.5846 | 0.0765 |
| 10q24.2 | rs11190164 | 10:101351704_A/G | 10 | 101351704 | G | 0.2626 | 0.0889 |
| 10q25.2 | rs12246635 | 10:114288619_T/C | 10 | 114288619 | C | 0.0983 | 0.0975 |
| 10q25.2 | rs11196170 | 10:114722621_G/A | 10 | 114722621 | A | 0.2178 | 0.0527 |
| 11q13.4 | rs7946853 | 11:74409077_T/C | 11 | 74409077 | C | 0.8624 | 0.0119 |
| 11q22.1 | rs55864876 | 11:100717136_G/A | 11 | 100717136 | G | 0.9184 | 0.015 |
| 11q22.1 | rs2186607 | 11:101656397_T/A | 11 | 101656397 | T | 0.5178 | 0.0483 |
| 11q13.4 | rs61389091 | 11:74427921_C/T | 11 | 74427921 | C | 0.9606 | 0.1934 |
| 11p15.4 | rs4450168 | 11:10286755_A/C | 11 | 10286755 | C | 0.17 | 0.0413 |
| 11q12.2 | rs174533 | 11:61549025_G/A | 11 | 61549025 | G | 0.6739 | 0.0636 |
| 11q13.4 | rs7121958 | 11:74280012_T/G | 11 | 74280012 | G | 0.5105 | 0.078 |
| 11q23.1 | rs3087967 | 11:111156836_T/C | 11 | 111156836 | T | 0.2911 | 0.1122 |
| 12q13.3 | rs4759277 | 12:57533690_C/A | 12 | 57533690 | A | 0.3546 | 0.0285 |
| 12q24.21 | rs1427760 | 12:115100714_T/C | 12 | 115100714 | C | 0.5268 | 0.0424 |
| 12p13.32 | rs3217874 | 12:4400808_C/T | 12 | 4400808 | T | 0.4282 | 0.0453 |
| 12p13.31 | rs10849433 | 12:6406904_T/C | 12 | 6406904 | C | 0.267 | 0.0468 |
| 12q12 | rs11610543 | 12:43134191_A/G | 12 | 43134191 | G | 0.5013 | 0.0474 |
| 12p13.32 | rs35808169 | 12:4368607_T/C | 12 | 4368607 | C | 0.1721 | 0.089 |
| 12p13.32 | rs3217810 | 12:4388271_C/T | 12 | 4388271 | T | 0.1253 | 0.1181 |
| 12p13.31 | rs2250430 | 12:6421174_A/T | 12 | 6421174 | T | 0.7095 | 0.0597 |
| 12p11.21 | rs77969132 | 12:31594813_C/T | 12 | 31594813 | T | 0.015 | 0.1583 |
| 12q13.12 | rs12372718 | 12:51171090_A/G | 12 | 51171090 | G | 0.3924 | 0.0896 |
| 12q24.12 | rs597808 | 12:111973358_A/G | 12 | 111973358 | G | 0.5166 | 0.0737 |
| 12q24.21 | rs7300312 | 12:115890922_T/C | 12 | 115890922 | C | 0.5719 | 0.066 |
| 12p13.2 | rs2710310 | 12:12035649_C/T | 12 | 12035649 | C | 0.7596 | 0.0145 |
| 13q22.1 | rs78341008 | 13:73791554_T/C | 13 | 73791554 | C | 0.0719 | 0.0109 |
| 13q34 | rs8000189 | 13:111075881_C/T | 13 | 111075881 | T | 0.6401 | 0.0473 |
| 13q22.1 | rs45597035 | 13:73649152_A/G | 13 | 73649152 | A | 0.6506 | 0.0495 |
| 13q22.1 | rs1924816 | 13:73997961_A/G | 13 | 73997961 | A | 0.7737 | 0.0506 |
| 13q13.3 | rs7333607 | 13:37462010_A/G | 13 | 37462010 | G | 0.235 | 0.0758 |
| 13q22.3 | rs1330889 | 13:78609615_T/C | 13 | 78609615 | C | 0.87 | 0.0453 |
| 13q13.2 | rs9537756 | 13:34092164_C/T | 13 | 34092164 | C | 0.6117 | 0.0468 |
| 14q22.2 | rs1951864 | 14:54369299_G/A | 14 | 54369299 | A | 0.3722 | 0.0059 |
| 14q23.1 | rs17094983 | 14:59189361_G/A | 14 | 59189361 | G | 0.8773 | 0.0062 |
| 14q23.1 | rs8020436 | 14:59208437_G/A | 14 | 59208437 | A | 0.4016 | 0.0294 |
| 14q22.2 | rs35107139 | 14:54419106_A/C | 14 | 54419106 | C | 0.4235 | 0.0912 |
| 14q22.2 | rs4901473 | 14:54445157_G/A | 14 | 54445157 | G | 0.378 | 0.0465 |
| 15q23 | rs745213 | 15:68060389_T/G | 15 | 68060389 | G | 0.8102 | 0.0072 |
| 15q22.31 | rs12594720 | 15:67007018_C/G | 15 | 67007018 | C | 0.7218 | 0.0246 |
| 15q22.33 | rs56324967 | 15:67402824_T/C | 15 | 67402824 | C | 0.6757 | 0.0689 |
| 15q13.3 | rs17816465 | 15:33156386_G/A | 15 | 33156386 | A | 0.2055 | 0.069 |
| 15q13.3 | rs12708491 | 15:32992836_G/A | 15 | 32992836 | G | 0.5872 | 0.0464 |
| 15q13.3 | rs2293581 | 15:33010736_G/A | 15 | 33010736 | A | 0.2116 | 0.1248 |
| 15q26.1 | rs7495132 | 15:91172901_C/T | 15 | 91172901 | T | 0.12 | 0.0453 |
| 16q23.2 | rs9930005 | 16:80043258_C/A | 16 | 80043258 | C | 0.4303 | 0.0061 |
| 16q24.1 | rs12447408 | 16:86252544_G/A | 16 | 86252544 | A | 0.2535 | 0.0079 |
| 16q22.1 | rs9924886 | 16:68743939_A/C | 16 | 68743939 | A | 0.7321 | 0.055 |
| 16q24.1 | rs12149163 | 16:86339315_T/C | 16 | 86339315 | T | 0.4976 | 0.0487 |
| 16q24.1 | rs62042090 | 16:86703949_C/T | 16 | 86703949 | T | 0.2164 | 0.0481 |
| 17q24.3 | rs983318 | 17:70413253_G/A | 17 | 70413253 | A | 0.2526 | 0.0397 |
| 17p13.3 | rs73975586 | 17:814243_A/T | 17 | 814243 | A | 0.8732 | 0.0497 |
| 17p12 | rs1078643 | 17:10707241_G/A | 17 | 10707241 | A | 0.7636 | 0.0747 |
| 17q25.3 | rs75954926 | 17:81061048_A/G | 17 | 81061048 | G | 0.6568 | 0.0882 |
| 17q25.3 | rs373585858 | 17:80394556_G/A | 17 | 80394556 | A | 0.0016 | 0.1103 |
| 17p13.3 | rs4968127 | 17:809643_G/A | 17 | 809643 | G | 0.3684 | 0.0514 |
| 18q21.1 | rs11874392 | 18:46453156_A/T | 18 | 46453156 | A | 0.545 | 0.1606 |
| 19q13.43 | rs73068325 | 19:59079096_C/T | 19 | 59079096 | T | 0.1826 | 0.0066 |
| 19p13.11 | rs34797592 | 19:16417198_C/T | 19 | 16417198 | T | 0.1182 | 0.0824 |
| 19q13.11 | rs28840750 | 19:33519927_T/G | 19 | 33519927 | T | 0.948 | 0.1939 |
| 19q13.2 | rs1963413 | 19:41871573_G/A | 19 | 41871573 | A | 0.6119 | 0.0441 |
| 19q13.33 | rs12979278 | 19:49218602_C/T | 19 | 49218602 | T | 0.53 | 0.0293 |
| 20q13.33 | rs2738783 | 20:62308612_T/G | 20 | 62308612 | T | 0.2029 | 0.006 |
| 20q13.13 | rs6067417 | 20:48983697_C/T | 20 | 48983697 | C | 0.5635 | 0.0331 |
| 20q13.12 | rs6031311 | 20:42666475_C/T | 20 | 42666475 | T | 0.7591 | 0.0362 |
| 20q13.13 | rs6091189 | 20:49256285_C/T | 20 | 49256285 | T | 0.1529 | 0.0549 |
| 20p12.3 | rs994308 | 20:6603622_C/T | 20 | 6603622 | C | 0.5939 | 0.0626 |
| 20p12.3 | rs28488 | 20:6762221_C/T | 20 | 6762221 | T | 0.6388 | 0.0714 |
| 20p12.3 | rs556532366 | 20:8568071_C/T | 20 | 8568071 | T | 0.0029 | 0.0715 |
| 20p12.3 | rs189583 | 20:6376457_G/C | 20 | 6376457 | G | 0.3298 | 0.0795 |
| 20p12.3 | rs4813802 | 20:6699595_T/G | 20 | 6699595 | G | 0.3561 | 0.0819 |
| 20p12.3 | rs11087784 | 20:7740976_A/G | 20 | 7740976 | G | 0.1523 | 0.0874 |
| 20q13.13 | rs6066825 | 20:47340117_A/G | 20 | 47340117 | A | 0.6448 | 0.0719 |
| 20q13.13 | rs6063514 | 20:49055318_C/T | 20 | 49055318 | C | 0.6086 | 0.0547 |
| 20q13.32 | rs13831 | 20:57475191_A/G | 20 | 57475191 | G | 0.684 | 0.0334 |
| 20q13.33 | rs1741640 | 20:60932414_T/C | 20 | 60932414 | C | 0.7652 | 0.1146 |
| 20q11.22 | rs6058093 | 20:33213196_A/C | 20 | 33213196 | C | 0.4942 | 0.045 |

Note:
GRCh37, Genome Reference Consortium human build 37.

In an example, single nucleotide polymorphisms in linkage disequilibrium with one or more of the single nucleotide polymorphisms selected from Table 1 have LD values of at least 0.5, at least 0.6, at least 0.7, at least 0.8. In another example, single nucleotide polymorphisms in linkage disequilibrium have LD values of at least 0.9. In another example, single nucleotide polymorphisms in linkage disequilibrium have LD values of at least 1.

SNPs in linkage disequilibrium with those specifically mentioned herein are easily identified by those of skill in the art and are used to infer genotypes. Although all SNP in Table 1 can be imputed if need be, the following SNP are imputed regularly; 10:101315166_G/A, 12:6406904_T/C, 6:31010185_T/C, 6:31315512_G/A, 10:8663875_C/T, 16:86252544_G/A, 10:81046265_T/C, 15:67007018_C/G, 5:125988175_G/A, 6:55566108_A/G, 6:29809860_G/A, 13:73997961_A/G, 14:54369299_G/A, 17:80394556_G/A, 13:34092164_C/T, 13:73649152_A/G, 20:8568071_C/T, 11:100717136_G/A, 17:814243_A/T, 15:68060389_T/G, 2:48686695_T/A, 12:31594813_C/T, 11:74409077_T/C and 7:46094089_C/T.

### Clinical Risk Assessment

Clinical information can be self-reported by the subject. For example, the subject may complete a questionnaire designed to obtain information regarding the clinical risk factors. In another example, after obtaining informed consent from the subject, clinical information could be obtained from medical records by interrogating a relevant database comprising the clinical information.

Any suitable clinical risk assessment procedure can be used in the present disclosure. Preferably, the clinical risk assessment does not involve genotyping the subject at one or more loci. Nonetheless, the clinical risk assessment procedure may include obtaining information on mutations in the MLH1, MSH2 and MSH6 genes and microsatellite instability status.

In another embodiment, the clinical risk assessment procedure includes obtaining information from the subject on one or more of the following: medical history of colorectal cancer and/or polyps, age, family history of colorectal cancer and/or polyps and/or other cancer including the age of the relative at the time of diagnosis, results of previous colonoscopy and/or sigmoidoscopy, results of previous faecal occult blood test, previous biopsy status, weight, body mass index, height, sex, alcohol consumption history, smoking history, exercise history, diet (e.g. consumption of folate, vegetables, red meat, fruits, fibre, and saturated fats), has the subject ever smoked, time since last colorectal cancer screen, blood triglyceride levels, prevalence of inflammatory bowel disease, race/ethnicity, aspirin and other nonsteroidal anti-inflammatory drug (NSAID) use, implementation of estrogen replacement and use of oral contraceptives. For example, the clinical risk assessment procedure can include obtaining information from the subject on any first-degree relative's history of colorectal cancer. In another example, the clinical risk assessment procedure includes obtaining information from the subject on age and/or first-degree relative's history of colorectal cancer.

In an embodiment, the clinical risk assessment includes details regarding the family history of colorectal cancer of at least some, preferably all, first-degree relatives.

In an embodiment, the clinical risk assessment involves obtaining information from the subject on first-degree relatives' history of colorectal cancer, has the subject ever smoked, has the subject had a colorectal cancer screen in the last 10 years, blood triglyceride levels and body mass index. Examples of such screening processes would be understood by the skilled person and include colonoscopy and the fecal immunochemical test (Rex et al., 2017).

In an embodiment, the subject is female and the clinical risk assessment involves obtaining information from the subject on first-degree relatives' history of colorectal cancer, has the subject ever smoked, has the subject had a colorectal cancer screen in the last 10 years and blood triglyceride levels.

In an embodiment, the subject is male and the clinical risk assessment involves obtaining information from the subject on first-degree relatives' history of colorectal cancer, has the subject ever smoked, has the subject had a colorectal cancer screen in the last 10 years and body mass index.

In an embodiment, the clinical risk assessment comprises determining whether any of the subject's first-degree relatives have, or have had, colorectal cancer. In an embodiment, the clinical risk assessment comprises determining if the subject has no first-degree relatives who have, or who have had, colorectal cancer, or whether the subject has one or more first-degree relatives who have, or who have had, colorectal cancer. In an embodiment, the centred relative risk (calculated using the population frequency of the risk factor and the relative risk such that the population average risk is equal to 1) for the history of the subjects first-degree relatives who have, or who have had, colorectal cancer is used for the clinical assessment such as provided in Table 2.

TABLE 2
First-degree family history risks of colorectal
cancer from Gafni et al (2021).

| Number of affected | Relative risk from | Centred relative risk |
|---|---|---|
| relatives | Roos et al (2019) | used in model |
| 0 | 1 | 0.92 |
| ≥1 | 2.25 | 2.10 |

In an embodiment, the clinical risk assessment if the subject has no first-degree relatives who have, or who have had, colorectal cancer (fh_rr) is 0.87 to 0.97, about 0.92 or is 0.92.

In an embodiment, the clinical risk assessment if the subject has one or more first-degree relatives who have, or who have had, colorectal cancer (fh_rr) is 1.9 to 2.3, 2 to 2.2, about 2.1 or is 2.1.

In an embodiment, the triglyceride level is the mmol/L of triglyceride in the blood of the subject.

In an embodiment, the clinical risk assessment comprises obtaining information from the subject on their body mass index (bmi). In an embodiment, the subject's bmi is their weight in kilograms divided by their height in metres squared (kg/m²). In an embodiment, the subject's bmi is the natural log of their weight in kilograms divided by their height in metres squared (natural log of kg/m²).

In another embodiment, performing the clinical risk assessment uses a model that calculates the absolute risk of developing colorectal cancer. In an embodiment, the clinical risk assessment provides a 5-year absolute risk of developing colorectal cancer. In another embodiment, the clinical risk assessment provides a 10-year absolute risk of developing colorectal cancer.

Examples of clinical risk assessment procedures include, but are not limited to, the Harvard Cancer Risk Index, the National Cancer Institute's Colorectal Cancer Risk Assessment Tool, the Cleveland Clinic Tool, the Mismatch Repair probability model (also known as MMRpro), Colorectal Risk Prediction Tool (CRiPT) and the like (see, for example, Usher-Smith et al., 2015). A wide body of research, focused on high-risk mutations and phenotypic risk factors have been compiled into these exemplary risk prediction algorithms.

The Harvard Cancer Risk Index predicts a 10-year risk of developing colorectal cancer using family history data (first-degree relatives with colorectal cancer), and environmental factors such as body mass index, aspirin use, cigarette smoking, history of inflammatory bowel disease, height, physical activity, estrogen replacement, use of oral contraceptives, and consumption of folate, vegetables, alcohol, red meat, fruits, fibre, and saturated fats. In an example, the clinical risk assessment procedure uses the Harvard Cancer Risk Index to predict the 10-year risk of the subject developing colorectal cancer.

The Colorectal Cancer Risk Assessment Tool predicts 5-, 10-, 20-year, and lifetime risks of developing colorectal cancer for people over 50 years of age based on age, sex, use of sigmoidoscopy and/or colonoscopy, current leisure time activity, use of aspirin and other NSAIDs, history of cigarette smoking, body mass index, history of hormone replacement, and consumption of vegetables. In an example, the clinical risk assessment procedure uses the Colorectal Cancer Risk Assessment Tool to predict the 5-year risk of the subject developing colorectal cancer. In another example, the clinical risk assessment procedure uses the Colorectal Cancer Risk Assessment Tool to predict the 10-year risk of the subject developing colorectal cancer. In another example, the clinical risk assessment procedure uses the Colorectal Cancer Risk Assessment Tool to predict the 20-year risk of the subject developing colorectal cancer. In another example, the clinical risk assessment procedure uses the Colorectal Cancer Risk Assessment Tool to predict the lifetime risk of the subject developing colorectal cancer.

The Cleveland Clinic Tool provides a colorectal cancer risk score based on age, sex, ethnicity, weight, height, use of sigmoidoscopy and/or colonoscopy, faecal occult blood test, cigarette smoking, exercise, history of colorectal cancer and polyps, and consumption of vegetables and fruits.

The MMRpro model predicts five year and lifetime risks of developing colorectal and endometrial cancer based on mutations in the MLH1, MSH2 and MSH6 genes, as well as environmental factors such as family history of the disease, microsatellite instability status, age, and ethnicity. In an example, the clinical risk assessment procedure uses the MMRpro model to predict the 5-year risk of the subject developing colorectal cancer. In another example, the clinical risk assessment procedure uses the MMRpro model to predict the lifetime risk of the subject developing colorectal cancer.

The Colorectal Risk Prediction Tool (CRIPT) model uses multi-generational family history using a mixed major gene polygenic model to estimate colorectal cancer risk.

### Genetic Risk Assessment

In an embodiment, the genetic risk assessment involves determining a polygenic risk score for the subject (also referred to herein as PRS or “genetic risk score”). An individual's PRS can be defined as the weighted sum of the individuals' genotypes at multiple genetic loci. In other words, they are the linear combinations of the effect alleles across a set of candidate polymorphisms.

An individual's “genetic risk” can be defined as the product of genotype relative risk values for each SNP assessed. A log-additive risk model can then be used to define three genotypes AA, AB, and BB for a single SNP having relative risk values of 1, OR, and OR², under a rare disease model, where OR is the previously reported disease odds ratio for the effect allele, B, vs the reference allele, A. If the B allele has frequency (p), then these genotypes have population frequencies of (1−p)², 2p(1−p), and p², assuming Hardy-Weinberg equilibrium. The genotype relative risk values for each SNP can then be scaled so that based on these frequencies the average relative risk in the population is 1. Specifically, the unscaled population average relative risk for each SNP is calculated using:

$$μ = {( 1 - p )}^{2} + 2 p ( 1 - p ) OR + {p}^{2} {OR}^{2}$$

where OR is the odds ratio per effect allele and p is the effect allele frequency.

For each individual, the adjusted risk (which has a population mean equal to 1) for each of the SNPs is calculated as:

$$\frac{{OR}^{N}}{μ} ,$$

where N is the individual's number of effect alleles for the SNP.

Any individual who is missing genotype data for one or more SNPs is given an adjusted risk of 1 for each missing SNP.

The polygenic relative risk score (prs_rr) for each participant is the product of their adjusted risks for the SNPs.

In another embodiment, raw polygenic risk score (PRS) is calculated as the weighted sum of the effect allele counts for the SNPs in the PRS:

$${\sum }_{j = 1}^{p} {β}_{j} {G}_{ij}$$

where βⱼ is the weight for SNP j, Gᵢⱼ is the count (0, 1, 2) of the effect alleles of SNP j for individual i, and p is the number of SNPs in the PRS. The β weights are given in Table 1.

The PRS was standardised to have a mean of 0 and standard deviation of 1 by subtracting the population mean and dividing by the population standard deviation:

$$standardised PRS = \frac{P R {S}_{raw} - P R {S}_{\overline{x}}}{P R {S}_{sd}} ,$$

where PRS_{raw} is the individual's raw PRS, PRS_{x̅} is the population mean of PRS_{raw}, and PRS_{sd} is the population standard deviation of PRS_{raw}.

In these calculations, PRS_{x̅}=8.018 and PRS_{sd}=0.473 but these numbers will change for different populations.

In an embodiment, PRS_{x̅} is 6 to 10, 7 to 9, about 8.018 or is 8.018.

In an embodiment, PRS_{sd} is 0.273 to 0.673, 0.373 to 0.573, about 0.473 or is 0.473.

It is envisaged that the “risk” of a human subject for developing colorectal cancer can be provided as a relative risk (or risk ratio) or an absolute risk as required.

In an embodiment, the genetic risk assessment obtains the “relative risk” of a human subject for developing colorectal cancer. Relative risk (or risk ratio), measured as the incidence of a disease in individuals with a particular characteristic (or exposure) divided by the incidence of the disease in individuals without the characteristic, indicates whether that exposure increases or decreases risk. Relative risk is helpful to identify characteristics that are associated with a disease, but by itself is not particularly helpful in guiding screening decisions because the frequency of the risk (incidence) is cancelled out.

In another embodiment, the genetic risk assessment obtains the “absolute risk” of a human subject for developing colorectal cancer. Absolute risk is the numerical probability of a human subject developing colorectal cancer within a specified period (e.g. 5, 10, 15, 20 or more years). It reflects a human subject's risk of developing colorectal cancer insofar as it does not consider various risk factors in isolation.

### Combined Clinical Risk Factors and Genetic Risk

As the skilled person would be aware, in view of the teachings of the present disclosure, a variety of different formulae could be produced to provide a risk score.

In an embodiment, the genetic risk assessment is combined with the clinical risk assessment to obtain the “relative risk” of a human subject for developing colorectal cancer. In another embodiment, the genetic risk assessment is combined with the clinical risk assessment and population incidence rates to obtain the “absolute risk” of a human subject for developing colorectal cancer.

In an embodiment, the clinical and genetic relative risk assessments are combined for a female subject by determining:

$$R {R}_{fhprs \_ w} = {e}^{( PCDE 1 \times prs ) + ( PDCE 2 \times deg 1 )}$$

here:
PDCE1 is a predetermined β coefficient for the genetic risk assessment for a female subject,
PDCE2 is a predetermined β coefficient for a female subject who has at least one first-degree relative who has, or has had, colorectal cancer, and
deg1 identifies whether the subject has one or more first-degree relatives who have, or who have had, colorectal cancer.

In an embodiment, PDCE1 is 0.215 to 0.615, 0.315 to 0.515, about 0.415 or is 0.415.

In an embodiment, PDCE2 is 0.172 to 0.212, 0.82 to 0.202, about 0.192 or is 0.192.

In an embodiment, the clinical and genetic relative risk assessments are combined for a male subject by determining:

$$R {R}_{fhprs \_ w} = {e}^{( PCDE 3 \times prs ) + ( PDCE 4 \times deg 1 )}$$

where:
PDCE3 is a predetermined β coefficient for the genetic risk assessment for a male subject,
PDCE4 is a predetermined β coefficient for a male subject who has at least one first-degree relative who has, or has had, colorectal cancer, and
deg1 identifies whether the subject has one or more first-degree relatives who have, or who have had, colorectal cancer.

In an embodiment, PDCE3 is 0.200 to 0.600, 0.300 to 0.500, about 0.400 or is 0.400.

In an embodiment, PDCE4 is 0.019 to 0.519, 0.219 to 0.419, about 0.319 or is 0.319.

In an embodiment, the clinical and genetic relative risk assessments are combined for a female subject by determining:

$$R {R}_{multi \_ w} = {e}^{( PDCE 5 \times prs ) + ( PDCE 6 \times deg 1 ) + ( PDCE 7 \times smoke ) + ( PDCE 8 \times screen ) + ( PDCE 9 \times ( trigly - 3.296 ) )}$$

where:
PDCE5 is a predetermined β coefficient for the genetic risk assessment for a female subject,
PDCE6 is a predetermined β coefficient for a female subject who has at least one first-degree relative who has, or has had, colorectal cancer,
PDCE7 is a predetermined β coefficient for a female subject who has been, or is, a smoker,
PDCE8 is a predetermined β coefficient for a female subject who has had a colorectal cancer screen in the past 10 years,
PDCE9 is a predetermined β coefficient for a female subject's triglyceride levels (mmol/L),
deg1 is if the female subject has one or more first-degree relatives who have, or who have had, colorectal cancer
smoke is if the female subject has ever smoked,
screen is if the female subject has had a colorectal screen in the last, for example, 10 years, and
trigly is the female subject's blood triglyceride level in mmol/L.

In an embodiment, PDCE5 is 0.216 to 0.616, 0.316 to 0.516, about 0.416 or is 0.416.

In an embodiment, PDCE6 is 0.199 to 0.239, 0.209 to 0.229, about 0.219 or is 0.219.

In an embodiment, PDCE7 is 0.193 to 0.233, 0.203 to 0.223, about 0.213 or is 0.213.

In an embodiment, PDCE8 is −0.321 to −0.721, −0.421 to −0.621, about −0.521 or is −0.521.

In an embodiment, PDCE9 is 0.030 to 0.150, 0.060 to 0.120, about 0.090 or is 0.090.

In an embodiment, the clinical and genetic relative risk assessments are combined for a male subject by determining:

$$R {R}_{multi \_ m} = {e}^{( PDCE 10 \times prs ) + ( PDCE 11 \times deg 1 ) + ( PDCE 12 \times smoke ) + ( PDCE 13 \times screen ) + ( PDCE 14 \times ( bmi - 3.296 ) )}$$

where:
PDCE10 is a predetermined β coefficient for the genetic risk assessment for a male subject,
PDCE11 is a predetermined β coefficient for a male subject who has at least one first-degree relative who has, or has had, colorectal cancer,
PDCE12 is a predetermined β coefficient for a male subject who has been, or is, a smoker,
PDCE13 is a predetermined β coefficient for a male subject who has had a colorectal cancer screen in the past 10 years,
PDCE14 is a predetermined β coefficient for a male subject's body mass index (natural log of kg/m²),
deg1 is if the male subject has one or more first-degree relatives who have, or who have had, colorectal cancer,
smoke is if the male subject has ever smoked,
screen is if the male subject has had a colorectal screen in the last, for example, 10 years, and
bmi is the subject's body mass index expressed as the natural log of kg/m².

In an embodiment, PDCE10 is 0.200 to 0.600, 0.300 to 0.500, about 0.400 or is 0.400.

In an embodiment, PDCE11 is 0.125 to 0.525, 0.225 to 0.425, about 0.325 or is 0.325.

In an embodiment, PDCE12 is 0.100 to 0.500, 0.200 to 0.400, about 0.300 or is 0.300.

In an embodiment, PDCE13 is −0.202 to −0.602, −0.302 to −0.502, about −0.402 or is −0.402.

In an embodiment, PDCE14 is 0.673 to 1.073, 0.773 to 0.973, about 0.873 or is 0.873.

In an embodiment, if the subject has one or more first-degree relatives who have, or who have had, colorectal cancer they are given a score of 1, and if the subject does not have one or more first-degree relatives who have, or who have had, colorectal cancer they are given a score of 0.

In an embodiment, if the subject has ever smoked they are given a score of 1, and if the subject has not ever smoked they are given a score of 0.

In an embodiment, if the subject has had a colorectal screen in the last 10 years they are given a score of 1, and if the subject has not had a colorectal screen in the last 10 years they are given a score of 0.

With respect to the two above equations, 3.296 in relation to bmi or trigly is used to centre the variables around zero. In some embodiments, this value is 2.296 to 4.296, 2.756 to 3.796, about 3.296 or is 3.296.

In an embodiment, the clinical and genetic relative risk assessments are combined by determining:

$$crc\_rr = prs\_rr \times fh\_rr .$$

The subject's results can be one or more or all of their absolute 5-year risk, absolute 10-year risk, absolute remaining lifetime risk up to age 90 years and absolute full-lifetime risk to age 90 years, which can be calculated as defined below. In an embodiment, the subject's results are their absolute 10-year risk.

For each individual (aged b years), use the most recent sex-specific, country-specific and ethnicity-specific (if available) population incidence data to determine the population incidence of colorectal cancer from birth to age b years (incid_b), to age b+10 years (incid_b_10) and full lifetime from birth to age 90 years (incid_full_life). Provided below are the calculations for 10-year risks, but any other period can be chosen (e.g. 5-year risks, 15-year risks etc.).

Population incidence rates are available from, for example, the Australian Institute of Health and Welfare, the Surveillance, Epidemiology and End Results program (US), the Office for National Statistics (UK), or any other country's national cancer statistics clearinghouse.

For instance, for Model 1 in Example 1:

### Cumulative Risks

$$cumul\_b = 1 - {e}^{- crc \_ rr \times incid \_ b}$$

$$cumul\_b \_ 10 = 1 - {e}^{- crc \_ rr \times incid \_ b \_ 10}$$

$$cumul\_full \_life = 1 - {e}^{- crc \_ rr \times incid \_ full \_ life}$$

### Absolute 10-Year Risk

$$crc\_risk \_ 10 yr = \frac{( cumul\_b \_ 10 - cumul\_b )}{( 1 - cumul\_b )}$$

### Absolute Remaining Lifetime Risk (to Age 90 Years)

$$crc\_risk \_rem \_life = \frac{( cumul\_full \_life - cumul\_b )}{( 1 - cumul\_b )}$$

### Absolute Full-Lifetime Risk (to Age 90 Years)

$$crc\_risk \_life = cumul\_full \_life$$

As the skilled person would appreciate, for Models 2 and 3 in Example 2, crc_rr in the above equations is replaced with RRmulti_w, RRmulti_m, RRfhprs_w or RRfhprs_m where relevant.

In an embodiment, one or more threshold value(s) are set for determining a particular action such as the need for routine diagnostic testing/screening, preventative therapy or preventative surgery. For example, a score determined using a method of the invention is compared to a pre-determined threshold, and if the score is higher than the threshold a recommendation is made to take the pre-determined action. Methods of setting such thresholds have now become widely used in the art and are described in, for example, US20140018258.

### Subjects

The term “subject” as used herein refers to a human subject. Terms such as “subject”, “patient” or “individual” are terms that can, in context, be used interchangeably in the present disclosure. In an example, the methods of the present disclosure can be used for routine screening of subjects. Routine screening can include testing subjects at pre-determined time intervals. Exemplary time intervals include screening monthly, quarterly, six monthly, yearly, every two years or every three years.

Current risk data suggests that the average person meets the risk threshold for fecal occult blood test screening (which most national screening programs recommend) at around 50 years of age. However, the present inventors have found using the methods of the present disclosure that some individuals should be subject to fecal occult blood test screening well before they reach 50 years of age, in particular if a first degree relative of these subjects has been diagnosed with colorectal cancer. These findings suggest that subjects less than 50 years of age should be assessed using the methods of the present disclosure. Accordingly, in an example, subjects screened using the methods of the present disclosure are at least 38, at least 39, at least 40, at least 41, at least 42, at least 43, at least 44, at least 45, at least 46, at least 47, at least 48, at least 49 years of age. In an example, the subject is at least 40 years of age.

Subjects who have a family history of colorectal cancer can be screened earlier. For example, these subjects can be screened from at least 30, at least 31, at least 32, at least 33, at least 34, at least 35, at least 36, at least 37 years of age or older.

The methods of the present disclosure can be used to assess risk in male and female subjects. However, in an example, the subject is male.

In an embodiment, the methods of the present disclosure can be used for assessing the risk for developing colorectal cancer in human subjects from various ethnic backgrounds. For example, the subject can be classified as Caucasoid, Australoid, Mongoloid and Negroid based on physical anthropology. In particular, the inventors have found that the model can be used for Caucasians, African ancestry (including African Americans), East Asian ancestry and Hispanic ancestry.

In an embodiment, the subject is Caucasian.

In an embodiment, the subject is African.

In an embodiment, the subject is East Asian. In an embodiment, the East Asian subject is from China, Japan, South Korea, North Korea, Taiwan, Hong Kong, Mongolia or Macao.

In an embodiment, the subject is South Asian. In an embodiment, the South Asian subject is from India, Pakistan, Bangladesh, Nepal, Bhutan or Sri Lanka.

In an embodiment, the subject is Hispanic and/or Latino.

It is well known that over time there has been blending of different ethnic origins. However, in practice this does not influence the ability of a skilled person to practice the invention.

A subject of predominantly European origin, either direct or indirect through ancestry, with white skin is considered Caucasian in the context of the present disclosure. A Caucasian may have, for example, at least 75% Caucasian ancestry (for example, but not limited to, the subject having at least three Caucasian grandparents).

A subject of predominantly central or southern African origin, either direct or indirect through ancestry, is considered Negroid in the context of the present disclosure. A Negroid may have, for example, at least 75% Negroid ancestry. An American subject with predominantly Negroid ancestry and black skin is considered African American in the context of the present disclosure. An African American may have, for example, at least 75% Negroid ancestry. A similar principle applies to, for example, subjects of Negroid ancestry living in other countries (for example Great Britain, Canada and The Netherlands).

A subject predominantly originating from Spain or a Spanish-speaking country, such as a country of northern, central or southern America, either direct or indirect through ancestry, is considered Hispanic in the context of the present disclosure. A Hispanic may have, for example, at least 75% Hispanic ancestry.

The terms “ethnicity” and “race” can be used interchangeably in the context of the present disclosure. In an embodiment, the genetic risk assessment can readily be practiced based on what ethnicity the subject considers them self to be. Thus, in an embodiment, the ethnicity of the human subject is self-reported by the subject. As an example, the subject can be asked to identify their ethnicity in response to this question: “To what ethnic group do you belong?” In another example, the ethnicity of the subject is derived from medical records after obtaining the appropriate informed consent from the subject or from the opinion or observations of a clinician.

### Risk Reduction

A high propensity for colorectal cancer can be treated as a warning to commence prophylactic treatment, increase screening frequency or modify screening methods. Thus, after performing the methods of the present disclosure treatment may be prescribed or administered to the subject. In an embodiment, the methods of the present disclosure relate to an anti-colorectal cancer therapy for use in preventing or reducing the risk of colorectal cancer in a human subject at risk thereof. In this embodiment, the subject may be prescribed or administered a therapeutic or prophylactic agent. For example, the subject may be prescribed or administered a chemopreventative. In other examples, the subject may be prescribed or administered nonsteroidal anti-inflammatory drug(s) such as aspirin, ibuprofen, acetaminophen, and naproxen or hormone therapy (estrogen plus progestin). In another example, treatment may include behavioural intervention such as manipulation of the subject's diet. Exemplary dietary modifications include increased fibre, mono-saturated fatty acids and/or fish oil. In another example, risk-reducing screening may include colonoscopy where polypectomy may happen concurrently. In another example, flexible sigmoidoscopy may be offered. In another example, colonoscopy may be offered. In another example, non-invasive screening may include fecal occult blood tests and/or fecal immunochemical testing.

### Sample Preparation and Analysis

In performing the methods of the present disclosure, a biological sample from a subject is required. It is considered that terms such as “sample” and “specimen” are terms that can, in context, be used interchangeably in the present disclosure. Any biological material can be used as the above-mentioned sample so long as it can be derived from the subject and DNA can be isolated and analyzed according to the methods of the present disclosure. Samples are typically taken, following informed consent, from a patient by standard medical laboratory methods. The sample may be in a form taken directly from the patient, or may be at least partially processed (purified) to remove at least some non-nucleic acid material.

Exemplary “biological samples” include bodily fluids (blood, saliva, urine etc.), biopsy, tissue, and/or waste from the patient. Thus, tissue biopsies, stool, sputum, saliva, blood, lymph, tears, sweat, urine, vaginal secretions, or the like can easily be screened for SNPs, as can essentially any tissue of interest that contains the appropriate nucleic acids. In one embodiment, the biological sample is a cheek cell sample.

In another embodiment the sample is a blood sample. A blood sample can be treated to remove particular cells using various methods such as such centrifugation, affinity chromatography (e.g. immunoabsorbent means), immunoselection and filtration if required. Thus, in an example, the sample can comprise a specific cell type or mixture of cell types isolated directly from the subject or purified from a sample obtained from the subject. In an example, the biological sample is peripheral blood mononuclear cells (pBMC). Various methods of purifying sub-populations of cells are known in the art. For example, pBMC can be purified from whole blood using various known Ficoll based centrifugation methods (e.g. Ficoll-Hypaque density gradient centrifugation).

DNA can be extracted from the sample for detecting SNPs. In an example, the DNA is genomic DNA. Various methods of isolating DNA, in particular genomic DNA are known to those of skill in the art. In general, known methods involve disruption and lysis of the starting material followed by the removal of proteins and other contaminants and finally recovery of the DNA. For example, techniques involving alcohol precipitation; organic phenol/chloroform extraction and salting out have been used for many years to extract and isolate DNA. There are various commercially available kits for genomic DNA extraction (Qiagen, Life technologies; Sigma). Purity and concentration of DNA can be assessed by various methods, for example, spectrophotometry.

### Marker Detection Strategies

Amplification primers for amplifying markers (e.g., marker loci) and suitable probes to detect such markers or to genotype a sample with respect to multiple marker alleles, can be used in the disclosure. For example, primer selection for long-range PCR is described in U.S. Ser. Nos. 10/042,406 and 10/236,480; for short-range PCR, U.S. Ser. No. 10/341,832 provides guidance with respect to primer selection. Also, there are publicly available programs such as Oligo available for primer design. With such available primer selection and design software, the publicly available human genome sequence and the polymorphism locations, one of skill can construct primers to amplify the polymorphisms to practice the disclosure. Further, it will be appreciated that the precise probe to be used for detection of a nucleic acid comprising a polymorphism (e.g., an amplicon comprising the polymorphism) can vary, e.g., any probe that can identify the region of a marker amplicon to be detected can be used in conjunction with the present disclosure. Further, the configuration of the detection probes can, of course, vary. Thus, the disclosure is not limited to the sequences recited herein.

Indeed, it will be appreciated that amplification is not a requirement for marker detection; for example, one can directly detect unamplified genomic DNA simply by performing a Southern blot on a sample of genomic DNA.

Typically, molecular markers are detected by any established method available in the art, including, without limitation, ASH, detection of extension, array hybridization (optionally including ASH), or other methods for detecting polymorphisms, AFLP detection, amplified variable sequence detection, randomly amplified polymorphic DNA (RAPD) detection, RFLP detection, self-sustained sequence replication detection, SSR detection, and single-strand conformation polymorphisms (SSCP) detection.

As the skilled person will appreciate, the sequence of the genomic region to which these oligonucleotides hybridize can be used to design primers which are longer at the 5′ and/or 3′ end, possibly shorter at the 5′ and/or 3′ (as long as the truncated version can still be used for amplification), which have one or a few nucleotide differences (but nonetheless can still be used for amplification), or which share no sequence similarity with those provided but which are designed based on genomic sequences close to where the specifically provided oligonucleotides hybridize and which can still be used for amplification.

In some embodiments, the primers are radiolabelled, or labelled by any suitable means (e.g., using a non-radioactive fluorescent tag), to allow for rapid visualization of differently sized amplicons following an amplification reaction without any additional labelling step or visualization step. In some embodiments, the primers are not labelled, and the amplicons are visualized following their size resolution, e.g., following agarose or acrylamide gel electrophoresis. In some embodiments, ethidium bromide staining of the PCR amplicons following size resolution allows visualization of the different size amplicons.

It is not intended that the primers be limited to generating an amplicon of any particular size. For example, the primers used to amplify the marker loci and alleles herein are not limited to amplifying the entire region of the relevant locus, or any subregion thereof. The primers can generate an amplicon of any suitable length for detection. In some embodiments, marker amplification produces an amplicon at least 20 nucleotides in length, or alternatively, at least 50 nucleotides in length, or alternatively, at least 100 nucleotides in length, or alternatively, at least 200 nucleotides in length. Amplicons of any size can be detected using the various technologies described herein. Differences in base composition or size can be detected by conventional methods such as electrophoresis.

Some techniques for detecting genetic markers utilize hybridization of a probe nucleic acid to nucleic acids corresponding to the genetic marker (e.g., amplified nucleic acids produced using genomic DNA as a template). Hybridization formats, including, but not limited to: solution phase, solid phase, mixed phase, or in situ hybridization assays are useful for allele detection. An extensive guide to the hybridization of nucleic acids is found in Tijssen (1993) Laboratory Techniques in Biochemistry and Molecular Biology-Hybridization with Nucleic Acid Probes, Elsevier, New York, as well as in Sambrook et al. (supra).

PCR detection using dual-labelled fluorogenic oligonucleotide probes, commonly referred to as TaqMan™ probes, can also be performed according to the present disclosure. These probes are composed of short (e.g., 20-25 base) oligodeoxynucleotides that are labelled with two different fluorescent dyes. On the 5′ terminus of each probe is a reporter dye, and on the 3′ terminus of each probe a quenching dye is found. The oligonucleotide probe sequence is complementary to an internal target sequence present in a PCR amplicon. When the probe is intact, energy transfer occurs between the two fluorophores and emission from the reporter is quenched by the quencher by FRET. During the extension phase of PCR, the probe is cleaved by 5′ nuclease activity of the polymerase used in the reaction, thereby releasing the reporter from the oligonucleotide-quencher and producing an increase in reporter emission intensity. Accordingly, TaqMan™ probes are oligonucleotides that have a label and a quencher, where the label is released during amplification by the exonuclease action of the polymerase used in amplification. This provides a real time measure of amplification during synthesis. A variety of TaqMan™ reagents are commercially available, e.g., from Applied Biosystems (Division Headquarters in Foster City, Calif.) as well as from a variety of specialty vendors such as Biosearch Technologies (e.g., black hole quencher probes). Further details regarding dual-label probe strategies can be found, e.g., in WO 92/02638.

Other similar methods include e.g. fluorescence resonance energy transfer between two adjacently hybridized probes, e.g., using the LightCycler® format described in U.S. Pat. No. 6,174,670.

Array-based detection can be performed using commercially available arrays, e.g., from Affymetrix (Santa Clara, Calif.) or other manufacturers. Array based detection is one preferred method for identification markers of the disclosure in samples, due to the inherently high-throughput nature of array based detection.

The nucleic acid sample to be analysed is isolated, amplified and, typically, labelled with biotin and/or a fluorescent reporter group. The labelled nucleic acid sample is then incubated with the array using a fluidics station and hybridization oven. The array can be washed and or stained or counter-stained, as appropriate to the detection method. After hybridization, washing and staining, the array is inserted into a scanner, where patterns of hybridization are detected. The hybridization data are collected as light emitted from the fluorescent reporter groups already incorporated into the labelled nucleic acid, which is now bound to the probe array. Probes that most clearly match the labelled nucleic acid produce stronger signals than those that have mismatches. Since the sequence and position of each probe on the array are known, by complementarity, the identity of the nucleic acid sample applied to the probe array can be identified.

Examples of probes which can be used for the invention include, but are not limited to, those provided as SEQ ID NOs 1 to 140.

Thus, in another embodiment, the present disclosure provides a genetic array comprising at least one probe comprising a sequence of nucleotides selected from those provided as SEQ ID NOs 1 to 140. In an embodiment, the array comprises at least 50, at least 100, at least 120 or each of the probes.

Markers and polymorphisms can also be detected using DNA sequencing. DNA sequencing methods are well known in the art and can be found for example in Ausubel et al, eds., Short Protocols in Molecular Biology, 3rd ed., Wiley, (1995) and Sambrook et al, Molecular Cloning, 2nd ed., Chap. 13, Cold Spring Harbor Laboratory Press, (1989). Sequencing can be carried out by any suitable method, for example, dideoxy sequencing, chemical sequencing, or variations thereof.

Suitable sequencing methods also include Second Generation, Third Generation, or Fourth Generation sequencing technologies, all referred to herein as “next generation sequencing”, including, but not limited to, pyrosequencing, sequencing-by-ligation, single molecule sequencing, sequence-by-synthesis (SBS), massive parallel clonal, massive parallel single molecule SBS, massive parallel single molecule real-time, massive parallel single molecule real-time nanopore technology, etc. A review of some such technologies can be found in (Morozova and Marra, 2008), herein incorporated by reference. Accordingly, in some embodiments, performing a genetic risk assessment as described herein involves detecting the at least two polymorphisms by DNA sequencing. In an embodiment, the at least two polymorphisms are detected by next generation sequencing.

Next generation sequencing (NGS) methods share the common feature of massively parallel, high-throughput strategies, with the goal of lower costs in comparison to older sequencing methods (see, for example, Voelkerding et al., 2009; MacLean et al., 2009).

### Computer-Implemented Method

It is envisaged that the methods of the present disclosure may be implemented by a system such as a computer-implemented method. For example, the system may be a computer system comprising one or a plurality of processors which may operate together (referred to for convenience as “processor”) connected to a memory. The memory may be a non-transitory computer-readable medium, such as a hard drive, a solid-state disk, CD-ROM or the cloud. Software, that is executable instructions or program code, such as program code grouped into code modules, may be stored on the memory, and may, when executed by the processor, cause the computer system to perform functions such as determining that a task is to be performed to assist a user to determine the risk of a human subject for developing melanoma; receiving data relating to one or more clinical factors as discussed herein, receiving data relating to the genetic risk assessment, wherein the genetic risk was derived by detecting at least two polymorphisms known to be associated with melanoma; processing the data to obtain the risk of a human subject for developing melanoma; outputting the risk of a human subject for developing melanoma.

For example, the memory may comprise program code, which when executed by the processor causes the system to determine at least two polymorphisms known to be associated with melanoma; process the data to combine clinical and genetic risk assessments to obtain the risk of a human subject for developing melanoma; report the risk of a human subject for developing melanoma.

In another embodiment, the system may be coupled to a user interface to enable the system to receive information from a user and/or to output or display information. For example, the user interface may comprise a graphical user interface, a voice user interface or a touchscreen.

In an embodiment, the system may be configured to communicate with at least one remote device or server across a communications network such as a wireless communications network. For example, the system may be configured to receive information from the device or server across the communications network and to transmit information to the same or a different device or server across the communications network. In other embodiments, the system may be isolated from direct user interaction.

In another embodiment, the diagnostic or prognostic rule is based on the application of a statistical and machine learning algorithm. Such an algorithm uses relationships between a population of polymorphisms and disease status observed in training data (with known disease status) to infer relationships which are then used to determine the risk of a human subject for developing melanoma in subjects with an unknown risk. An algorithm is employed which provides a risk of a human subject developing melanoma. The algorithm performs a multivariate or univariate analysis function.

### EXAMPLES

### Example 1—Materials and Methods—Model 1

### Study Sample

The UK Biobank comprises 500,000 volunteers aged 40-69 years, who were recruited between 2006-2010 from England, Scotland and Wales. The UK Biobank's aim is to enable researchers to study determinants of various diseases, disease prevention and diagnosis and treatment (Sudlow et al., 2015; Bycroft et al., 2018). The UK Biobank has Research Tissue Bank approval (REC #16/NW/0274) that covers analysis of data by approved researchers. All participants provided written informed consent to the UK Biobank before data collection began. This research has been conducted using the UK Biobank resource under Application Number 47401.

Each participant has provided detailed personal and medical history information and has undergone physical and biological measurements. Samples provided include blood, urine and saliva. All participants who provided blood have been genotyped and genome-wide SNP data is available for each (Bycroft et al., 2018). All participants have agreed to their health status being followed-up via linkage to health registries and general practice and hospital records. Therefore, the UK Biobank is a powerful resource to study genetic associations and disease risk due to being a prospective cohort, its large size, and the wealth of genetic and clinical information it has and will collect. The eligibility criteria for this study are described in Table 3.

Characteristics of participants and the mean and median PRS (relative risk) for the 45 SNPs, and 10-year and full-lifetime risks for the model incorporating 45 SNPs and family history were previously described in Gafni et al. (2021). The mean age at baseline for the colorectal cancer cases and controls was 61.45 years (standard deviation 6.33) and 57.28 years (standard deviation 7.96), respectively.

TABLE 3
Eligibility criteria.

| N eligible | Criteria | N dropped |
|---|---|---|
| 502,488 | Active participants in UK Biobank | |
| 487,869 | Reported sex same as genetically | 14,619 |
| | determined sex | |
| 409,289 | White British and genetically Caucasian | 78,580 |
| 406,745 | No previous diagnosis of colorectal | 2,544 |
| | cancer at baseline | |
| 404,715 | Aged 40-69 years at assessment date | 2,030 |
| 403,998 | Genome-wide SNP data available | 717 |

### Generation of PRS

A PRS was calculated for each UK Biobank participant using 140 SNPs (Table 1) associated with colorectal cancer by previous studies (Thomas et al., 2020). Using the method of Mealiffe et al. (2010), the inventors computed for each SNP a (relative) risk score utilising previously published estimates of the odds ratio (OR) per effect allele and effect allele frequency (p) each individual SNP, the inventors calculated the unscaled population average risk using the formula:

$$μ = {( 1 - p )}^{2} + 2 p ( 1 - p ) OR + {p}^{2} {OR}^{2}$$

Weighted risk values were used to normalise the population average to 1, which were calculated as 1/μ, OR/μ and OR2/μ for the three genotypes (defined by the number of effect alleles 0, 1, or 2). The polygenic risk score for each participant was generated by multiplying the weighted risk values for each of the 45 SNPs (assuming independent and additive risks on the log odds scale).

### Outcome

The outcome of interest was invasive colorectal cancer diagnosis after baseline assessment. Colorectal cancer was identified using linked cancer registry data using ICD-9 (1530-1539, 1540-1541), ICD-10 (C18-C20) codes or self-reported disease. Follow-up began at date of baseline assessment and observations were censored at the earliest of date of diagnosis, date of death or 31 Mar. 2016 (the latest date for which linkage to cancer registries is complete), whichever occurred first.

### Risk Scores

Relative risks for the family history model were obtained from Roos et al. (2019). The risk prediction model was generated by multiplying the family history and the 140-SNP PRS relative risks. Data from the UK Office for National Statistics (ONS, 2013) was used to calculate absolute 10-year and full-lifetime risk. SIRs were calculated using the observed versus expected colorectal cancer incidence based on population-based gender- and age-specific incidence rates for England in 2006-2016 (ONS, 2006-2016). Confidence intervals for the SIRs were calculated using the default method of a quadratic approximation to the Poisson log likelihood for the log-rate parameter (StataCorp, 2019).

### Statistical Analysis

Model discrimination was determined using the area under the receiver operating characteristic curve (AUC). The inventors assessed model calibration using logistic regression analysis (MacInnes et al., 2013), for which the observed colorectal cancer case status was the dependent variable and the log-odds of our model's predicted probability for the outcome of colorectal cancer during the follow-up time was the independent variable. The test for dispersion was performed by evaluating the null hypothesis that the estimated regression coefficient was equal to 1 in the model without a constant term (MacInnes et al., 2013). Overdispersion occurs when the observed values have greater variability than the expected values produced by the model, while under-dispersion occurs when the observed values show less variation than expected. This is measured using logistic regression where a slope >1 suggests predicted risks are too extreme and a slope <1 suggests predicted risks are too moderate. The inventors used logistic regression with no intercept terms to assess dispersion for the 10-year risk and full lifetime risk for the combined model.

Broad sense calibration was measured using 10-year follow-up data from the UK Biobank, for which the SIR (observed/expected incidence) was calculated for both models.

All statistical analyses were performed using Stata version 16.1 (2019). All statistical tests were two sided and p<0.05 was considered nominally statistically significant.

### Example 2—Results—Model 1

### Risk Stratification

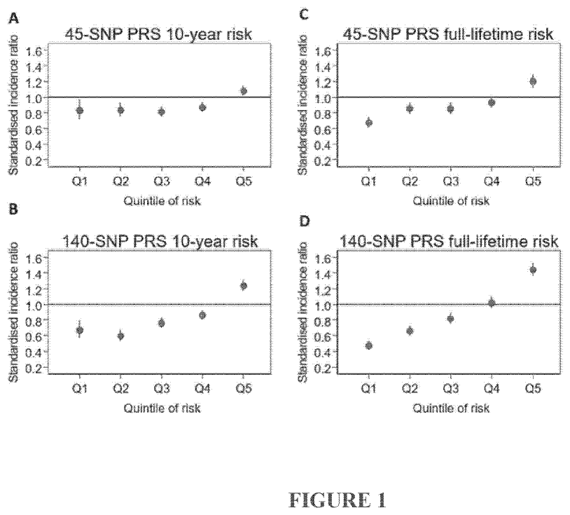

Table 4 shows a comparison between quintiles of SIR for the model using the 140-SNP PRS. The inventors found that using the 140-SNP PRS resulted in improved stratification of risk, as shown by the lower SIR in the bottom quintile of risk and higher SIR in the top quintile of risk. These results are illustrated in FIG. 1 where SIR per quintile of risk is plotted. Compared to the model with the 45-SNP PRS, the model with the 140-SNP PRS shows dramatic improvement in risk stratification between the bottom and the top quintiles of risk, for both 10-year (FIG. 1A vs FIG. 1C) and full-lifetime risk (FIG. 1B vs FIG. 1D).

TABLE 4
Standardised incidence ratios (SIR) by quintile of risk
for the combined 140-SNP PRS and family history model.

| | | | | |
|---|---|---|---|---|
| N | O | E | SIR | 95% Cl |

140-SNP PRS and family history - 10-year risk

| | | | | | |
|---|---|---|---|---|---|
| Quintile 1 (lowest) | 80,336 | 134 | 199.39 | 0.67 | 0.57-0.80 |
| Quintile 2 | 80,464 | 263 | 439.82 | 0.60 | 0.53-0.67 |
| Quintile 3 | 80,706 | 505 | 662.65 | 0.76 | 0.70-0.83 |
| Quintile 4 | 80,942 | 741 | 861.15 | 0.86 | 0.80-0.92 |
| Quintile 5 (highest) | 81,550 | 1349 | 1090.18 | 1.24 | 1.17-1.30 |

140-SNP PRS and family history - full lifetime risk

| | | | | | |
|---|---|---|---|---|---|
| Quintile 1 (lowest) | 80,461 | 259 | 547.44 | 0.47 | 0.42-0.53 |
| Quintile 2 | 80,598 | 397 | 603.41 | 0.66 | 0.57-0.73 |
| Quintile 3 | 80,733 | 532 | 647.84 | 0.82 | 0.75-0.89 |
| Quintile 4 | 80,911 | 710 | 695.88 | 1.02 | 0.95-1.10 |
| Quintile 5 (highest) | 81,295 | 1094 | 758.62 | 1.44 | 1.36-1.53 |

Note:
The SIR was calculated based on number of cases observed and expected using sex-specific UK population rates of colorectal cancer incidence rates, stratified by full lifetime and 10-year risk categories for the combined model using 45 SNPs versus the combined model using 140 SNPs.
Abbreviations: O = observed, E = expected, SIR = standardised incidence ratio, CI = confidence interval.

### Model Performance

For full-lifetime risk, the AUC for the model using the 140-SNP PRS was 0.706 (95% CI 0.697-0.715) while the AUC for the previous model using the 45-SNP PRS was 0.673 (95% CI 0.664-0.682. For 10-year risk, the AUC of the model using the 140-SNP PRS was 0.706 (95% CI 0.698-0.715) while the AUC for the previous model using the 45-SNP PRS was 0.674 (95% CI 0.665-0.683). The 140-SNP PRS model had substantially improved discrimination compared with the 45-SNP PRS model for both 10-year risk (χ²=118.13, df=1, p<0.001) and full-lifetime risk (χ²=122.13, df=1, p<0.001).

In terms of the calibration, the 10-year risk for the 140-SNP PRS model was marginally under-dispersed (dispersion coefficient 1.10, 95% CI 1.09-1.11), whereas the full lifetime risk was substantially under-dispersed (dispersion coefficient 1.87, 95% CI 1.85-1.18). The inventors assessed broad sense calibration by analysing the SIR of the observed number of cases compared with model predictions. A small overestimation of risk in the model with the 140-SNP PRS (SIR=0.951, 95% CI 0.918-0.986) was found.

### The Model-1

In an embodiment, the clinical risk assessment if the subject has no first-degree relatives who have, or who have had, colorectal cancer (fh_rr) is 0.92, or if the subject has one or more first-degree relatives who have, or who have had, colorectal cancer (fh_rr) is 2.1.

For each single-nucleotide polymorphism (SNP) in the panel of 140 SNPs in Table 1, the unscaled population average risk is calculated using the method of Mealiffe et al (2010) as:

$$μ = {( 1 - p )}^{2} + 2 p ( 1 - p ) OR + {p}^{2} {OR}^{2} ,$$

where OR is the odds ratio per effect allele and p is the effect allele frequency.

For each individual, the adjusted risk (which has a population mean equal to 1) for each of the SNPs is calculated as:

$$\frac{{OR}^{N}}{μ} ,$$

where N is the individual's number of effect alleles for the SNP.

Any individual who is missing genotype data for one or more SNPs is given an adjusted risk of 1 for each missing SNP.

The polygenic relative risk score (prs_rr) for each participant is the product of their adjusted risks for the SNPs.

The combined relative risk is calculated as: crc_rr=prs_rr×fh_rr.

For each individual (aged b years), use the most recent sex-specific, country-specific and ethnicity-specific (if available) population incidence data to determine the population incidence of colorectal cancer from birth to age b years (incid_b), to age b+10 years (incid_b_10) and full lifetime from birth to age 90 years (incid_full_life). Provided below are the calculations for 10-year risks, but any other period can be chosen (e.g. 5-year risks, 15-year risks etc.).

Population incidence rates are available from, for example, the Australian Institute of Health and Welfare, the Surveillance, Epidemiology and End Results program (US), the Office for National Statistics (UK), or any other country's national cancer statistics clearinghouse.

### Cumulative Risks

$$cumul\_b = 1 - {e}^{- crc \_ rr \times incid \_ b}$$

$$cumul\_b \_ 10 = 1 - {e}^{- crc \_ rr \times incid \_ b \_ 10}$$

$$cumul\_full \_life = 1 - {e}^{- crc \_ rr \times incid \_ full \_ life}$$

### Absolute 10-Year Risk

$$crc\_risk \_ 10 yr = \frac{( cumul\_b \_ 10 - cumul\_b )}{( 1 - cumul\_b )}$$

### Absolute Remaining Lifetime Risk (to Age 90 Years)

$$crc\_risk \_rem \_life = \frac{( cumul\_full \_life - cumul\_b )}{( 1 - cumul\_b )}$$

### Absolute Full-Lifetime Risk (to Age 90 Years)

$$crc\_risk \_life = cumul\_full \_life$$

### Example 3—Materials and Methods—Models 2 and 3

### Eligibility

The inventors extracted data for participants who had not withdrawn their consent by 25 Apr. 2023, whose genetic sex was the same as their gender identity and who were aged 40 to 60 years at their baseline assessment. Participants were excluded from this study if they had been diagnosed with colorectal cancer before their baseline assessment date; did not have genotyping data available; had a history of polyps, Chron's disease or ulcerative colitis; or had been diagnosed with colorectal cancer or died within the first six weeks of follow-up. So that the dataset did not have closely related pairs of participants, we used the ukb_gen_samples_to_remove function of the R package ukbtools (Handscombe et al., 2019). This package identifies related pairs (the inventors used a criterion of closer than third-degree relatedness) and randomly chooses one to be removed from the dataset. The last step was to restrict the dataset to participants who had a genetically determined UK ancestry. Table 5 provides details of the eligibility criteria and the number of participants eligible and dropped at each step.

TABLE 5
Eligibility criteria and the number
eligible and dropped at each step.

| N eligible | Criteria | N dropped |
|---|---|---|
| 502,366 | Active UK Biobank participant (on 25 Apr. 2023) | |
| 501,988 | Gender identity same as genetic sex | 378 |
| 498,841 | Aged 40-69 years at baseline assessment date | 3,147 |
| 495,977 | No colorectal cancer at baseline assessment date | 2,864 |
| 480,978 | Genotyping data available | 14,999 |
| 466,459 | No history of polyps, Chron's disease or | 14,519 |
| | ulcerative colitis | |
| 466,426 | Alive after six weeks of follow-up | 33 |
| 466,399 | Unaffected after six weeks of follow-up | 27 |
| 434,476 | Unrelated individuals (≥3rd degree relatedness) | 31,923 |
| 396,072 | Genetically determined UK ancestry | 38,404 |

### Data Extraction

Details of the UK Biobank data fields used to derive variables for analysis and eligibility assessment are in Table 6. For first-degree family history of colorectal cancer, the data fields cover mother, father and any sibling; there is no way of knowing if more than one sibling has been affected. Very few participants (0.5%) had two or more affected first-degree relatives; therefore, the inventors used family history as a binary variable for having any affected first-degree relative. The physical activity fields were combined into a summary measure, using the short format calculation of the metabolic equivalent of task in Craig et al. (2003) and dividing by 1,000. For women whose menopausal status was unknown (because of hysterectomy or another reason), their status was adjudicated using hormone replacement therapy (HRT) use (menopausal if she had ever taken HRT) and age at baseline assessment (for women who had never taken HRT, premenopausal if aged <51 years and menopausal if aged ≥51 years). Menopause and HRT use were combined into a single risk factor with categories for premenopausal, menopausal with no HRT and menopausal with HRT. To identify participants with genetically determined United Kingdom ancestry, the inventors used the ancestry categories that were defined by principal components analysis by Privé et al. (2022) and made available for download from the UK Biobank.

TABLE 6
UK Biobank data fields used to derive variables for analysis and eligibility assessment

| | | Related age or | |
|---|---|---|---|
| Variable | Data fields | date fields | Note |
| Age at baseline assessment | 21003 | 34, 52, 53 | Calculated from baseline assessment date |
| | | | and month and year of birth |
| Genetic sex/gender identity | 22001, 31 | | |
| Colorectal cancer diagnosis | 20001, 40006, | 20006, 20007, | 20001 = 1020, 1022, 1023; 40006 = C18*, |
| | 40013 | 40005, 40008 | C19*, C20*; 40013 = 153*, 1540, 1541 |
| Age at death | 40000 | 40007 | |
| First-degree family history | 20107, 20110, | | Mother, 20110 = 4; father, 20107 = 4, |
| of colorectal cancer | 20111 | | sibling, 20111 = 4; there is no way of |
| | | | knowing if more than one sibling is affected |
| Body mass index | 21001 | | |
| Polyps | 20002, 20004, | 20008, 20010, | 20002 = 1460; 20004 = 1463; |
| | 41270, 41272 | 20011 41280, | 41270 = K621, K635; 41272 = H20*, |
| | | 41282 | H23*, H26* |
| Chron's disease | 131626 | | Before baseline assessment date |
| Ulcerative colitis | 131628 | | Before baseline assessment date |
| Type 2 or unspecified diabetes | 130708, 130714 | | Before baseline assessment date |
| Screening procedure for | 20004, 41272 | 20010, 20011, | 20004 = 1463, 1519; 41272 = H20*, |
| colorectal cancer | | 41280, 41282 | H22*, H23*, H25*, H26*, H28* (before |
| | | | baseline assessment date) |
| High-density lipoprotein | 30760 | | |
| Triglycerides | 30870 | | |
| Low-density lipoprotein | 30780 | | |
| Total cholesterol | 30960 | | |
| Non-steroidal anti- | 6154, 20003 | | 6154 = 1, 2; 20003 = 140861806, 1140861808, |
| inflammatory drug use | | | 1140864860, 1140868226, 1140868282, 1140872040, |
| | | | 1140882108, 1140882190, 1140882192, 1140882268, |
| | | | 1140882392, 1140911760, 1141163138, 1141164044, |
| | | | 1141167844, 1140871310, 1140871374, 1140871388, |
| | | | 1140871394, 1140875540, 1140875616, 1140877962, |
| | | | 1140877964, 1140877966, 1140878030, 1140910496, |
| | | | 1140911086, 1140911748, 1140911750, 1140911762, |
| | | | 1140927152, 1140928656, 1141149110, 1141152166, |
| | | | 1141152168, 1141153082, 1141153134, 1141157412, |
| | | | 1141164254, 1141176278, 1141177836, 1141182814, |
| | | | 1141182868, 1141184226, 1141184546, 1141188652, |
| | | | 1141190952, 1141191742, 1141194296, 1141200576, |
| | | | 1141200748, 1140871462, 1140871472, 1140881612, |
| | | | 1140871168, 1140871174, 1140877892, 1140878034, |
| | | | 1140878036, 1140884488, 1140917394, 1140921828, |
| | | | 1141174424, 1141176878, 1141182674, 1141191028, |
| | | | 1141176662, 1141176668, 1141176670, 1140871542, |
| | | | 1140871546, 1141180140, 1141180148, 1141180150, |
| | | | 1141180152, 1140871336, 1141157452 |
| Calcium supplement | 6179, 6155 | | 6179 = 3; 6155 = 7 |
| Fish oil supplement or eat | 6179, 1329 | | 6179 = 1, 1329 = 3, 4, 5 |
| oily fish | | | |
| Vitamin D supplement | 6155 | | 6155 = 4 |
| Hormone replacement therapy | 2814 | 3536, 3546 | |
| Menopause | 2724 | | For 2724 = 2 or 3, menopausal status was |
| | | | adjudicated using hormone replacement therapy |
| | | | status (menopause = yes if hormone |
| | | | replacement therapy = yes) and age at |
| | | | baseline assessment (premenopausal if |
| | | | aged <51 years and menopausal if aged ≥51 years) |
| Physical activity | 864, 874, 884, | | These fields were used to calculate a summary |
| | 894, 904, 914 | | physical activity measure using the short |
| | | | format calculation of the metabolic equivalent |
| | | | of task in Craig et al¹⁴ and dividing by 1000 |
| Alcohol | 1558 | | |
| Smoking | 20116 | 2879 | |
| Processed meat intake | 1349 | | |
| Beef intake | 1369 | | |
| Pork intake | 1389 | | |
| Cereal intake | 1458 | | |
| White bread intake | 1438, 1448 | | 1448 = 1 |
| Wholemeal/wholegrain bread intake | 1438, 1448 | | 1448 = 3 |
| Cooked vegetable intake | 1289 | | |
| Raw vegetable or salad intake | 1299 | | |
| Fresh fruit intake | 1309 | | |
| Dried fruit intake | 1319 | | |

Note:
*represents a wildcard.

The inventors extracted genotypes for the panel of 140 SNPs used in Examples 1 and 2. However from the UK Biobank's SNP imputation dataset using Plink version 1.9.16 17 In the published list of SNPs, the rsID for 13:34092164_C/T had been mis-identified as rs377429877 and should have been rs9537756.

Overall, 58,124 (14.7%) participants had all 140 SNPs genotyped, 110,695 (28.0%) were missing one SNP and 106,068 (26.8%) were missing two SNPs. Only 18,888 (4.8%) were missing five or more SNPs. The PRS for use in developing the new models was calculated as the linear combination of the published betas (Thomas et al., 2020) multiplied by the number of effect alleles for each SNP and then standardised to have a mean of 0 and a standard deviation of 1.

The raw polygenic risk score (PRS) is calculated as the weighted sum of the effect allele counts for the SNPs in the PRS:

$${\sum }_{j = 1}^{p} {β}_{j} {G}_{ij}$$

where βⱼ is the weight for SNP j, Gᵢⱼ is the count (0, 1, 2) of the effect alleles of SNP j for individual i, and p is the number of SNPs in the PRS. The β weights are given in Table 1.

Any individual who is missing genotype data for one or more SNPs is given an adjusted risk of 0 for each missing SNP.

The PRS was standardised to have a mean of 0 and standard deviation of 1 by subtracting the population mean and dividing by the population standard deviation:

$$standardised PRS = \frac{P R {S}_{raw} - P R {S}_{\overline{x}}}{P R {S}_{sd}} ,$$

where PRS_{raw} is the individual's raw PRS, PRS_{x̅} is the population mean of PRS_{raw}, and PRS_{sd} is the population standard deviation of PRS_{raw}.

In these calculations, PRS_{x̅}=8.018 and PRS_{sd}=0.473 but these numbers will change for different populations.

### Statistical Analysis

For each participant, follow-up began at the date of their baseline assessment and ended at the earliest of their date of diagnosis of colorectal cancer or 31 Jul. 2019 (the date to which linkage to cancer registries was complete). The participants were randomly divided into a 70% training dataset and a 30% testing dataset that were balanced for sex and affected status.

In the training dataset, we used all available follow-up, while in the testing dataset, we limited follow-up to 10 years. For the calculation of standardised incidence ratios (SIR) in the testing dataset, follow-up was censored at age of death for participants who had died before completing 10 years of follow-up. Stata (version 18.0) was used for most of the analyses; R¹³ was used for the variable selection for the new multivariable models for men and women. All statistical tests were two sided and P values<0.05 were considered nominally statistically significant.

### Training

Some of the risk factors considered for inclusion in the models had missing data, most notably physical activity, which was missing for 25.2% unaffected and 27.8% affected women and 17.6% unaffected and 19.4% affected men (Table 7). The lipid profile measures were missing for 4.7-13.4% unaffected and 4.7-12.9% affected women and 4.6-11.9% unaffected and 5.3-12.4% affected men. Other risk factors had missing data for less than 2.0% of the participants. The inventors therefore used multiple imputation in the training dataset.

After a pilot analysis using 11 imputations, the inventors determined the required number of imputations using von Hippel's two-stage approach (von Hippel et al., 2020). This calculation uses the upper limit of the 95% confidence interval for the fraction of missing information as input (rather than the point estimate) to ensure that there is only a 2.5% chance that the required number of imputations will be underestimated. With all variables under consideration included, 30 imputations were required, a number driven by the large proportion of missing information for physical activity and the blood lipid measures (omitting physical activity reduced the number of imputations needed to 7; also omitting the blood lipids reduced the number to two).

Given the lengthy computation time required for each imputation, the inventors took a pragmatic approach and repeated the calculation for all variables using the point estimate of the fraction of missing information (0.25) and used 14 imputations for development of the models. Once the models were developed, repeated the calculation using the upper limit of the 95% confidence interval for the fraction of missing information as the input to ensure that the number of imputations was adequate.

TABLE 7
Summary statistics for unaffected and affected women and men for baseline risk factors
considered in the development of the colorectal cancer risk prediction models

| | Women | | | | Men | | | |
|---|---|---|---|---|---|---|---|---|
| | Risk factor | | | | | | | |
| | Unaffected | | Affected | | Unaffected | | Affected | |
| Continuous | Mean | SD | Mean | SD | Mean | SD | Mean | SD |
| 140-SNP PRS | 8.04 | 0.46 | 8.24 | 0.46 | 8.04 | 0.46 | 8.23 | 0.46 |
| Body mass index (kg/m²) | 27.01 | 5.15 | 27.19 | 5.01 | 27.83 | 4.24 | 28.44 | 4.31 |
| Physical activity | 2562.89 | 2494.75 | 2502.39 | 2398.12 | 2853.20 | 2980.40 | 2746.06 | 2899.21 |
| (MET-minutes per week) | | | | | | | | |
| Time since last screening | 4.46 | 2.94 | 4.65 | 3.04 | 4.38 | 2.93 | 4.85 | 2.87 |
| procedure, if screened in | | | | | | | | |
| last 10 years (years) | | | | | | | | |
| Cholesterol (mmol/L) | 5.90 | 1.12 | 6.07 | 1.16 | 5.51 | 1.12 | 5.40 | 1.17 |
| High-density lipoprotein | 1.60 | 0.38 | 1.60 | 0.38 | 1.28 | 0.31 | 1.29 | 0.33 |
| (mmol/L) | | | | | | | | |
| Low-density lipoprotein | 3.64 | 0.87 | 3.76 | 0.89 | 3.49 | 0.86 | 3.40 | 0.89 |
| (mmol/L) | | | | | | | | |
| Triglycerides (mmol/L) | 1.55 | 0.85 | 1.70 | 0.90 | 1.98 | 1.15 | 2.00 | 1.13 |
| Cooked vegetables | 2.65 | 1.47 | 2.70 | 1.45 | 2.65 | 1.64 | 2.73 | 1.63 |
| (serves per day) | | | | | | | | |
| Salad or raw vegetables | 2.28 | 1.81 | 2.28 | 1.78 | 1.83 | 1.73 | 1.79 | 1.67 |
| (serves per day) | | | | | | | | |
| Fresh fruit (pieces per day) | 2.35 | 1.46 | 2.40 | 1.44 | 1.98 | 1.48 | 1.98 | 1.52 |

| Categorical | N | % | N | % | N | % | N | % |
|---|---|---|---|---|---|---|---|---|
| Affected first-degree | | | | | | | | |
| relative, any | | | | | | | | |
| No | 188,683 | 88.9 | 1,619 | 84.6 | 157,154 | 87.7 | 2,140 | 82.4 |
| Yes | 22,616 | 10.7 | 280 | 14.6 | 19,843 | 11.1 | 415 | 16.0 |
| Unknown* | 971 | 0.5 | 14 | 0.7 | 2,294 | 1.3 | 43 | 1.7 |
| Screening procedure | | | | | | | | |
| in last 10 years | | | | | | | | |
| No | 195,012 | 91.9 | 1,806 | 94.4 | 167,627 | 93.5 | 2,474 | 95.2 |
| Yes | 17,258 | 8.1 | 107 | 5.6 | 11,664 | 6.5 | 124 | 4.8 |
| Diabetes, type 2 | | | | | | | | |
| or unspecified | | | | | | | | |
| No | 205,639 | 96.9 | 1,833 | 95.8 | 167,956 | 93.7 | 2,348 | 90.4 |
| Yes | 6,631 | 3.1 | 80 | 4.2 | 11,335 | 6.3 | 250 | 9.6 |
| NSAID, regular use | | | | | | | | |
| No | 148,835 | 70.1 | 1,363 | 71.3 | 121,059 | 67.5 | 1,664 | 64.1 |
| Yes | 61,541 | 29.0 | 525 | 27.4 | 56,215 | 31.4 | 890 | 34.3 |
| Unknown | 1,894 | 0.9 | 25 | 1.3 | 2,017 | 1.1 | 44 | 1.7 |
| Menopause and HRT | | | | | | | | |
| Premenopausal | 52,229 | 24.6 | 196 | 10.3 | | | | |
| Menopausal, no HRT | 76,941 | 36.3 | 810 | 42.3 | | | | |
| Menopausal, took HRT | 82,548 | 38.9 | 896 | 46.8 | | | | |
| Missing | 552 | 0.3 | 11 | 0.6 | | | | |
| Calcium supplement | | | | | | | | |
| No | 148,687 | 70.1 | 1,318 | 68.9 | 144,255 | 80.5 | 2,110 | 81.2 |
| Yes | 63,073 | 29.7 | 585 | 30.6 | 34,445 | 19.2 | 482 | 18.6 |
| Unknown | 510 | 0.2 | 10 | 0.5 | 591 | 0.3 | 3 | 0.2 |
| Vitamin D supplement | | | | | | | | |
| No | 153,065 | 72.1 | 1,354 | 70.8 | 142,890 | 79.7 | 2,091 | 80.5 |
| Yes | 58,364 | 27.5 | 547 | 28.6 | 35,144 | 19.6 | 480 | 18.5 |
| Unknown | 841 | 0.4 | 12 | 0.6 | 1,257 | 0.7 | 27 | 1.0 |
| Fish oil supplement or | | | | | | | | |
| eat oily fish two or | | | | | | | | |
| more times per week | | | | | | | | |
| No | 142,033 | 66.9 | 1,197 | 62.6 | 124,754 | 69.6 | 1,741 | 67.0 |
| Yes | 69,629 | 32.8 | 705 | 36.9 | 53,984 | 30.1 | 849 | 32.7 |
| Unknown | 608 | 0.3 | 11 | 0.6 | 643 | 0.4 | 8 | 0.3 |
| Alcohol use | | | | | | | | |
| Never or rarely | 73,547 | 34.7 | 689 | 36.0 | 36,328 | 20.3 | 453 | 17.4 |
| One or two times | 56,100 | 26.4 | 468 | 24.5 | 47,011 | 26.2 | 602 | 23.2 |
| per week | | | | | | | | |
| Three of four times | 46,153 | 21.7 | 366 | 19.1 | 48,650 | 27.1 | 711 | 27.4 |
| per week | | | | | | | | |
| Daily or almost daily | 36,221 | 17.1 | 384 | 20.1 | 47,054 | 26.2 | 829 | 31.9 |
| Unknown | 249 | 0.1 | 6 | 0.3 | 248 | 0.1 | 3 | 0.1 |
| Smoking, ever | | | | | | | | |
| No | 125,045 | 58.9 | 1,023 | 53.5 | 87,901 | 49.0 | 1,001 | 38.5 |
| Yes | 86,392 | 40.7 | 881 | 46.1 | 90,666 | 50.6 | 1,588 | 61.1 |
| Unknown | 833 | 0.4 | 9 | 0.5 | 724 | 0.4 | 9 | 0.4 |
| Processed meat | | | | | | | | |
| (serves per week) | | | | | | | | |
| None | 24,621 | 11.6 | 193 | 10.1 | 8,376 | 4.7 | 80 | 3.1 |
| 1 | 80,587 | 38.0 | 739 | 38.6 | 37,135 | 20.7 | 518 | 19.9 |
| 2 | 62,239 | 29.3 | 573 | 30.0 | 54,294 | 30.3 | 803 | 30.9 |
| 3 or more | 44,429 | 20.9 | 404 | 21.1 | 79,109 | 44.1 | 1,192 | 45.9 |
| Unknown | 394 | 0.2 | 4 | 0.2 | 377 | 0.2 | 5 | 0.2 |
| Beef (serves per week) | | | | | | | | |
| None | 26,015 | 12.3 | 211 | 11.0 | 12,028 | 6.7 | 127 | 4.9 |
| 1 | 98,906 | 46.6 | 888 | 46.4 | 81,239 | 45.3 | 1,172 | 45.1 |
| 2 | 64,067 | 30.2 | 587 | 30.7 | 62,786 | 35.2 | 880 | 33.9 |
| 3 or more | 22,479 | 10.6 | 218 | 11.4 | 22,485 | 12.5 | 410 | 15.8 |
| Unknown | 785 | 0.4 | 9 | 0.5 | 753 | 0.4 | 9 | 0.4 |
| Pork (serves per week) | | | | | | | | |
| None | 39,277 | 18.5 | 325 | 17.0 | 20,675 | 11.5 | 267 | 10.3 |
| 1 | 122,982 | 57.9 | 1,104 | 57.7 | 105,111 | 58.6 | 1,458 | 56.1 |
| 2 | 43,753 | 20.6 | 420 | 22.0 | 44,733 | 25.0 | 719 | 27.7 |
| 3 or more | 5,150 | 2.4 | 50 | 2.6 | 7,663 | 4.3 | 137 | 5.3 |
| Unknown | 1,108 | 0.5 | 14 | 0.7 | 1,139 | 0.6 | 17 | 0.7 |
| Dried fruit | | | | | | | | |
| (serves per day) | | | | | | | | |
| None | 119,756 | 56.4 | 1,071 | 56.0 | 122,036 | 68.1 | 1,831 | 70.5 |
| 1 or more | 90,476 | 42.6 | 822 | 43.0 | 55,398 | 30.9 | 733 | 28.2 |
| Unknown | 2,038 | 1.0 | 20 | 1.1 | 1,857 | 1.0 | 34 | 1.3 |
| Cereal (bowls per week) | | | | | | | | |
| None | 33,790 | 15.9 | 320 | 16.7 | 30,595 | 17.1 | 517 | 19.9 |
| 1-3 | 35,018 | 16.5 | 296 | 15.5 | 31,968 | 17.8 | 468 | 18.0 |
| 4-6 | 57,936 | 27.3 | 485 | 25.4 | 47,219 | 26.3 | 629 | 24.2 |
| 7 or more | 84,998 | 40.0 | 808 | 42.2 | 69,000 | 38.5 | 977 | 37.6 |
| Unknown | 528 | 0.3 | 4 | 0.2 | 509 | 0.3 | 7 | 0.3 |
| White bread | | | | | | | | |
| (slices per week) | | | | | | | | |
| None | 170,436 | 80.3 | 1,524 | 79.7 | 121,247 | 67.6 | 1,694 | 65.2 |
| 1-4 | 6,808 | 3.2 | 52 | 2.7 | 3,301 | 1.8 | 42 | 1.6 |
| 5-10 | 16,629 | 7.8 | 141 | 7.4 | 16,884 | 9.4 | 253 | 9.7 |
| 11 or more | 17,313 | 8.2 | 181 | 9.5 | 36,586 | 20.4 | 580 | 22.3 |
| Unknown | 1,084 | 0.5 | 15 | 0.8 | 1,273 | 0.7 | 29 | 1.1 |
| Wholemeal or wholegrain | | | | | | | | |
| bread (slices per week) | | | | | | | | |
| None | 83,003 | 39.1 | 778 | 40.7 | 88,311 | 49.3 | 1,326 | 51.0 |
| 1-4 | 24,257 | 11.4 | 188 | 9.8 | 6,001 | 3.4 | 88 | 3.4 |
| 5-10 | 53,693 | 25.3 | 482 | 25.2 | 27,874 | 15.6 | 415 | 16.0 |
| 11 or more | 50,042 | 23.6 | 447 | 23.4 | 56,168 | 31.3 | 746 | 28.7 |
| Unknown | 1,275 | 0.6 | 18 | 0.9 | 937 | 0.5 | 23 | 0.9 |

Note:
HRT, hormone replacement therapy; MET, metabolic equivalent task; NSAID, non-steroidal anti-inflammatory drug; PRS, polygenic risk score; SD, standard deviation; SNP, single-nucleotide polymorphism.
BMI was missing for 625 (0.3%) unaffected and 8 (0.4%) affected women and for 636 (0.4%) unaffected and 9 (0.3%) affected men; physical activity was missing for 53,582 (25.2%) unaffected and 532 (27.8%) affected women and for 31,500 (17.5%) unaffected and 505 (19.4%) affected men; total cholesterol was missing for 9,999 (4.7%) unaffected and 90 (4.7%) affected women and for 8,192 (4.6%) unaffected and 137 (5.3%) affected men; high-density lipoprotein was missing for 28,497 (13.4%) unaffected and 246 (12.9%) affected women and for 21,407 (11.9%) unaffected and 323 (12.4%) affected men; low-density lipoprotein was missing for 10,838 (5.1%) unaffected and 92 (4.8%) affected women and for 8,561 (4.8%) unaffected and 141 (5.4%) affected men; triglycerides was missing for 10,113 (4.8%) unaffected and 89 (4.7%) affected women and for 8,370 (4.7%) unaffected and 142 (5.5%) affected men; cooked vegetables consumption was missing for 1,675 (0.8%) unaffected and 17 (0.9%) affected women and for 2,650 (1.5%) unaffected and 45 (1.7%) affected men; salad or raw vegetables consumption was missing for 2,016 (0.9%) unaffected and 21 (1.1%) affected women and for 2,861 (1.6%) unaffected and 45 (1.7%) affected men; fresh fruit consumption was missing for 676 (0.3%) unaffected and 5 (0.3%) affected women and for 891 (0.5%) unaffected and 20 (0.8%) affected men; other continuous variables had no missing data.
*Unknown is no response to family history questions for all of mother, father and siblings. A further 1,628 (0.8%) unaffected and 18 (0.9%) affected women and 2,775 (1.5%) unaffected and 49 (1.9%) affected men were missing for mother and father but not for siblings; 839 (0.4%) unaffected and 5 (0.3%) affected women and 1,769 (1.0%) unaffected and 33 (1.3%) affected men were missing for mother and siblings but not for father; 1,417 (0.7%) unaffected and 14 (0.7%) affected women and 1,893 (1.1%) unaffected and 25 (9.6%) affected men were missing for father and siblings but not for mother; 3,591 (1.7%) unaffected and 29 (1.5%) affected women and 4,716 (2.6%) unaffected and 68 (2.6%) affected men were missing mother only; 10,655 (5.0%) affected and 102 (5.3%) affected women and 8,834 (4.9%) affected and 150 (5.8%) affected men were missing father only; 6,098 (2.9%) affected were and 65 (3.4%) unaffected women and 7,503 (4.2%) affected were and 137 (5.3%) unaffected men were missing sibling only.

For the imputations, the inventors used chained equations: linear regression for BMI, physical activity and the four lipid profile measures; logistic regression for first-degree family history, smoking ever, NSAID use, vitamin D supplements, calcium supplements, fish oil supplements/oily fish intake and dried fruit intake; truncated regression (with an allowed range from 0 to 10) for fresh fruit intake, cooked vegetable intake and raw vegetable/salad intake; predictive mean matching (with 3 nearest neighbours) for cereal intake, wholemeal/wholegrain bread intake and white bread intake; conditional multinomial logistic regression for combined menopause and HRT status for women only; and ordered logistic regression for alcohol use, colorectal cancer screening; processed meat intake, beef intake and pork intake.

In the multiple imputation training dataset, the inventors used age as the time axis and fitted Cox proportional hazards models for women and men separately. First unadjusted hazard ratios for each of the risk factors considered for inclusion in the models was obtained. For the new multivariable models, the inventors performed forwards and backwards stepwise model selection separately on each of the 14 imputed datasets, and for women and men separately. To obtain simple models, the inventors used the Bayesian information criterion as the measure of performance because it penalises additional parameters more than the Akaike information criterion. After the stepwise procedures, the inventors selected variables that appeared in at least half of the models. The inventors fitted Cox proportional hazards models for women and men separately using the selected variables and used Wald tests to determine whether the variables would be retained in the final models. As an alternative to the new multivariable models, the inventors also fitted new models for women and men with only first-degree family history and the PRS as covariates.

The inventors tested the proportional hazards assumption of the new models by including each of the variables as a time-varying covariate. Because this test is sensitive to small deviations from the assumption, the inventors assessed any potentially problematic variables using a plot of the scaled Schoenfeld residuals by age in the first imputation dataset. The fit of the models was assessed using a graph of the Nelson-Aalen cumulative hazard function and the Cox-Snell residuals for the first imputation dataset.

To directly compare the strength of the associations for each of the risk factors in the new models (which had been measured on different scales), the inventors used the odds per adjusted standard deviation approach (Hopper, 2015). In all 14 of the imputation datasets, the inventors used each risk factor as the dependent variable and fitted a linear or logistic regression (as appropriate) with the other risk factors as independent variables. The inventors then obtained the residuals from these models and divided these by their standard deviation. These new variables were then included in Cox regression models. The estimates and standard errors from the 14 imputation datasets for each of the new models were combined using Rubin's rules (Rubin, 2004) before calculation of the 95% confidence intervals and P values.

### Testing

The risk factors included in the new multivariable models for women and men had little missing data: under 0.5% for women and under 1.5% for men (except for triglycerides, which was missing for 4.8% of women, and screening procedure in the last 10 years, which was missing for 8.1% of women and 6.6% of men). The inventors therefore replaced these missing values with the reference value for the categorical variables and the mean value for the continuous variables. For the new models the inventors calculated the linear combination of the risk factors and beta coefficients for each participant and centred this value by subtracting the mean. The inventors then obtained the natural exponential of these centred values and used these as the relative risk in the calculation of absolute risk.

For the current family history and PRS model, he inventors multiplied the population-adjusted PRS by 0.92 if the participant had no first-degree family history of colorectal cancer and by 2.10 if they did, as in Gafni et al. (2021). For the family history alone model, the inventors assigned a value of 0.92 if the participant had no first-degree family history of colorectal cancer and 2.10 if they did (to ensure that the population average risk was equal to 1). The inventors then used these values as the relative risk in the calculation of the absolute risks. Population average risks were calculated using the equations below without a relative risk term.

For the calculation of absolute 10-year risks of colorectal cancer in the testing dataset, the inventors used annual, sex-specific, age-specific and age-standardised population incidences for England (Office for National Statistics, 2019a) for the 10-year risks the inventors applied the competing mortality adjustment in equation 5 of Gail et al. (1989) using annual sex- and age-specific non-colorectal cancer mortality rates from England and Wales (Office for National Statistics, 2016 and 2019b). These incidences and mortality rates are annual and constant in 5- or 10-year periods, so, as in equation 6 of Gail et al. (1989) they reduce to the following explicit formulae.

Let λ₁(t) be the relative risk from the previous section multiplied by the population colorectal cancer incidences (above) for a woman aged t years. Let λ₂(t) be the non-colorectal cancer mortality rates (above) for an individual aged t years. Assume that λ₁(t) is a step function of t that is constant for t in all intervals of the form [k, k+1) for an integer k (this holds true for the incidences and rates, in fact they are constant in larger, 5- or 10-year, intervals). This is the same assumption as in Gail et al. (1989) with τⱼ=j+1 and Δⱼ=1, in their terminology. Then, equation 6 of Gail et al. (1989) says that the probability that an individual will develop colorectal cancer in the next 10 years, given that the individual is currently unaffected and aged a years, is the 10-year risk

$${\sum }_{j = a}^{a + 9} \frac{{λ}_{1} ( j )}{{λ}_{1} ( j ) + {λ}_{2} ( j )} \frac{{S}_{1} ( j )}{{S}_{1} ( a )} \frac{{S}_{2} ( j )}{{S}_{2} ( a )} [ 1 - exp ( - {λ}_{1} ( j ) - {λ}_{2} ( j ) ) ] , where$$

$${S}_{1} ( t ) = exp ( - {λ}_{1} ( 0 ) - {λ}_{1} ( 1 ) - {λ}_{1} ( 2 ) - ⋯ - {λ}_{1} ( t - 1 ) )$$

if t≥1 is an integer, and S₂(t) has the same definition except with a subscript of 2 instead of 1. The inventors calculated the full lifetime colorectal cancer risks using the equations above for ages j=0 to j=89.

The inventors first assessed the extent to which the testing dataset represented women and men in the United Kingdom population by estimating the standardised incidence ratio (SIR) of the number of colorectal cancers expected using sex-specific, age-specific and calendar year-specific population incidence rates for England (Office for National Statistics, 2019a) compared to the number observed during the 10 years of follow-up, overall and by 10-year age group.

The inventors conducted analyses of the performance of the 10-year risk predictions in the 30% testing dataset for women and men separately for the: average risk model, family history model, current family history and PRS model, new family history and PRS model, and new multivariable model. The inventors also analysed the performance of the two best-performing models separately for colon cancers and rectal cancers. The inventors used Cox regression with age as the time axis to estimate the hazard ratio (HR) per SD of the log odds of the 10-year risks. The inventors used Harrell's C-index to assess the ability of the risk predictions to distinguish between affected and unaffected participants (i.e. the discrimination of the risk scores). The inventors then plotted Nelson-Aalen cumulative hazard curves for the risk scores stratified by quintile of 10-year risk.

The inventors evaluated calibration using logistic regression to estimate coefficients for the log odds of the predicted 10-year risk for the risk scores and tested whether the coefficients were equal to 1 (Van Claster et al., 2019; Huang et al., 2020). The estimated coefficient is a measure of dispersion, where values <1 indicate overdispersion, values >1 indicate under-dispersion and values close to 1 indicate no problem with dispersion. The inventors then constrained the logistic regression models to have a slope of 1 and used the intercept term to assess overall calibration (Huang et al., 2020). To illustrate the calibration of the models, the inventors drew calibration plots for deciles of the 10-year risks using the pmcalplot module (Ensor et al., 2022) in Stata.

### Utility

The inventors then conducted further analyses of the utility of the risk prediction scores. To illustrate the ability of the models to stratify colorectal cancer risk, the inventors calculated the SIR of the number of cases expected using sex- and age-specific population incidence rates for England (Office for National Statistics, 2019a) and the number observed during the 10 years of follow-up for the first four quintiles and the top two deciles of 10-year risk and using cut-offs at 1% and 2%.

The inventors conducted a decision curve analysis (Vickers and Elkin, 2006) of the survival time from baseline assessment date to either colorectal cancer diagnosis or the completion of 10 years of follow-up for the family history model, the new family history and PRS model and the new multivariable model, separately for women and men. Interpretation of the decision curves is straightforward: the curve with the higher net benefit at the threshold of interest is the better-performing risk-prediction tool. There is no need for formal statistical tests or examination of confidence intervals (Vickers et al., 2019).

### Ethics Approval

The UK Biobank has Research Tissue Bank approval (REC #11/NW/0382) that covers analysis of data by approved researchers. All participants provided written informed consent to the UK Biobank before data collection began. This research has been conducted using the UK Biobank resource under Application Number 47401.

### Example 4—Results—Models 2 and 3

After exclusions, there were 396,072 participants (214,183 women and 181,889 men) in the study dataset, 4,511 (1,913 women and 2,598 men) of whom were diagnosed with incident colorectal cancer during the follow-up period. Mean age at baseline assessment date was 56.7 years (SD=7.8 years) for unaffected women, 60.7 years (SD=6.7) for affected women, 57.3 years (SD=8.0 years) for unaffected men and 61.7 (SD=6.2 years) for affected men. For affected participants, the mean age at diagnosis was 66.4 years (SD=7.3 years) for women and 67.3 years (SD=6.7 years) for men. Affected participants had a mean follow-up time until their diagnosis of 5.7 years (SD=3.0 years) for women and 5.6 years (SD=3.0 years) for men. Unaffected participants had a mean follow-up time of 10.4 years (SD=1.2 years) for women and 10.2 years (SD=1.5 years) for men. Summary statistics for unaffected and affected women and men for the baseline risk factors considered in the development of the colorectal cancer risk prediction models are presented in Table 7.

### Training

The unadjusted HRs obtained using the 70% training dataset for the variables considered for inclusion in the models are shown in Table 8 and the new multivariable models for women and men are presented in Table 9. For both women and men, using forwards selection and backwards selection gave the same group of variables. PRS, first-degree family history of colorectal cancer, smoke ever and colorectal cancer screening were selected for the models for both women and men. Triglycerides was selected for the model for women and BMI was selected for the model for men. All selected variables were statistically significant based on Wald tests, and all were retained in the final models. The new PRS and first-degree family history models for both women and men are also shown in Table 9. For both women and men, the HRs for PRS and first-degree family history were similar in the two models. The number of imputations required to ensure that the standard errors are replicable were six for the model for women and two for the model for men (the upper limits of the 95% confidence interval for the fraction of missing information were 0.15 and 0.04, respectively).

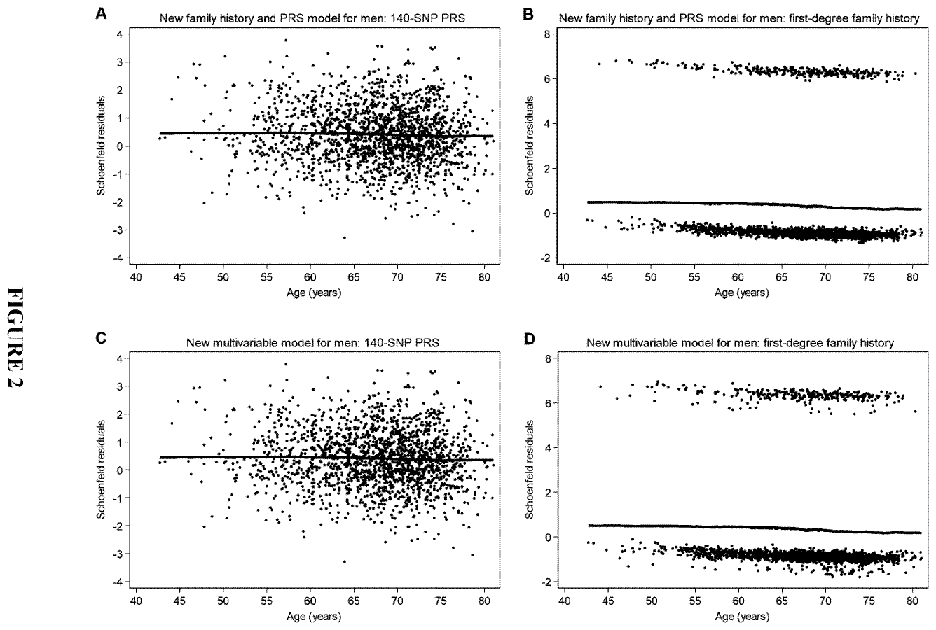

FIG. 2 shows the graphs of the Nelson-Aalen cumulative hazard function and the Cox-Snell residuals for the first imputation dataset for each of the new models. In each case, the models were good fits to the data.

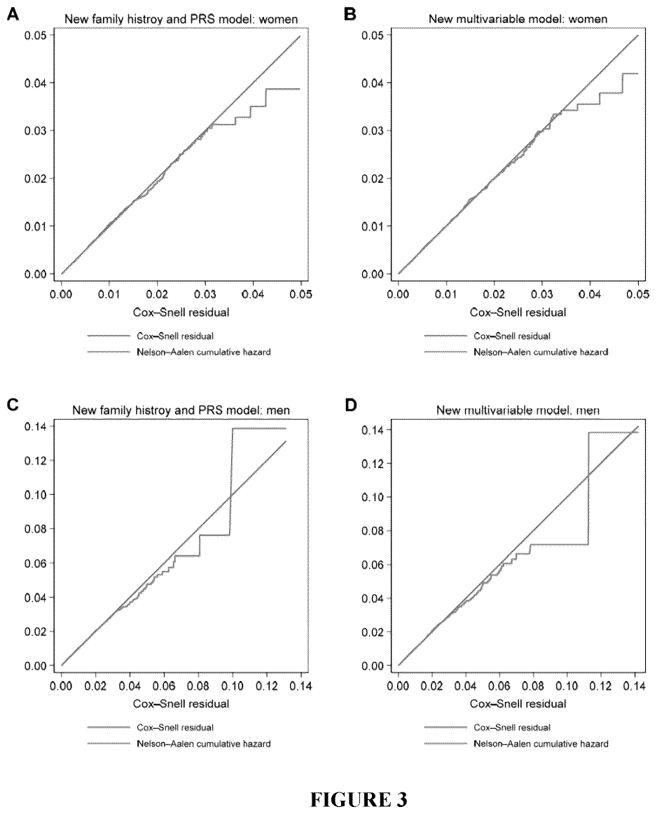

For women, fitting the risk factors as time-varying covariates did not identify any problems with the proportional hazards assumption in both of the new models. For men, first-degree family history and the 140-SNP PRS were problematic in both models (P=0.09 for family history and P=0.05 for the PRS in the new multivariable model and both P<0.001 for the new family history and PRS model. Plots of the Schoenfeld residuals in FIG. 3 showed no strong trend with age for any of the potentially problematic variables and we chose to proceed without age interactions.

TABLE 8
Unadjusted hazard ratios for women and men for the baseline risk factors
considered in the development of the colorectal cancer risk prediction
models using the multiple imputation data for the 70% training dataset.

| | Women | | | Men | | |
|---|---|---|---|---|---|---|
| | Hazard | 95% confidence | P | Hazard | 95% confidence | P |
| Risk factor | ratio | interval | value | ratio | interval | value |

Continuous

| | | | | | | |
|---|---|---|---|---|---|---|
| 140-SNP PRS (standardised) | 1.519 | 1.438, 1.605 | <0.001 | 1.500 | 1.431, 1.572 | <0.001 |
| Body mass index (natural | 1.067 | 0.786, 1.449 | 0.7 | 2.648 | 1.937, 3.620 | <0.001 |
| log of kg/m² centred) | | | | | | |
| Time since last screening | 1.019 | 0.945, 1.100 | 0.6 | 1.090 | 1.016, 1.169 | 0.02 |
| procedure, if screened in | | | | | | |
| last 10 years (years) | | | | | | |
| Physical activity (natural | 0.978 | 0.940, 1.018 | 0.3 | 0.978 | 0.947, 1.010 | 0.2 |
| log of MET-minutes per | | | | | | |
| week, centred) | | | | | | |
| Cholesterol | 1.056 | 1.007, 1.107 | 0.03 | 0.977 | 0.938, 1.019 | 0.3 |
| (mmol/L, centred) | | | | | | |
| High-density lipoprotein | 0.945 | 0.815, 1.095 | 0.5 | 0.906 | 0.779, 1.054 | 0.2 |
| (mmol/L, centred) | | | | | | |
| Low-density lipoprotein | 1.069 | 1.005, 1.137 | 0.03 | 0.963 | 0.912, 1.017 | 0.2 |
| (mmol/L, centred) | | | | | | |
| Triglycerides | 1.096 | 1.032, 1.164 | 0.003 | 1.058 | 1.016, 1.101 | 0.006 |
| (mmol/L, centred) | | | | | | |
| Cooked vegetables | 1.003 | 0.966, 1.040 | 0.9 | 1.007 | 0.979, 1.036 | 0.6 |
| (serves per day) | | | | | | |
| Salad or raw vegetables | 1.010 | 0.980, 1.040 | 0.5 | 0.979 | 0.953, 1.007 | 0.1 |
| (serves per day) | | | | | | |
| Fresh fruit | 0.992 | 0.956, 1.030 | 0.7 | 0.993 | 0.962, 1.024 | 0.6 |
| (pieces per day) | | | | | | |

Categorical

| | | | | | | |
|---|---|---|---|---|---|---|
| Affected first-degree | | | | | | |
| relative, any | | | | | | |
| No | — | | | — | | |
| Yes | 1.286 | 1.102, 1.499 | 0.001 | 1.439 | 1.271, 1.630 | <0.001 |
| Screening procedure in | | | | | | |
| last 10 years | | | | | | |
| No | — | | | — | | |
| Yes | 0.617 | 0.490, 0.778 | <0.001 | 0.683 | 0.552, 0.845 | <0.001 |
| Diabetes, type 2 | | | | | | |
| or unspecified | | | | | | |
| No | — | | | — | | |
| Yes | 1.070 | 0.811, 1.412 | 0.6 | 1.323 | 1.132, 1.547 | <0.001 |
| NSAID, regular use | | | | | | |
| No | — | | | — | | |
| Yes | 0.984 | 0.874, 1.108 | 0.8 | 0.994 | 0.901, 1.096 | 0.9 |
| Menopause and HRT | | | | | | |
| (women only) | | | | | | |
| Premenopausal | — | | | | | |
| Menopausal, no HRT | 1.306 | 1.006, 1.696 | 0.05 | | | |
| Menopausal, took HRT | 1.226 | 0.941, 1.598 | 0.1 | | | |
| Calcium supplement | | | | | | |
| No | — | | | — | | |
| Yes | 1.017 | 0.905, 1.142 | 0.8 | 0.952 | 0.846, 1.071 | 0.4 |
| Vitamin D supplement | | | | | | |
| No | — | | | | | |
| Yes | 1.043 | 0.926, 1.174 | 0.5 | 0.929 | 0.825, 1.045 | 0.2 |
| Fish oil supplement or | | | | | | |
| eat oily fish two or | | | | | | |
| more times per week | | | | | | |
| No | — | | | — | | |
| Yes | 0.991 | 0.888, 1.105 | 0.9 | 0.944 | 0.860, 1.036 | 0.2 |
| Alcohol use | | | | | | |
| Never or rarely | — | | | | | |
| One or two times | 0.958 | 0.832, 1.102 | 0.5 | 1.091 | 0.943, 1.261 | 0.2 |
| per week | | | | | | |
| Three of four times | 0.900 | 0.773, 1.047 | 0.2 | 1.146 | 0.995, 1.321 | 0.06 |
| per week | | | | | | |
| Daily or almost daily | 1.112 | 0.958, 1.291 | 0.2 | 1.300 | 1.133, 1.492 | <0.001 |
| Smoking, ever | | | | | | |
| No | — | | | | | |
| Yes | 1.240 | 1.114, 1.382 | <0.001 | 1.388 | 1.261, 1.527 | <0.001 |
| Processed meat | | | | | | |
| (serves per week) | | | | | | |
| None | — | | | — | | |
| 1 | 1.055 | 0.874, 1.273 | 0.6 | 1.200 | 0.913, 1.577 | 0.2 |
| 2 | 1.135 | 0.936, 1.376 | 0.2 | 1.324 | 1.015, 1.728 | 0.04 |
| 3 or more | 1.133 | 0.925, 1.387 | 0.2 | 1.420 | 1.092, 1.845 | 0.009 |
| Beef (serves per week) | | | | | | |
| None | — | | | — | | |
| 1 | 1.101 | 0.917, 1.322 | 0.3 | 1.219 | 0.981, 1.514 | 0.07 |
| 2 | 1.103 | 0.910, 1.335 | 0.3 | 1.180 | 0.947, 1.470 | 0.1 |
| 3 or more | 1.054 | 0.836, 1.329 | 0.7 | 1.540 | 1.218, 1.948 | <0.001 |
| Pork (serves per week) | | | | | | |
| None | — | | | — | | |
| 1 | 1.045 | 0.900, 1.213 | 0.6 | 1.019 | 0.871, 1.191 | 0.8 |
| 2 | 1.082 | 0.910, 1.287 | 0.9 | 1.186 | 1.003, 1.402 | 0.05 |
| 3 or more | 1.121 | 0.785, 1.601 | 0.6 | 1.432 | 1.118, 1.833 | 0.004 |
| Dried fruit | | | | | | |
| (serves per day) | | | | | | |
| None | — | | | — | | |
| 1 or more | 0.974 | 0.873, 1.085 | 0.6 | 0.849 | 0.767, 0.940 | 0.002 |
| Cereal (bowls per week) | | | | | | |
| None | — | | | — | | |
| 1-3 | 0.891 | 0.736, 1.079 | 0.2 | 0.901 | 0.776, 1.045 | 0.2 |
| 4-6 | 0.917 | 0.775, 1.086 | 0.3 | 0.740 | 0.643, 0.851 | <0.001 |
| 7 or more | 0.884 | 0.756, 1.033 | 0.1 | 0.721 | 0.635, 0.819 | <0.001 |
| White bread | | | | | | |
| (slices per week) | | | | | | |
| None | — | | | — | | |
| 1-4 | 1.007 | 0.726, 1.398 | 1.0 | 0.984 | 0.681, 1.420 | 0.9 |
| 5-10 | 1.005 | 0.816, 1.238 | 1.0 | 1.103 | 0.940, 1.295 | 0.2 |
| 11 or more | 1.166 | 0.969, 1.403 | 0.1 | 1.180 | 1.054, 1.320 | 0.004 |
| Wholemeal or wholegrain | | | | | | |
| bread (slices per week) | | | | | | |
| None | — | | | — | | |
| 1-4 | 0.839 | 0.695, 1.014 | 0.07 | 0.953 | 0.730, 1.243 | 0.7 |
| 5-10 | 0.917 | 0.801, 1.050 | 0.2 | 0.990 | 0.868, 1.129 | 0.9 |
| 11 or more | 0.863 | 0.751, 0.992 | 0.04 | 0.901 | 0.810, 1.002 | 0.06 |

Note:
HRT, hormone replacement therapy; MET, metabolic equivalent task; NSAID, non-steroidal anti-inflammatory drug; PRS, polygenic risk score; SNP, single-nucleotide polymorphism.

TABLE 9
Hazard ratios for the risk factors in the new models
for women and men in the 70% training dataset.

| | | | |
|---|---|---|---|
| | | 95% confidence | |
| Risk factor | Hazard ratio | interval | P value |

Women - new multivariable model

| | | | |
|---|---|---|---|
| 140-SNP PRS (standardised) | 1.515 | 1.434, 1.601 | <0.001 |
| Affected first-degree relative, any | 1.238 | 1.061, 1.444 | 0.007 |
| Smoking, ever | 1.242 | 1.115, 1.323 | <0.001 |
| Screening procedure in last 10 years, yes | 0.594 | 0.471, 0.749 | <0.001 |
| Triglycerides (mmol/L, centred) | 1.100 | 1.038, 1.166 | 0.001 |

Women - new family history and PRS model

| | | | |
|---|---|---|---|
| 140-SNP PRS (standardised) | 1.514 | 1.433, 1.599 | <0.001 |
| Affected first-degree relative, any | 1.212 | 1.039, 1.413 | 0.02 |

Men - new multivariable model

| | | | |
|---|---|---|---|
| 140-SNP PRS (standardised) | 1.492 | 1.423, 1.564 | <0.001 |
| Affected first-degree relative, any | 1.387 | 1.226, 1.570 | <0.001 |
| Smoking, ever | 1.343 | 1.220, 1.478 | <0.001 |
| Screening procedure in last 10 years, yes | 0.669 | 0.540, 0.828 | <0.001 |
| Body mass index (natural log of kg/m², | 2.410 | 1.760, 3.301 | <0.001 |
| centred) | | | |

Men - new family history and PRS model

| | | | |
|---|---|---|---|
| 140-SNP PRS (standardised) | 1.493 | 1.424, 1.565 | <0.001 |
| Affected first-degree relative, any | 1.375 | 1.214, 1.558 | <0.001 |

Note:
PRS, polygenic risk score.

Table 10 shows the HR per adjusted standard deviation for the variables in each of the models. In all models, the PRS was clearly the strongest risk factor with a HR per adjusted standard deviation of around 1.5 in each case. The other risk factors were weaker with HRs per standard deviation ranging from 1.086 to 1.177 (or the equivalent protective effect), except for first-degree family history in the family history and PRS model for women, which had an HR per adjusted standard deviation of 1.028 (P=0.3).

TABLE 10
Hazard ratios per adjusted standard deviation for the risk factors
in the new models for women and men in the 70% training dataset.

| | | | |
|---|---|---|---|
| | Hazard ratio | 95% confidence | |
| Risk factor | per adjusted SD | interval | P value |

Women - new multivariable model

| | | | |
|---|---|---|---|
| 140-SNP PRS (standardised) | 1.503 | 1.424, 1.585 | <0.001 |
| Affected first-degree relative, any | 1.086 | 1.035, 1.140 | 0.001 |
| Smoking, ever | 1.114 | 1.056, 1.174 | <0.001 |
| Screening procedure in last 10 years, yes | 0.879 | 0.824, 0.938 | <0.001 |
| Triglycerides (mmol/L, centred) | 1.082 | 1.028, 1.140 | 0.001 |

Women - new family history and PRS model

| | | | |
|---|---|---|---|
| 140-SNP PRS (standardised) | 1.497 | 1.420, 1.580 | <0.001 |
| Affected first-degree relative, any | 1.028 | 0.975, 1.083 | 0.3 |

Men - new multivariable model

| | | | |
|---|---|---|---|
| 140-SNP PRS (standardised) | 1.484 | 1.417, 1.553 | <0.001 |
| Affected first-degree relative, any | 1.126 | 1.082,1.173 | <0.001 |
| Smoking, ever | 1.177 | 1.122, 1.235 | <0.001 |
| Screening procedure in last 10 years, yes | 0.911 | 0.864, 0.961 | <0.001 |
| Body mass index (natural log of kg/m², | 1.153 | 1.101, 1.207 | <0.001 |
| centred) | | | |

Men - new family history and PRS model

| | | | |
|---|---|---|---|
| 140-SNP PRS (standardised) | 1.479 | 1.413, 1.549 | <0.001 |
| Affected first-degree relative, any | 1.120 | 1.074, 1.168 | <0.001 |

Note:
PRS, polygenic risk score; SD standard deviation.

### Testing

Summary statistics for the 10-year risk scores for women and men in the 30% testing dataset of participants with United Kingdom ancestry are shown in Table 11. The average risks (which are only based on age) had a limited range of 0.18%-1.88% for women and 0.16%-2.93% for men. Each of the risk models stratified risk more. The family history only model doubled the maximum 10-year risk for both women and men. The current family history and PRS model stratified risk the most with a maximum of 12.68% for women and 27.52% for men.

TABLE 11
Summary statistics for 10-year risk scores (%) in the 30% testing dataset.

| | | | | | |
|---|---|---|---|---|---|
| Mean | SD | Median | IQR | Minimum | Maximum |

Women

| | | | | | | |
|---|---|---|---|---|---|---|
| Average risk | 0.93 | 0.5 | 0.94 | 0.91 | 0.13 | 1.88 |
| Family history model | 0.98 | 0.67 | 0.91 | 0.85 | 0.12 | 3.89 |
| Current family history | 0.96 | 0.87 | 0.74 | 0.88 | 0.02 | 12.68 |
| and PRS model | | | | | | |
| New family history | 1.01 | 0.73 | 0.86 | 0.95 | 0.02 | 8.00 |
| and PRS model | | | | | | |
| New multivariable model | 1.04 | 0.79 | 0.85 | 0.99 | 0.02 | 10.36 |

Men

| | | | | | | |
|---|---|---|---|---|---|---|
| Average risk | 1.54 | 0.89 | 1.62 | 1.75 | 0.16 | 2.93 |
| Family history model | 1.63 | 1.17 | 1.62 | 1.64 | 0.14 | 6.05 |
| Current family history | 1.58 | 1.46 | 1.23 | 1.58 | 0.03 | 27.52 |
| and PRS model | | | | | | |
| New family history | 1.67 | 1.25 | 1.45 | 1.72 | 0.05 | 14.72 |
| and PRS model | | | | | | |
| New multivariable model | 1.72 | 1.39 | 1.43 | 1.82 | 0.04 | 14.47 |

Note:
IQR, inter-quartile range; PRS, polygenic risk score; SD, standard deviation.

Table 12 shows the performance of the models in terms of association with colorectal cancer, discrimination and calibration. The 10-year risks for all models were strongly associated with colorectal cancer, with the new multivariable model and the new family history and PRS model having a stronger association per SD than the average risk model and the other models.

TABLE 12
Performance of 10-year risk prediction scores in the 30% testing dataset.

| | Hazard ratio | 95% confidence | |
|---|---|---|---|
| Association | per SD | interval | P value |
| Women | | | |
| Average risk | 1.453 | 1.079, 1.956 | 0.01 |
| Family history model | 1.357 | 1.138, 1.617 | 0.001 |
| Current family history and PRS model | 1.864 | 1.650, 2.107 | <0.001 |
| New family history and PRS model | 2.172 | 1.871, 2.522 | <0.001 |
| New multivariable model | 2.233 | 1.935, 2.577 | <0.001 |
| Men | | | |
| Average risk | 1.112 | 0.845, 1.465 | 0.4 |
| Family history model | 1.220 | 1.023, 1.456 | 0.03 |
| Current family history and PRS model | 1.949 | 1.732, 2.193 | <0.001 |
| New family history and PRS model | 2.342 | 2.024, 2.710 | <0.001 |
| New multivariable model | 2.432 | 2.119, 2.791 | <0.001 |
| Discrimination | Harrell's | 95% confidence | P value* |
| | C-index | interval | |
| Women | | | |
| Average risk | 0.643 | 0.621, 0.664 | <0.001 |
| Family history model | 0.643 | 0.621, 0.664 | <0.001 |
| Current family history and PRS model | 0.683 | 0.663, 0.703 | <0.001 |
| New family history and PRS model | 0.683 | 0.663, 0.704 | <0.001 |
| New multivariable model | 0.690 | 0.669, 0.712 | <0.001 |
| Men | | | |
| Average risk | 0.642 | 0.624, 0.660 | <0.001 |
| Family history model | 0.641 | 0.623, 0.659 | <0.001 |
| Current family history and PRS model | 0.689 | 0.671, 0.707 | <0.001 |
| New family history and PRS model | 0.692 | 0.673, 0.710 | <0.001 |
| New multivariable model | 0.699 | 0.681, 0.717 | <0.001 |
| Calibration - slope | β | 95% confidence | P value** |
| | | interval | |
| Women | | | |
| Average risk | 0.879 | 0.718, 1.041 | 0.1 |
| Family history model | 0.735 | 0.606, 0.865 | 0.001 |
| Current family history and PRS model | 0.792 | 0.686, 0.898 | <0.001 |
| New family history and PRS model | 0.947 | 0.818, 1.075 | 0.4 |
| New multivariable model | 0.939 | 0.816, 1.062 | 0.3 |
| Men | | | |
| Average risk | 0.777 | 0.652, 0.902 | <0.001 |
| Family history model | 0.666 | 0.563, 0.770 | <0.001 |
| Current family history and PRS model | 0.766 | 0.677, 0.854 | <0.001 |
| New family history and PRS model | 0.903 | 0.796, 1.010 | 0.08 |
| New multivariable model | 0.892 | 0.791, 0.993 | 0.04 |
| Calibration - intercept | α | 95% confidence | P value |
| | | interval | |
| Women | | | |
| Average risk | −0.100 | −0.184, −0.015 | 0.02 |
| Family history model | −0.154 | −0.239, −0.070 | <0.001 |
| Current family history and PRS model | −0.132 | −0.217, −0.047 | 0.002 |
| New family history and PRS model | −0.185 | −0.270, −0.101 | <0.001 |
| New multivariable model | −0.209 | −0.294, −0.124 | <0.001 |
| Men | | | |
| Average risk | −0.151 | −0.225, 0.078 | <0.001 |
| Family history model | −0.210 | −0.284, −0.136 | <0.001 |
| Current family history and PRS model | −0.182 | −0.256, −0.108 | <0.001 |
| New family history and PRS model | −0.234 | −0.308, −0.161 | <0.001 |
| New multivariable model | −0.269 | −0.344, −0.196 | <0.001 |

Note:
*for test that Harrell's C-index = 0.5;
**for test that β = 1.

For discrimination, both of the new models performed well for women and men, as did the current family history and PRS model (Table 12). For women, the estimate of Harrell's C-index was higher for the new multivariable model compared with the new family history and PRS model (difference=0.007, P=0.02) but there was no difference between the discrimination of the current family history and PRS model and both the new multivariable model (difference=0.007, P=0.1) and the new family history and PRS model (difference=0.0004, P=0.9). For men, the new multivariable model discriminated better than the current family history and PRS model (difference=0.010, P=0.008) and the new family history and PRS model (difference=0.007, P=0.01). There was no difference in discrimination between the current family history and PRS model and the new family history and PRS model (difference=0.003, P=0.3). These three models all discriminated better than the average risk model and the family history alone model for women and men (all P<0.001).

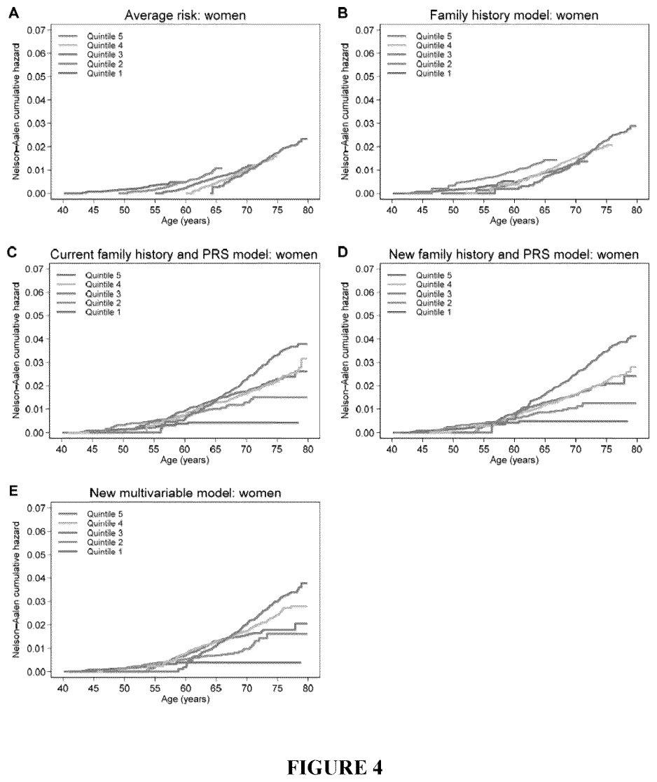

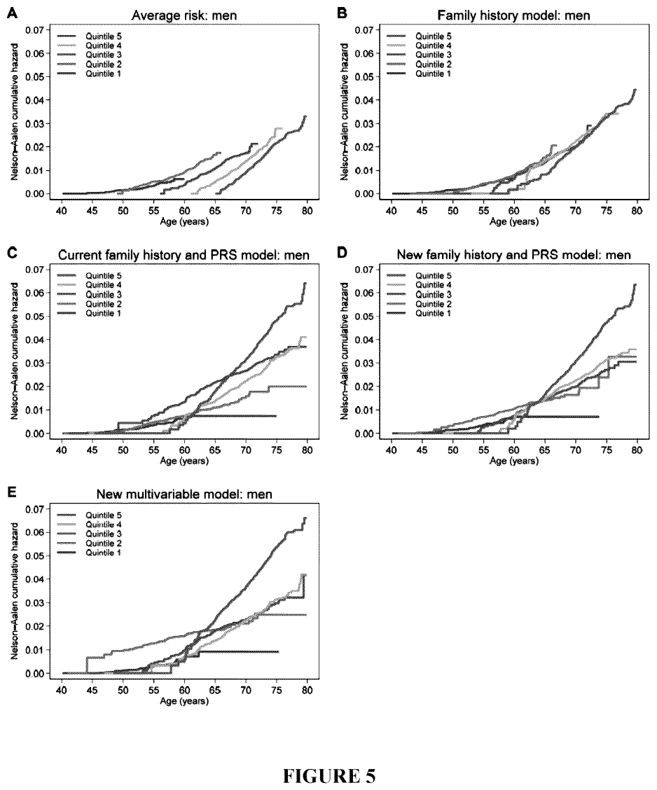

The Nelson-Aalen cumulative hazard curves for the risk scores stratified by quintile of 10-year risk for women and men are shown in FIG. 4 and FIG. 5, respectively.

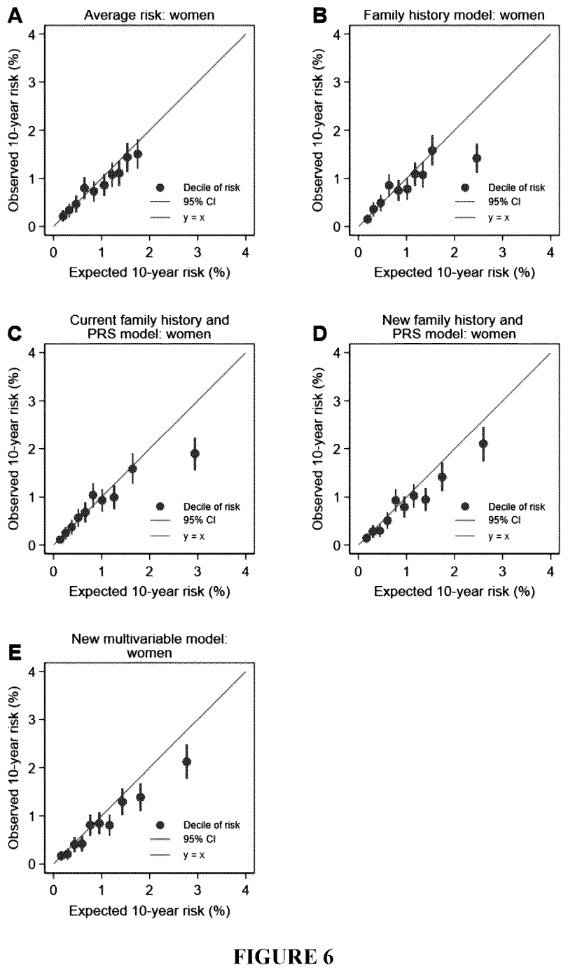

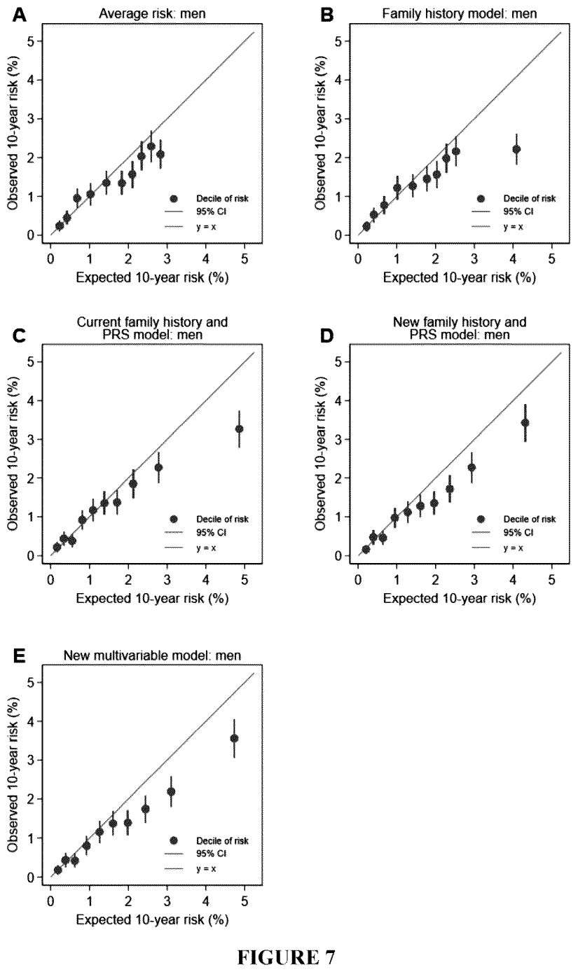

The calibration slopes for the new multivariable model and the new family history and PRS model were an improvement over the calibration slopes for the other models for both women and men (all P<0.001). For women, the calibration slopes were close to 1 for the average risk and the new models but not for the current models. For men, the slope for the new family history and PRS model was close to 1; the slope for the new multivariable model was slightly diminished (Table 12) but was a marked improvement over the other models. The intercepts were all below 0, with the intercepts for the new multivariable model being lower than the intercept for the other models for both women and men (all P<0.001). The calibration plots for women and men are shown in FIG. 6 and FIG. 7, respectively.

Analyses of the performance of the new multivariable model and the new family history and PRS model for colon cancers and rectal cancers separately showed no differences in the performance metrics (Table 13).

TABLE 13
Performance of the new multivariable model and the new family history
and PRS model for colon cancer and rectal cancer separately.

| | | | |
|---|---|---|---|
| | Hazard ratio | 95% confidence | |
| Association | per SD | interval | P value |

Colon

| | | | |
|---|---|---|---|
| Women | | | |
| New family history and PRS model | 2.204 | 1.844, 2.636 | <0.001 |
| New multivariable model | 2.271 | 1.912, 2.696 | <0.001 |
| Men | | | |
| New family history and PRS model | 2.380 | 1.973, 2.871 | <0.001 |
| New multivariable model | 2.545 | 2.133, 3.038 | <0.001 |

Rectum

| Women | | | |
|---|---|---|---|
| New family history and PRS model | 2.042 | 1.547, 2.695 | <0.001 |
| New multivariable model | 2.113 | 1.619, 2.795 | <0.001 |
| Men | | | |
| New family history and PRS model | 2.308 | 1.827, 2.915 | <0.001 |
| New multivariable model | 2.289 | 1.835, 2.854 | <0.001 |
| Discrimination | Harrell's | 95% confidence | P value* |
| | C-index | interval | |

Colon

| | | | |
|---|---|---|---|
| Women | | | |
| New family history and PRS model | 0.688 | 0.663, 0.713 | <0.001 |
| New multivariable model | 0.695 | 0.669, 0.720 | <0.001 |
| Men | | | |
| New family history and PRS model | 0.697 | 0.674, 0.721 | <0.001 |
| New multivariable model | 0.708 | 0.685, 0.731 | <0.001 |

Rectum

| Women | | | |
|---|---|---|---|
| New family history and PRS model | 0.670 | 0.633, 0.708 | <0.001 |
| New multivariable model | 0.679 | 0.640, 0.718 | <0.001 |
| Men | | | |
| New family history and PRS model | 0.681 | 0.652, 0.709 | <0.001 |
| New multivariable model | 0.683 | 0.654, 0.711 | <0.001 |
| Calibration - slope | β | 95% confidence | P value** |
| | | interval | |

Colon

| | | | |
|---|---|---|---|
| Women | | | |
| New family history and PRS model | 0.970 | 0.816, 1.124 | 0.7 |
| New multivariable model | 0.962 | 0.815, 1.110 | 0.6 |
| Men | | | |
| New family history and PRS model | 0.940 | 0.801, 1.078 | 0.4 |
| New multivariable model | 0.942 | 0.811, 1.073 | 0.4 |

Rectum

| Women | | | |
|---|---|---|---|
| New family history and PRS model | 0.880 | 0.643, 1.116 | 0.3 |
| New multivariable model | 0.878 | 0.652, 1.105 | 0.3 |
| Men | | | |
| New family history and PRS model | 0.838 | 0.670, 1.005 | 0.06 |
| New multivariable model | 0.808 | 0.651, 0.965 | 0.02 |
| Calibration - intercept | α | 95% confidence | P value |
| | | interval | |

Colon

| | | | |
|---|---|---|---|
| Women | | | |
| New family history and PRS model | −0.540 | −0.641, −0.439 | <0.001 |
| New multivariable model | −0.563 | −0.664, −0.426 | <0.001 |
| Men | | | |
| New family history and PRS model | −0.729 | −0.823, −0.635 | <0.001 |
| New multivariable model | −0.765 | −0.859, −0.671 | <0.001 |

Rectum

| | | | |
|---|---|---|---|
| Women | | | |
| New family history and PRS model | −1.439 | −1.597, −1.281 | <0.001 |
| New multivariable model | −1.463 | −1.620, −1.305 | <0.001 |
| Men | | | |
| New family history and PRS model | −1.186 | −1.304, 1.069 | <0.001 |
| New multivariable model | −1.222 | −1.340, 1.104 | <0.001 |

Note:
*for test that Harrell's C-index = 0.5;
**for test that β = 1.

### Utility

Table 14 shows that women and men in the top decile of risk are at substantially increased risk of colorectal cancer, with SIRs of 1.4-1.5 compared to population incidence rates. This result must be interpreted in context of the overall SIRs, which demonstrate the healthy volunteer effect of the UK Biobank participants. Overall, fewer 5 colorectal cancers were observed than expected using population incidence rates for both women and men (9% and 12%, respectively).

TABLE 14
Standardised incidence ratios for the new family history and
PRS model and the new multivariable model by risk group.

| | | | |
|---|---|---|---|
| | | | 95% |
| | | | confidence |
| Observed | Expected | SIR | interval |

Women

| | | | | |
|---|---|---|---|---|
| Overall (median = 0.9%) | 543 | 597.1 | 0.910 | 0.836, 0.989 |
| New family history and PRS model | | | | |
| Quintile 1 (median = 0.2%) | 27 | 42.5 | 0.635 | 0.435, 0.926 |
| Quintile 2 (median = 0.5%) | 52 | 83.5 | 0.623 | 0.475, 0.817 |
| Quintile 3 (median = 0.8%) | 111 | 126.8 | 0.875 | 0.727, 1.054 |
| Quintile 4 (median = 1.3%) | 127 | 158.2 | 0.803 | 0.675, 0.955 |
| Decile 9 (median = 1.7%) | 91 | 88.6 | 1.027 | 0.837, 1.262 |
| Decile 10 (median = 2.4%) | 135 | 97.5 | 1.385 | 1.170, 1.640 |
| New multivariable model | | | | |
| Quintile 1 (median = 0.2%) | 24 | 43.8 | 0.549 | 0.368, 0.818 |
| Quintile 2 (median = 0.5%) | 53 | 85.7 | 0.618 | 0.472, 0.809 |
| Quintile 3 (median = 0.9%) | 106 | 126.6 | 0.837 | 0.692, 1.013 |
| Quintile 4 (median = 1.3%) | 135 | 156.94 | 0.860 | 0.727, 1.018 |
| Decile 9 (median = 1.8%) | 89 | 88.1 | 1.010 | 0.821, 1.244 |
| Decile 10 (median = 2.5%) | 136 | 95.9 | 1.418 | 1.199, 1.678 |

Men

| | | | | |
|---|---|---|---|---|
| Overall (median = 1.6%) | 723 | 821.5 | 0.880 | 0.818, 0.947 |
| New family history and PRS model | | | | |
| Quintile 1 (median = 0.3%) | 35 | 43.7 | 0.801 | 0.575, 1.115 |
| Quintile 2 (median = 0.8%) | 78 | 108.1 | 0.722 | 0.578, 0.901 |
| Quintile 3 (median = 1.4%) | 131 | 184.8 | 0.709 | 0.597, 0.841 |
| Quintile 4 (median = 2.2%) | 168 | 227.2 | 0.740 | 0.636, 0.860 |
| Decile 9 (median = 2.9%) | 124 | 125.4 | 0.989 | 0.830, 1.180 |
| Decile 10 (median = 4.0%) | 187 | 132.4 | 1.413 | 1.224, 1.631 |
| New multivariable model | | | | |
| Quintile 1 (median = 0.3%) | 34 | 45.4 | 0.749 | 0.535, 1.049 |
| Quintile 2 (median = 0.8%) | 67 | 111.8 | 0.599 | 0.472, 0.762 |
| Quintile 3 (median = 1.4%) | 138 | 184.5 | 0.748 | 0.633, 0.884 |
| Quintile 4 (median = 2.2%) | 171 | 226.4 | 0.755 | 0.650, 0.877 |
| Decile 9 (median = 3.1%) | 119 | 122.9 | 0.968 | 0.809, 1.159 |
| Decile 10 (median = 4.4%) | 194 | 130.4 | 1.487 | 1.292, 1.712 |

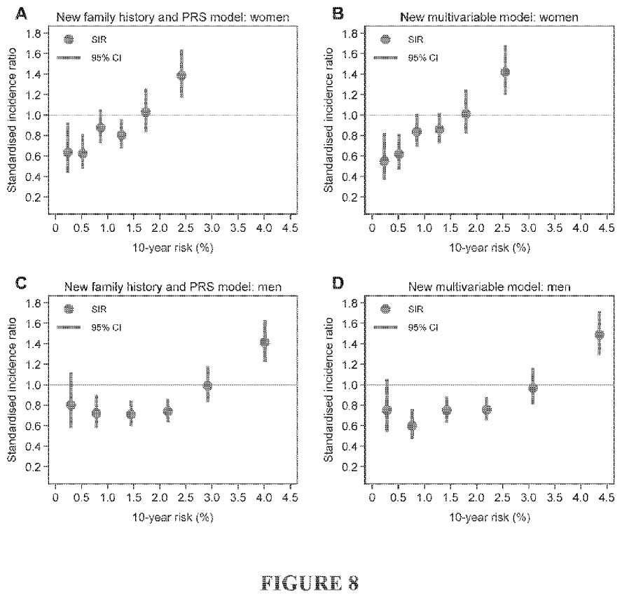

The SIRs are illustrated in FIG. 8, in which the risk groups (the first four quintiles and the top two deciles) are plotted at their median values on the x-axis. As seen in the summary statistics for the risk models, the risk predictions for men stratify risk more than the risk predictions for women. The median 10-year risk in the top decile of risk for the new multivariable model is 2.5% for women and 4.4% for men.

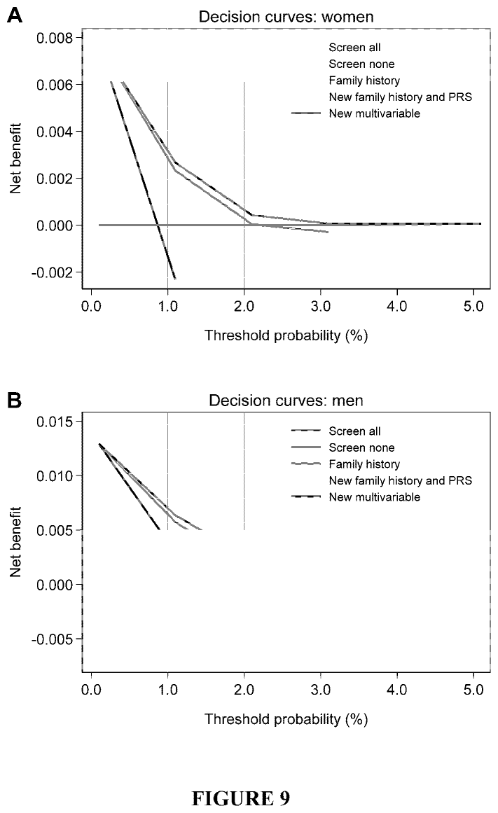

FIG. 9 shows the results of the decision curve analyses. At both the 1% and 2% risk thresholds, the new models are preferred for both women and men.

### The Model 2

The relative risk of a human female subject for developing colorectal cancer is determined using:

$$R {R}_{fhprs \_ w} = {e}^{( 0.415 \times prs ) + ( 0.192 \times deg 1 )}$$

The relative risk of a human male subject for developing colorectal cancer is determined using:

$$R {R}_{fhprs \_ m} = {e}^{( 0.4 \; 00 \times prs ) + ( 0.319 \times deg 1 )}$$

In each instance, deg1 is 1 if the subject has one or more first-degree relatives who have, or who have had, colorectal cancer, and 0 if the subject does not have one or more first-degree relatives who have, or who have had, colorectal cancer.

### The Model 3

The relative risk of a human female subject for developing colorectal cancer is determined using:

$$R {R}_{multi \_ w} = {e}^{( 0.416 \times prs ) + ( 0.219 \times deg 1 ) + ( 0.213 \times smoke ) + ( - 0.521 \times screen ) + ( 0.09 \times ( trigly - 3.296 ) )}$$

The relative risk of a human male subject for developing colorectal cancer is determined using:

$$R {R}_{multi \_ m} = {e}^{( 0.4 \times prs ) + ( 0.325 \times deg 1 ) + ( 0.3 \; 00 \times smoke ) + ( - 0.4 \; 02 \times screen ) + ( 0.873 \times ( bmi - 3.296 ) )}$$

In each instance, deg1 is 1 if the subject has one or more first-degree relatives who have, or who have had, colorectal cancer, and 0 if the subject does not have one or more first-degree relatives who have, or who have had, colorectal cancer.

In each instance, smoke is 1 if the subject has ever smoked, and 0 if the subject has not ever smoked.

In each instance, screen is 1 if the subject has had a colorectal screen in the last 10 years, and 0 if the subject has not had a colorectal screen in the last 10 years.

Trigly is the subjects blood triglyceride level in mmol/L.

Bmi is the subjects body mass index provided as the natural log of kg/m².

For each individual (aged b years), use the most recent sex-specific, country-specific and ethnicity-specific (if available) population incidence data to determine the population incidence of colorectal cancer from birth to age b years (incid_b), to age b+10 years (incid_b_10) and full lifetime from birth to age 90 years (incid_full_life). Provided below are the calculations for 10-year risks, but any other period can be chosen (e.g. 5-year risks, 15-year risks etc.).

Population incidence rates are available from, for example, the Australian Institute of Health and Welfare, the Surveillance, Epidemiology and End Results program (US), the Office for National Statistics (UK), or any other country's national cancer statistics clearinghouse.

### Cumulative Risks

$$cumul\_b = 1 - {e}^{- X \times incid \_ b}$$

$$cumul\_b \_ 10 = 1 - {e}^{- X \times incid \_ b \_ 10}$$

$$cumul\_full \_life = 1 - {e}^{- X \times incid \_ full \_ life}$$

where X is RRmulti_w, RRmulti_m, RRfhprs_w or RRfhprs_m where relevant.

### Absolute 10-Year Risk

$$crc\_risk \_ 10 yr = \frac{( cumul\_b \_ 10 - cumul\_b )}{( 1 - cumul\_b )}$$

### Absolute Remaining Lifetime Risk (to Age 90 Years)

$$crc\_risk \_rem \_life = \frac{( cumul\_full \_life - cumul\_b )}{( 1 - cumul\_b )}$$

### Absolute Full-Lifetime Risk (to Age 90 Years)

$$crc\_risk \_life = cumul\_full \_life$$

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Antoniou et al. (2003) Genet Epidemiol. 25:190-202.
Bycroft et al. (2018) Nature 562:203-209.
Coligan et al. (editors) Current Protocols in Immunology, John Wiley & Sons (including all updates until present).
Craig et al. (2003) Med Sci Sports Exerc 35:1381-1395.
Dekker et al. (2019) Lancet. 2019:394(10207):1467-80.
Devlin and Risch (1995) Genomics. 29:311-322.
Ensor et al. (2020) PMCALPLOT: Stata module to produce calibration plot of prediction model performance Available from: ideas.repec.org/c/boc/bocode/s458486.html accessed 18 Aug. 2022.
Gafni et al. (2021) PLOS One 16(9):e0251469.
Gail et al. (1989) J Natl Cancer Inst 81:1879-1886.
Glover and Hames (editors) (1995 and 1996) DNA Cloning: A Practical Approach, Volumes 1-4, IRL Press.
Hanscombe et al. (2019) PloS One 14: e02114311.
Harlow and Lane (editors) (1988) Antibodies: A Laboratory Manual, Cold Spring Harbour Laboratory.
Hopper (2015) Am J Epidemiol 182:863-867.
Huang et al. (2020) J Am Med Inform Assoc 27:621-633.
Jasperson et al. (2010) Gastroenterology 138(6):2044-58.
Keum et al. (2019) Nat Rev Gastroenterol Hepatol. 16(12):713-32.
MacInnes et al. (2013) Br J Cancer 109(5):1296-301.
MacLean et al. (2009) Nature Rev. Microbiol, 7:287-296.
Mavaddat et al. (2015) J Natl Cancer Inst 107:djv036.
Mealiffe et al. (2010) J Natl Cancer Inst 102:1618-1627.
Morozova and Marra (2008) Genomics 92:255.
Office of National Statistics. statistics (2016) Available from: www.nomisweb.co.uk/query/construct/summary.asp?mode=construct&version=0&data set=161 accessed Jan. 13, 2023.
Office for National Statistics. Cancer registration statistics (2019a) Available from: www.ons.gov.uk/peoplepopulationandcommunity/healthandsocialcare/conditionsanddiseases/datasets/cancerregistrationstatisticscancerregistrationstatisticsengland accessed Jan. 13, 2023.
Office of National Statistics. Mortality statistics-underlying cause, sex and age (2019b) Available from: www.nomisweb.co.uk/query/construct/summary.asp?mode-construct&version=0&data set=161 accessed Jan. 13, 2023.
Perbal (2000) A Practical Guide to Molecular Cloning, John Wiley and Sons.
Prive et al. (2022) Am J Hum Genet 109:12-23.
Rex et al. (2017) Am J Gastroenterol 112:1016-1030.
Roos et al. (2019) Clin Gastroenterol Hepatol. 17:2657-67 e9.
Rubin (2004) Multiple imputation for nonresponse in surveys. New York: John Wiley & Sons.
Sambrook et al. (1989) Molecular Cloning: A Laboratory Manual, Cold Spring Harbour Laboratory Press.
Schreuders et al. (2015) Gut. 64(10):1637-49.
Shaukat et al. Nat Rev Gastroenterol Hepatol. (2022) 19(8):521-31.
Slatkin and Excoffier (1996) Heredity 76:377-383.
StataCorp. Stata Statistical Software: Release 16. College Station, TX: StataCorp LLC. 2019.
Sudlow et al. (2015) PLOS Med. 2015; 12(3):e1001779.
Syngal et al. (2015) Am J Gastroenterol. 2015; 110(2):223-62; quiz 63.
Thomas et al. (2020) Am J Hum Genet. 107(3):432-44.
Tijssen (1993) Laboratory Techniques in Biochemistry and Molecular Biology—Hybridization with Nucleic Acid Probes Elsevier, New York.
Usher-Smith et al. (2015) Cancer Prev Res 9:13-26.
Van Calster et al. (2019) BMC Med 17:230.
Vickers and Elkin (2006) Med decis Making 26:565-574.
Vickers et al. (2019) Diagn Progn Res 3:18.
Voelkerding et al. (2009) Clinical Chem. 55:641-658.
Von Hippel et al. (2020) Sociol Meth Res 49:699-718.
Win et al. (2014) Gastroenterology 146:1208-1211, e1201-1205.

1. A method for assessing the risk of a human subject for developing colorectal cancer comprising:
i) performing a genetic risk assessment of the subject, wherein the genetic risk assessment involves detecting, in a biological sample derived from the subject, the presence of at least 50 single nucleotide polymorphisms, selected from Table 1 or a polymorphism in linkage disequilibrium with one or more thereof, associated with a risk of a human subject for developing colorectal cancer,
ii) performing a clinical risk assessment of the subject for developing colorectal cancer, and
iii) combining the genetic risk assessment and the clinical risk assessment to obtain the risk of a human subject for developing colorectal cancer.

2. The method of claim 1, wherein the genetic risk assessment comprises detecting the presence of at least 75, at least 100, at least 120, or each of the single nucleotide polymorphisms selected from Table 1 or a polymorphism in linkage disequilibrium with one or more thereof.

3. The method of claim 1 or claim 2, wherein the genetic risk assessment comprises detecting the presence of each of the single nucleotide polymorphisms provided in Table 1.

4. The method of any one of claims 1 to 3, wherein performing the clinical risk assessment involves obtaining information from the subject on one or more of medical history of colorectal cancer and/or polyps, age, family history of colorectal cancer and/or polyps and/or other cancer including the age of the relative at the time of diagnosis, results of previous colonoscopy and/or sigmoidoscopy, results of previous faecal occult blood test, weight, body mass index, height, sex, alcohol consumption history, smoking history, exercise history, diet, has the subject ever smoked, time since last colorectal cancer screen, blood triglyceride levels, prevalence of inflammatory bowel disease, race/ethnicity, aspirin and other NSAID use, implementation of estrogen replacement and use of oral contraceptives.

5. The method of any one of claims 1 to 3, wherein performing the clinical risk assessment involves obtaining information from the subject on first-degree relatives' history of colorectal cancer.

6. The method of any one of claims 1 to 3, wherein the subject is female and the clinical risk assessment involves obtaining information from the subject on first-degree relatives' history of colorectal cancer, has the subject ever smoked, has the subject had a colorectal cancer screen in the last 10 years and blood triglyceride levels.

7. The method of any one of claims 1 to 3, wherein the subject is male and the clinical risk assessment involves obtaining information from the subject on first-degree relatives' history of colorectal cancer, has the subject ever smoked, has the subject had a colorectal cancer screen in the last 10 years and body mass index.

6. The method of any one of claims 1 to 5, wherein the subject has had a positive fecal occult blood test.

7. The method of any one of claims 1 to 6, wherein the subject is at least 40 years old.

8. The method of any one of claims 1 to 7, wherein the subject has a family history of colorectal cancer and is at least 30 years of age.

9. The method of any one of claims 1 to 8, wherein the results of the risk assessment indicate that the subject should be enrolled in a screening program or subjected to more frequent screening.

10. The method of any one of claims 1 to 9, wherein the polymorphism in linkage disequilibrium has linkage disequilibrium above 0.9.

11. The method of any one of claims 1 to 10, wherein the polymorphism in linkage disequilibrium has linkage disequilibrium of 1.

12. The method of any one of claims 1 to 11, which further comprises comparing the risk to a pre-determined threshold.

13. The method of any one of claims 1 to 12, wherein the genetic risk assessment produces a polygenic risk score (PRS).

14. The method of claim 13, wherein the polygenic risk score is calculated as the weighted sum of the effect allele counts for the SNPs in the PRS:
$${\sum }_{j = 1}^{p} {β}_{j} {G}_{ij}$$
where βⱼ is the weight for SNP j, Gᵢⱼ is the count (0, 1, 2) of the effect alleles of SNP j for individual i, and p is the number of SNPs in the PRS, and then PRS is standardised to have a mean of 0 and standard deviation of 1 by subtracting the population mean and dividing by the population standard deviation:
$$standard P R S = \frac{P R {S}_{raw} - P R {S}_{\overline{x}}}{P R {S}_{sd}} ,$$
where PRS_{raw} is the individual's raw PRS, PRS_{x̅} is the population mean of PRS_{raw}, and PRS_{sd} is the population standard deviation of PRS_{raw}.

15. The method of claim 13, wherein the polygenic risk score is determined using an odds ratio (OR) for each effect allele and effect allele frequency (p).

16. The method of claim 15, wherein for each polymorphism the unscaled population average risk (μ) is calculated as: μ=(1−p)²+2p(1−p)OR+p²OR².

17. The method of claim 16, wherein an adjusted risk for each polymorphism is calculated as
$$\frac{O {R}^{N}}{μ} ,$$
where N is the number of effect alleles.

18. The method of claim 17, wherein the polygenic risk score is determined by combining the adjusted risk for each polymorphism.

19. The method of claim 18, wherein the adjusted risk for each polymorphism are combined by multiplication to produce prs_rr.

20. The method of any one of claims 1 to 19, wherein the clinical risk assessment based on whether the subject has or does not have at least one first degree relative who has, or has had, colorectal cancer (fh_rr).

21. The method of any one of claim 1 to 14 or 20, wherein the subject is female and the clinical and genetic relative risk assessments are combined by determining:
$$R {R}_{fhprs \_ w} = {e}^{( PCDE 1 \times prs ) + ( PDCE 2 \times deg 1 )}$$
where:
PDCE1 is a predetermined β coefficient for the genetic risk assessment for a female subject,
PDCE2 is a predetermined β coefficient for a female subject who has at least one first-degree relative who has, or has had, colorectal cancer, and
deg1 identifies whether the subject has one or more first-degree relatives who have, or who have had, colorectal cancer.

22. The method of any one of claim 1 to 14 or 20, wherein the subject is male and the clinical and genetic relative risk assessments are combined by determining:
$$R {R}_{fhprs \_ w} = {e}^{( PCDE 3 \times prs ) + ( PDCE 4 \times deg 1 )}$$
where:
PDCE3 is a predetermined β coefficient for the genetic risk assessment for a male subject,
PDCE4 is a predetermined β coefficient for a male subject has at least one first degree relative who has, or has had, colorectal cancer, and
deg1 identifies whether the subject has one or more first-degree relatives who have, or who have had, colorectal cancer.

23. The method of claim 20, wherein the genetic risk assessment and the clinical risk assessment are combined using the formula crc_rr=prs_rr×fh_rr.

24. The method of any one of claims 1 to 14, wherein the subject is female and the clinical and genetic relative risk assessments are combined by determining:
$$R {R}_{multi \_ w} = {e}^{( PDCE 5 \times prs ) + ( PDCE 6 \times deg 1 ) + ( PDCE 7 \times smoke ) + ( PDCE 8 \times screen ) + ( PDCE 9 \times ( trigly - 3.296 ) )}$$
where:
PDCE5 is a predetermined β coefficient for the genetic risk assessment for a female subject,
PDCE6 is a predetermined β coefficient for a female subject who has at least one first-degree relative who has, or has had, colorectal cancer,
PDCE7 is a predetermined β coefficient for a female subject who has been, or is, a smoker,
PDCE8 is a predetermined β coefficient for a female subject who has had a colorectal cancer screen in the past 10 years,
PDCE9 is a predetermined β coefficient for a female subject's triglyceride levels (mmol/L),
deg1 is if the female subject has one or more first-degree relatives who have, or who have had, colorectal cancer
smoke is if the female subject has ever smoked,
screen is if the female subject has had a colorectal screen in the last, for example, 10 years, and
trigly is the female subject's blood triglyceride level in mmol/L.

25. The method of any one of claims 1 to 14, wherein the subject is male and the clinical and genetic relative risk assessments are combined by determining:
$$R {R}_{multi \_ m} = {e}^{( PDCE 10 \times prs ) + ( PDCE 11 \times deg 1 ) + ( PDCE 12 \times smoke ) + ( PDCE 13 \times screen ) + ( PDCE 14 \times ( bmi - 3. 296 ) )}$$
where:
PDCE10 is a predetermined β coefficient for the genetic risk assessment for a male subject,
PDCE11 is a predetermined β coefficient for a male subject who has at least one first-degree relative who has, or has had, colorectal cancer,
PDCE12 is a predetermined β coefficient for a male subject who has been, or is, a smoker,
PDCE13 is a predetermined β coefficient for a male subject who has had a colorectal cancer screen in the past 10 years,
PDCE14 is a predetermined β coefficient for a male subject's body mass index (natural log of kg/m²),
deg1 is if the male subject has one or more first-degree relatives who have, or who have had, colorectal cancer
smoke is if the male subject has ever smoked,
screen is if the male subject has had a colorectal screen in the last, for example, 10 years, and
bmi is the subject's body mass index expressed as the natural log of kg/m².

26. The method of any one of claims 1 to 25 which comprises determining one or more or all of the absolute 5-year risk, the absolute 10-year risk, the absolute remaining lifetime risk (to age 90) or the absolute full-lifetime risk (to age 90).

27. A computer-implemented method for assessing the absolute risk of a human subject for developing colorectal cancer, the method operable in a computing system comprising a processor and a memory, the method comprising:
receiving clinical risk data and genetic risk data for the subject, wherein the clinical and genetic risk data was obtained by a method of any one of claims 1 to 21;
processing the data to combine the clinical risk data with the genetic risk data to obtain the relative risk of a human subject for developing colorectal cancer;
outputting the absolute risk of a human subject for developing colorectal cancer.

28. The computer-implemented method of claim 27, wherein the clinical risk data and genetic risk data for the subject is received from a user interface coupled to the computing system.

29. The computer-implemented method of claim 27 or claim 28, wherein the clinical risk data and genetic risk data for the subject is received from a remote device across a wireless communications network.

30. The computer-implemented method of any one of claims 27 to 29, wherein outputting comprises outputting information to a user interface coupled to the computing system.

31. The computer-implemented method of any one of claims 27 to 30 which comprises determining a genetic risk score based on genetic data derived from a biological sample taken from the subject.

32. A computer-readable storage medium storing executable code, wherein when a processor executes the code, the processor is caused to perform the method of any one of claims 27 to 31.

33. A device for assessing the risk of a human subject developing colorectal cancer, the device comprising:
a processor; and
a memory device storing executable code, the memory being accessible to the processor;
wherein, when caused to execute the executable code stored in the memory device, the processor is caused to perform a method of any one of claims 1 to 26.

34. The device of claim 33 further comprising a display component, wherein the processor is further caused to display the colorectal cancer risk score of the subject for developing colorectal cancer on the display component.

35. The device of claim 33 or claim 34 further comprising a communications module, wherein the processor is further caused to communicate the colorectal cancer risk score of the subject for developing colorectal cancer to an external device via the communications module.

36. A method for determining the need for routine diagnostic testing of a human subject for colorectal cancer comprising assessing the risk of the subject for developing colorectal cancer using the method of any one of claims 1 to 36.

37. A method of screening for colorectal cancer in a human subject, the method comprising assessing the risk of the subject for developing colorectal cancer using the method of any one of claims 1 to 36, and routinely screening for colorectal cancer in the subject if they are assessed as having a risk for developing colorectal cancer.

38. A method for determining the need of a human subject for prophylactic anti-colorectal cancer therapy comprising assessing the risk of the subject for developing colorectal cancer using the method of any one of claims 1 to 27.

39. A method for preventing colorectal cancer in a human subject, the method comprising assessing the risk of the subject for developing colorectal cancer using the method of any one of claims 1 to 27, and administering an anti-colorectal cancer therapy to the subject if they are assessed as having a risk for developing colorectal cancer.

40. An anti-colorectal cancer therapy for use in preventing colorectal cancer in a human subject at risk thereof, wherein the subject is assessed as having a risk for developing colorectal cancer using the method of any one of claims 1 to 27.

41. A method for stratifying a group of human subjects for a clinical trial of a candidate therapy, the method comprising assessing the individual risk of the subjects for developing colorectal cancer using the method of any one of claims 1 to 27, and using the results of the assessment to select subjects more likely to be responsive to the therapy.

42. A genetic array comprising at least one probe comprising a sequence of nucleotides selected from those provided as SEQ ID NOs 1 to 140.
